(19) United States
(12) Patent Application Publication
(10) Pub. No.: US 2003/0176494 A1
(43) Pub. Date: Sep. 18, 2003

(54) Novel coumarin and chromene compounds and methods of preparation and use thereof for treating or preventing viral infections

(76) Inventors: Ze-Qi Xu, Woodridge, IL (US); Hongwei Yuan, San Mateo, CA (US); Jennifer Crabb, Chicago, IL (US); Raghu Samy, Aurora, IL (US); Ailing Li, Aurora, IL (US); Hua Cao, Chicago, IL (US); Jeff Deignan, Chicago, IL (US); Lihui Sun, Audubon, PA (US)

Correspondence Address: Emily Miao, McDonnell Boehnen Hulbert & Berghoff, 32nd Floor, 300 S. Wacker Drive, Chicago, IL, 60606, US

(21) Appl. No.: 10/295,340

(22) Filed: Nov. 15, 2002

Related U.S. Application Data
(60) Provisional application No. 60/332,655, filed on Nov. 16, 2001.

(51) Int. Cl. A61K 31/366; A61K 31/655; A61K 31/55; A61K 31/451; A61K 31/4025

(52) U.S. Cl. 514/455; 514/217.03; 514/422; 514/321; 514/151

(57) ABSTRACT

The present invention relates to methods of preparation and use of coumarin and chromene compounds for treating or preventing viral infections.

### RELATED APPLICATIONS

This application claims priority to U.S. Provisional Application No. 60/332,655, filed Nov. 16, 2001.

### FIELD OF THE INVENTION

This invention relates to novel coumarin and chromene compounds, methods of their preparation, and their use in treating or preventing viral infections.

### BACKGROUND OF THE INVENTION

Viruses are important etiologic agents in infectious disease in humans and other mammals, and comprise a diverse group that range widely in size, shape, chemical composition, host range, and effects on hosts. After several decades of study, only a limited number of antiviral agents are available for the treatment and/or prevention of diseases caused by viruses such as HIV, hepatitis B, herpes simplex type 1 and 2, cytomegalovirus, varicella zoster virus, Epstein Barr virus, influenza A and B, parainfluenza, adenovirus, measles, and respiratory syncytial virus. Because of their toxic effects on a host, many antiviral agents are limited to topical applications. Accordingly, there is a need for safe and effective anti-viral agents with a wide-spectrum of anti-viral activity with reduced toxicity to the host.

A. Human Immunodeficiency Virus (HIV)

Human immunodeficiency virus (HIV), which was also called human T-lymphotropic virus type III (HTLV-III), lymphadenopathy-associated virus (LAV) or AIDS-associated retrovirus (ARV), was first isolated in 1982 and has been identified as the etiologic agent of the acquired immunodeficiency syndrome (AIDS) and related diseases. Since then, chemotherapy of AIDS has been one of the most challenging scientific endeavors. So far, fourteen drugs have been approved by FDA and are being clinically used as drugs for the treatment of AIDS and AIDS-related complex. Although these FDA-approved drugs can extend the life of AIDS patients and improve their quality of life, none of these drugs are capable of curing the disease. Side effects as well as the emergence of drug-resistant viral strains limit the long-term use of these agents.¹ On the other hand, the number of AIDS patients worldwide has increased dramatically within the past decade and estimates of the reported cases in the very near future also continue to rise dramatically. It is therefore apparent that there is a great need for other promising drugs having improved selectivity and activity to combat AIDS.¹ Several approaches including chemical synthesis, natural products screening, and biotechnology have been utilized to identify compounds targeting different stages of HIV replication for therapeutic intervention.²

B. Hepatitis B Virus (HBV)

The hepatitis B virus (HBV) infects people of all ages. It is one of the fastest-spreading sexually transmitted diseases, and also can be transmitted by sharing needles or by behavior in which a person's mucus membranes are exposed to an infected person's blood, semen, vaginal secretions, or saliva. While the initial sickness is rarely fatal, ten percent of the people who contract hepatitis are infected for life and run a high risk of developing serious, long-term liver diseases, such as cirrhosis of the liver and liver cancer, which can cause serious complications or death.⁴ The World Health Organization lists HBV as the ninth leading cause of death. It is estimated that about 300 million persons are chronically infected with HBV worldwide, with over 1 million of those in the United States. The Center for Disease Control and Prevention estimates that over 300,000 new cases of acute HBV infection occurs in the United States each year, resulting in 4,000 deaths due to cirrhosis and 1,000 due to hepatocellular carcinoma.⁵ The highest rates of HBV infections occur in Southeast Asia, South Pacific Islands, Sub-Saharan Africa, Alaska, Amazon, Bahai, Haiti, and the Dominican Republic, where approximately 20% of the population is chronically infected.⁶

Hepatitis B virus (HBV) infection is currently the most important chronic virus infection, but no safe and effective therapy is available at present. The major therapeutic option for carriers of HBV is alpha interferon, which can control active virus replication. However, even in the most successful studies, the response rate in carefully selected patient groups has rarely exceeded 40%.^{7, 8} One of the reasons cited for interferon failure is the persistence of viral supercoiled DNA in the liver.⁹ Clinical exploration of many promising antiviral agents such as nucleoside analogues is hampered because their aspecific body distribution leads to significant toxic side effects. Recently, a new nucleoside analogue, 2′,3′-dideoxy-3′-thiacytidine (3TC), was approved to treat HBV infection with only minimal side effects.¹⁰⁻¹²

C. Influenza Virus

Influenza is a viral infection marked by fever, chills, and a generalized feeling of weakness and pain in the muscle, together with varying signs of soreness in the respiratory tract, head, and abdomen. Influenza is caused by several types of myxoviruses, categorized as groups A, B, and C₄. These influenza viruses generally lead to similar symptoms but are completely unrelated antigenically, so that infection with one type confers no immunity against the other. Influenza tends to occur in wavelike epidemics throughout the world; influenza A tends to appear in cycles of two to three years and influenza B in cycles of four to five years. Influenza is one of the few common infectious diseases that are poorly controlled by modem medicine. Its annual epidemics are occasionally punctuated by devastating pandemics. For example, the influenza pandemic of 1918, which killed over 20 million people and affected perhaps 100 times that number, was the most lethal plague ever recorded. Since that time, there have been two other pandemics of lesser severity, the so-called Asian flu of 1957 and the Hong Kong flu of 1968. All of these pandemics were characterized by the appearance of a new strain of influenza virus to which the human population had little resistance and against which previously existing influenza virus vaccines were ineffective. Moreover, between pandemics, influenza virus undergoes a gradual antigenic variation that degrades the level of immunological resistance against renewed infection.¹³

Anti-influenza vaccines, containing killed strains of types A and B virus currently in circulation, are available, but have only a 60 to 70% success rate in preventing infection. The standard influenza vaccine has to be redesigned each year to counter new variants of the virus. In addition, any immunity provided is short-lived. The only drugs currently effective in the prevention and treatment of influenza are amantadine hydrochloride and rimantadine hydrochloride.¹⁴⁻¹⁶ While the clinical use of amantadine has been limited by the excess rate of CNS side effects, rimantadine is more active against influenza A both in animals and human beings, with fewer side effects.^{17, 18} It is the drug of choice for the chemoprophylaxis of influenza A.^{13, 19, 20} However, the clinical usefulness of both drugs is limited by their effectiveness against only influenza A viruses, by the uncertain therapeutic efficacy in severe influenza, and by the recent findings of recovery of drug-resistant strains in some treated patients.²¹⁻²⁵ Ribavirin has been reported to be therapeutically active, but it remains in the investigational stage of development.^{26, 27}

D. Cytomegalovirus (CMV)

Cytomegalovirus (CMV) is a member of the herpes virus family, other well-known members of which include herpes simplex virus, types I and II, Epstein Barr virus, and Varicella Zoster virus. Although these viruses are related taxonomically, all comprising double-stranded DNA viruses, infections due to these viruses manifest in clinically distinct ways. In the case of CMV, medical conditions arising from congenital infection include jaundice, respiratory distress and convulsive seizures that may result in mental retardation, neurologic disability or death. Infection in adults is frequently asymptomatic, but may manifest as mononucleosis, hepatitis, pneumonitis or retinitis, particularly in immunocompromised patients such as AIDS sufferers, chemotherapy patients and organ transplant patients undergoing tissue rejection therapy.

Up to 45% of all HIV-infected persons will develop cytomegalovirus-induced disease before their lives end.²⁸ Although two antiviral agents—ganciclovir and foscarnet are available to treat human cytomegalovirus (HCMV), they act as virustatic agents to slow but not halt progression of disease; hence, disease routinely progresses despite daily maintenance with either agent. Moreover, therapy using either agent is problematic because both agents are associated with serious toxicities.²⁹

Drug therapies have generally focused upon interactions with proteins in efforts to modulate their disease-causing or disease-potentiating functions. Such therapeutic approaches have failed for cytomegalovirus infections. Effective therapy for CMV has not yet been developed despite studies on a number of antiviral agents. Interferon, transfer factor, adenine arabinoside (Ara-A), acycloguanosine (Acyclovir) and certain combinations of these drugs have been ineffective in controlling CMV infections. Based on preclinical and clinical data, foscarnet and ganciclovir show limited potential as antiviral agents. Foscarnet treatment has resulted in the resolution of CMV retinitis in five AIDS patients to date. Ganciclovir studies have shown efficacy against CMV retinitis and colitis. However, though ganciclovir seems to be well tolerated by most treated individuals, the appearance of a reversible neutropenia, the emergence of resistant strains of CMV upon long-term administration, and the lack of efficacy against CMV pneumonitis limit the long term applications of this compound. Cidofovir was approved to treat HCMV in certain AIDS patients due to its undesired toxicities. The development of more effective and less toxic therapeutic compounds and methods is needed for both acute and chronic use.

Several HCMV vaccines have been developed or are in the process of development. Vaccines based on live attenuated strains of HCMV have been described. A proposed HCMV vaccine using a recombinant vaccinia virus expressing HCMV glycoprotein B has also been described. However, vaccinia models for vaccine delivery are believed to cause local reactions. Additionally, vaccinia vaccines are considered possible causes of encephalitis.

E. Other Herpes Viruses

Varicella zoster virus (VZV) is the etiologic agent that produces both varicella (chickenpox) and zoster (shingles). As with other herpes viruses, VZV causes both an acute illness and lifelong latent infection. Acute primary infection (varicella) typically occurs during childhood, where the resulting infection is relatively mild. Conversely, primary infection in adults can be more severe. Herpes zoster cutaneous eruptions are caused by reactivation of VZV present in sensory ganglia.³⁰ Herpes zoster occurs more frequently with elderly and immunosuppressed individuals, and is eight times more likely to develop in HIV-infected individuals than in other individuals in comparable age groups.³¹

Along with other immunosuppressed patients, HIV-infected patients may develop severe and in certain cases life-threatening illnesses following either primary or recurrent VZV infection. Therapy for HIV-infected patients experiencing VZV infection generally involves administering acyclovir or vidarabine (Ara-A), with hospitalization required in many instances. To inhibit VZV replication, serum levels of acyclovir are about ten times greater than those needed to inhibit Herpes Simplex Type 1 and 2.

Herpes simplex virus type 1 and type 2 (HSV-1 and HSV-2) can establish latency following primary infection and can thus subsequently reactivate to induce recurrent disease. Upon primary infection, herpes simplex type I induces diseases including primary gingivostomatitis, encephalitis, and kerato-conjunctivitis, while herpes simplex type 2 induces primary genital herpes and neonatal herpes. Upon recurrence, herpes simplex type 1 induces diseases including recurrent oral herpes and recurrent kerato-conjunctivitis, while herpes simplex type 2 induces recurrent genital herpes.³² HSV infection in HIV-infected patients can produce widespread and occasionally life-threatening lesions.

Acyclovir, delivered either intravenously, orally, or topically, shortens clinical illness in both immunocompetent and immunosuppressed patients. Vidarabine also has been used in treating HSV. Some vaccine strategies have been investigated with a view towards preventing initial primary infection. However, protecting only against primary disease but not protecting against latency and subsequent recurrence is inadequate for those persons already initially infected. Moreover, acyclovir-resistant HSV infections recently have been observed, in many cases occurring among HIV-infected patients treated successfully with acyclovir in the past. The existence of such acyclovir-resistant infections in HIV-infected patients is troubling in view of the limited number of alternative therapeutic options available.

Respiratory Syncytial Virus (RSV) is the prime etiologic agent producing lower respiratory tract disease. RSV causes extensive yearly epidemics during which there is a marked increase in hospital admissions of patients, especially infants and young children, experiencing severe lower respiratory tract disease. Immunosuppressed patients infected with RSV are at high risk of mortality. Ribavirin is the only currently approved drug for treating RSV infections. However, this drug appears to have limited efficacy. Additionally, development of effective vaccines has proven difficult to date.

F. Opportunistic Infections

The viruses described above can act as sole causes of infection or can act to produce opportunistic infections in patients already battling immunosuppressing infections such as HIV. Acting by themselves, these viruses can present therapeutic challenges. But when acting to produce opportunistic infections in HIV-infected or other immunosuppressed patients, these viruses dramatically increase the difficulty and complexity of successful treatment.

In addition to the viruses discussed above, other viral, bacterial, fungal, and protozoal pathogens can induce opportunistic infections. Common opportunistic pathogens in addition to those described above include Mycobacterium avium complex (MAC), Pneumocystis carinii (PC), and M. tuberculosis.

Present therapies for HIV-infected patients also suffering from opportunistic infection generally involve administering a plurality of antiviral compounds. In such a treatment regimen, termed combination therapy, each antiviral compound employed demonstrates best antiviral activity against a distinct viral infection. For example, a combination therapy of AZT and ganciclovir can be used for an HIV-infected patient also experiencing CMV retinitis, where AZT targets the HIV infection and ganciclovir targets the CMV infection. Thus, combination therapies can be powerful therapeutic tools. Even more powerful and desirable, however, would be a single antiviral compound that demonstrates antiviral activity against both HIV and other viruses.

While some limited success has been realized in the search for viable therapeutics for treatment of the viral infections discussed above, therapeutic agents for many viruses remain severely limited. Furthermore, there are no known safe and therapeutic treatments for HBV, influenza and HIV. In HBV, with the possible exception of the drug 3TC, the use of nucleoside-based antiviral agents leads to toxicity, probably due to cross-inhibition of cellular mitchondrial DNA. Clearly, there is a need for a new class of antiviral agents which could minimize the toxicity associated with cross-inhibition. In influenza, amantadine and rimantadine have been shown to be moderately effective against only influenza A viruses, with amantadine having excessive side effects. Recently, strains of influenza A resistant to amantadine and rimantadine have been isolated. Accordingly, there is a need for new types of therapeutic antiviral agents particularly against both influenza A and influenza B, as well as against HIV, HBV and HIV and other viruses. Furthermore, due to the loss of CD4 T lymphocytes in an HIV infected person, leading to immunodeficiency and thus increasing susceptibility to a broad range of opportunistic viral, bacterial, fungal, and protozoal pathogens, identifying anti-HIV agents having a spectrum of antiviral and antimicrobial activities is of particular interest. These agents would be not only effective against HIV infection, but also effective against or preventive of opportunistic infections in AIDS patients.

A class of coumarin compounds, either natural products isolated from several tropical plants of the genus Calophyllum^{3, 33-38} or synthetic analogues,³⁹⁻⁴¹ have been demonstrated to be active against HIV-1 and other viruses.⁴² (+)-Calanolide A (1), isolated from the from the rain forest tree Calophyllum lanigerum, is the most active one in this class against HIV-1.³ For example, (+)-calanolide A demonstrated 100% protection against the cytopathic effects of HIV-1, one of two distinct types of HIV, down to a concentration of 0.1 μM. This agent also halted HIV-1 replication in human T-lymphoblastic cells (CEM-SS)(EC₅₀=0.1 μM/IC₅₀=20 μM).³ More interestingly and importantly, (+)-calanolide A was found to be active against both the AZT-resistant G-9106 strain of HIV as well as the pyridinone-resistant A17 virus.³ Thus, the calanolides, classified as HIV-1 specific reverse transcriptase inhibitors, represent novel anti-HIV chemotherapeutic agents for drug development and (+)-calanolide A has been selected for further pharmacological and clinical development.^{43,44} However, a natural source of (+)-calanolide A is limited.³⁵ This limited availability fueled the desire to develop practical synthesis routes to enable further study and development to be carried out on this active and promising series of compounds.

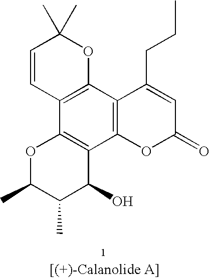

Herein we describe new coumarin and chromene compounds and methods for their use for treating or preventing viral infections.

### SUMMARY OF THE INVENTION

The present invention relates to novel anti-viral coumarin and chromene compounds and methods of use in treating antiviral infections. These new coumarin and chromene compounds are useful in preparing calanolide derivatives as described in WO 00/64902, WO 00/64903, and U.S. Pat. No. 6,369,241, which are incorporated herein by reference.

Accordingly, one object of the invention is to provide a method for treating or preventing a viral infection comprising administering to a subject in need of such therapy an anti-viral effective amount of a compound of formula I wherein the compounds of formula I comprise:

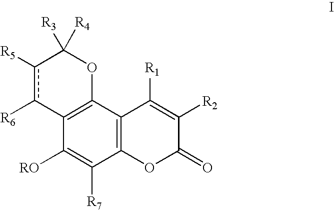

wherein R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl) amino-C₁₋₈ alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen;

R₂ is H, halogen, hydroxyl, amino, thio, cyano, C₁₋₆ alkyl, aryl-C₁₋₆ alkyl, mono- or poly-fluorinated C₁₋₆ alkyl, aryl or heterocycle; or

R₁ and R₂ together can form a 5-7 membered saturated or unsaturated cyclic ring or heterocyclic ringcyclic ring;

R₃ and R₄ are independently selected from the group consisting of H, C₁₋₆ alkyl, aryl-C₁₋₆ alkyl, mono- or poly-fluorinated C₁₋₆ alkyl, aryl or heterocycle; or R₃ and R₄ together form a 5-7 membered saturated or unsaturated cyclic ring or heterocyclic ringcyclic ring;

R₅ and R₆ are independently selected from the groups consisting of H, halogen, hydroxyl, amino, nitro, azido, C₁₋₆ alkyl, aryl-C₁₋₆ alkyl, mono- or poly-fluorinated C₁₋₆ alkyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen;

R₇ is H, halogen, hydroxyl, amino, nitro, thio, cyano, azido, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, —C(O)R₈, —SO₂R₈, —P(O)(OR₈)₂, —P(O)(OR)(OR₉), —R₈C(O)R₉, —R₈SO₂R₉, or —R₈P(O)(OR₉)₂.

R is H, C₁₋₆ alkyl, aryl-C₁₋₆ alkyl, mono- or poly-fluorinated C₁₋₆ alkyl, aryl, heterocycle, —C(O)R₈, —SO₂R₈, —P(O)(OR₈)₂, —P(O)(OR₈)(OR₉), —R₈C(O)R₉ or —R₈SO₂R₉, —R₈P(O)(OR₉)₂, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen; and

R₈ and R₉ are independently selected from the groups consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl) amino-C₁₋₈ alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen.

Another object of the invention is to provide a method for treating or preventing a viral infection comprising administering to a subject in need of such therapy an anti-viral effective amount of a compound of formula II wherein the compounds of formula II comprise:

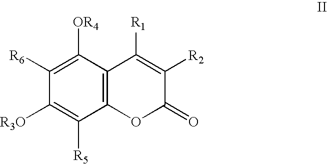

wherein

R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl) amino-C₁₋₈ alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C-8 alkylamino-C₁₋₈ alkyl, di(C₁₋₆ alkyl)amino-C₁₋₈ alkyl, nitro, thio, cyano, azido or halogen;

R₂ is H, halogen, hydroxyl, amino, thio, cyano, C₁₋₆ alkyl, aryl-C₁₋₆ alkyl, mono- or poly-fluorinated C₁₋₆ alkyl, aryl or heterocycle; or

R₁ and R₂ together can form a 5-7 membered saturated or unsaturated cyclic ring or heterocyclic ring;

R₃ and R₄ are independently selected from the group consisting of H, C₁₋₆ alkyl, aryl-C₁₋₆ alkyl, mono- or poly-fluorinated C₁₋₆ alkyl, aryl, heterocycle, —C(O)R₇, —SO₂R₇, —P(O)(OR₇)₂, —P(O)(OR₇)(OR₈), —R₇C(O)R₈, —R₇SO₂R₈, —R₇P(O)(OR₈)₂, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen;

R₅ and R₆ are independently selected from the group consisting of H, halogen, hydroxyl, amino, nitro, azido, C₁₋₆ alkyl, aryl-C₁₋₆ alkyl, mono- or poly-fluorinated C₁₋₆ alkyl, aryl, heterocycle, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, —C(O)R₇, —SO₂R₇, —P(O)(OR₇)₂, —P(O)(OR₇)(OR₈), —R₇C(O)R₈, —R₇SO₂R₈, or —R₇P(O)(OR₈)₂.

R₇ and R₈ are independently selected from the group consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen.

Another object of the invention is to provide a method for treating or preventing a viral infection comprising administering to a subject in need of such therapy an anti-viral effective amount of a compound of formula III wherein the compounds of formula III comprise:

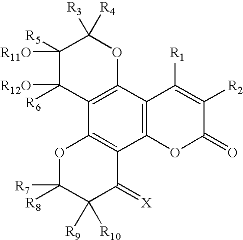

wherein

R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C, s alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen;

R₂ is H, halogen, hydroxyl, amino, thio, cyano, C₁₋₆ alkyl, aryl-C₁₋₆ alkyl, mono- or poly-fluorinated C₁₋₆ alkyl, aryl or heterocycle; or

R₁ and R₂ together can form a 5-7 membered saturated or unsaturated cyclic ring or heterocyclic ring;

R₅ and R₆ are independently selected from the group consisting of H, C₁₋₆ alkyl, aryl-C₁₋₆ alkyl, mono- or poly-fluorinated C₁₋₆ alkyl, aryl or heterocycle; or R₅ and R₆ together form a 5-7 membered saturated or unsaturated cyclic ring or heterocyclic ring;

R₃, R₄, R₇, R₈, R₉, and R₁₀ are independently selected from the groups consisting of H, halogen, hydroxyl, amino, nitro, thio, cyano, azido, C₁₋₆ alkyl, aryl-C₁₋₆ alkyl, mono- or poly-fluorinated C₁₋₆ alkyl, —C(O)R₁₃, —SO₂R₁₃, —R₁₃C(O)R₁₄, —R₁₃SO₂R₁₄, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)-amino-C₁₋₈ alkyl, nitro, thio, cyano, azido or halogen; or

any of R₃ and R₄ together, R₇ and R₈ together, or R₉ and R₁₀ together can form a 5-7 membered saturated or unsaturated cyclic ring or heterocyclic ring;

R₁ and R₁₂ is H, C₁₋₆ alkyl, aryl-C₁₋₆ alkyl, mono- or poly-fluorinated C₁₋₆ alkyl, —C(O)R₁₃, —SO₂R₁₃, —P(O)(OR₁₃)₂, —R₁₃C(O)R₁₄, —R₁₃SO₂R₁₄, —R₁₃P(O)(OR₁₄)₂, amino acid, aryl, or heterocycle; wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen; and

R₁₃ and R₁₄ are independently selected from the groups consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen; and

X is H, halogen, OH, O, SH, NH₂, NHOH, ═NOH, or NR₁₁R₁₂ wherein R₁₁ and R₁₂ are defined as above, or R₁₁ and R₁₂ together form a 5-7 membered saturated or unsaturated cyclic ring or heterocyclic ring.

Another objective of this invention is to further understand the structural features of coumarin and chromene necessary for the antiviral activity. The compounds of the present invention are useful for the study of a structure-activity relationship (SAR), in order to select and/or design other molecules for antiviral use. In addition, the instant compounds of the present invention are useful tools and/or reagents to identify and validate novel targets in the life cycle of viruses for antiviral drug development. Furthermore, the instant compounds of the present invention can be used to probe the mechanism of actions for antiviral agents.

These and other objects of the invention will become apparent in light of the detailed description below.

### DETAILED DESCRIPTION OF THE FIGURES

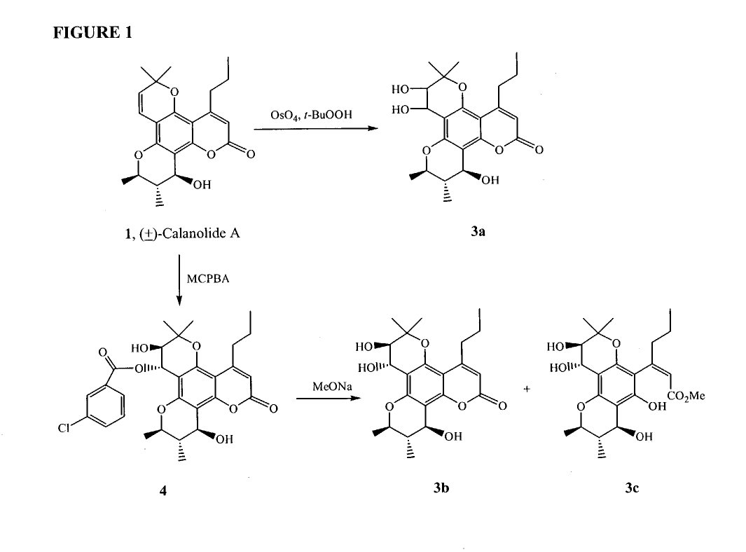

FIG. 1 is 7,8-dihydroxylation of (±)-calanolide A.

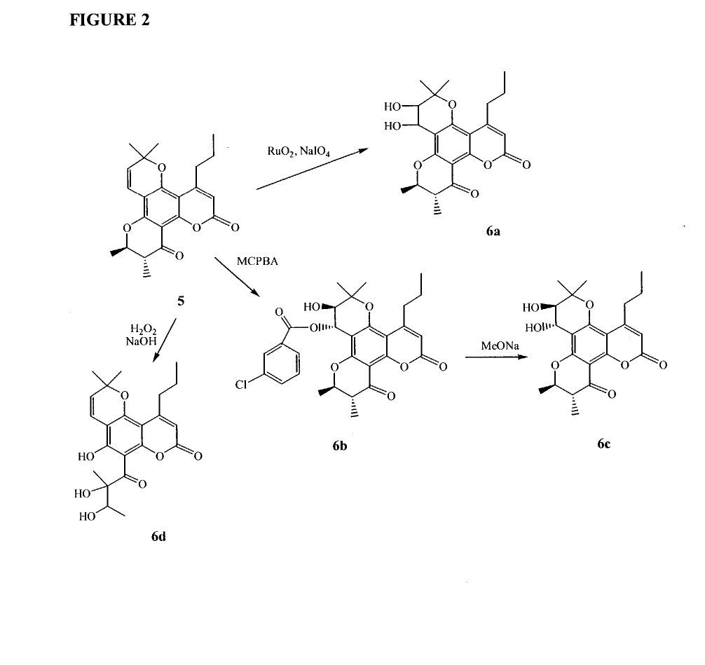

FIG. 2 is 7,8-dihydroxylation of calanolide A ketone (5).

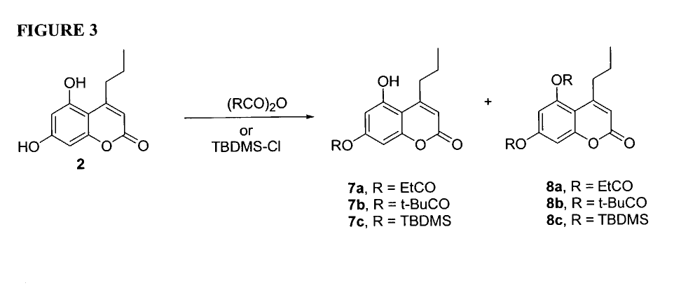

FIG. 3 illustrates acylation reactions of coumarin 2 to form compounds 7a-c and 8a-c.

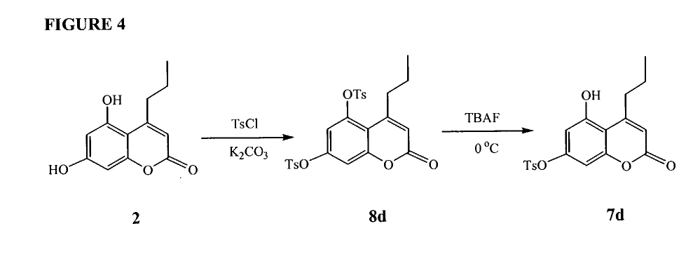

FIG. 4 shows the preparation of the tosylated coumarins 7d and 8d.

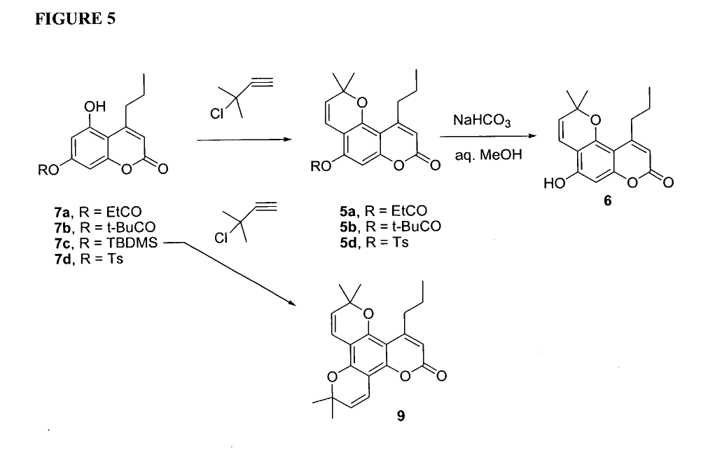

FIG. 5 illustrates the chromenylation of coumarin compounds 7a-b,d to 5a-b,d and the basic hydrolysis of acylated chromenones 5a-b,d to compound 6.

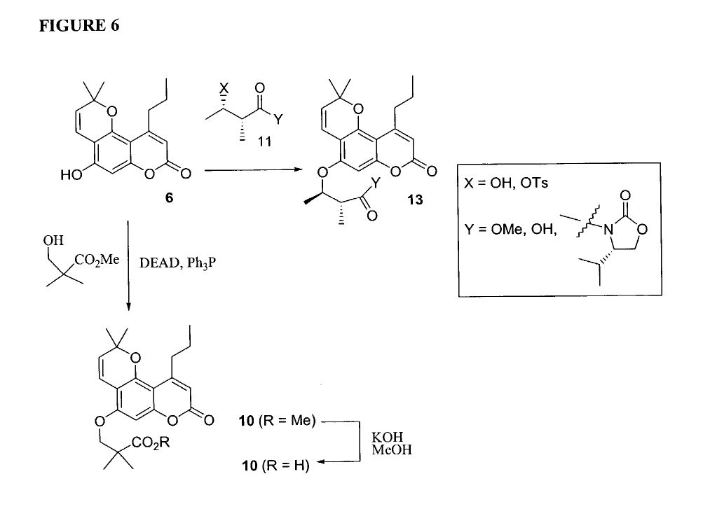

FIG. 6 illustrates the alkylation of 6 at the 7-OH.

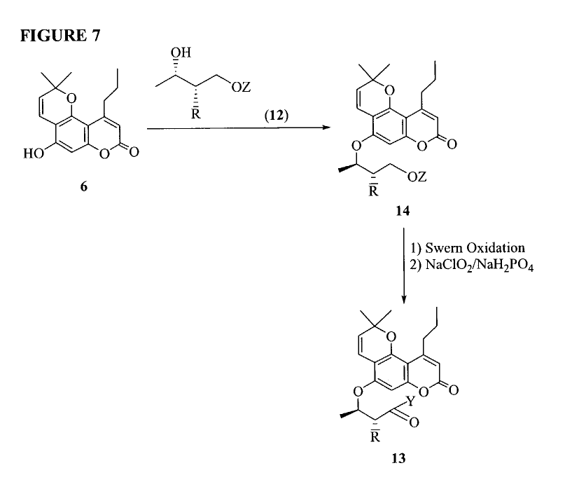

FIG. 7 further illustrates the alkylation of 6 at the 7-OH.

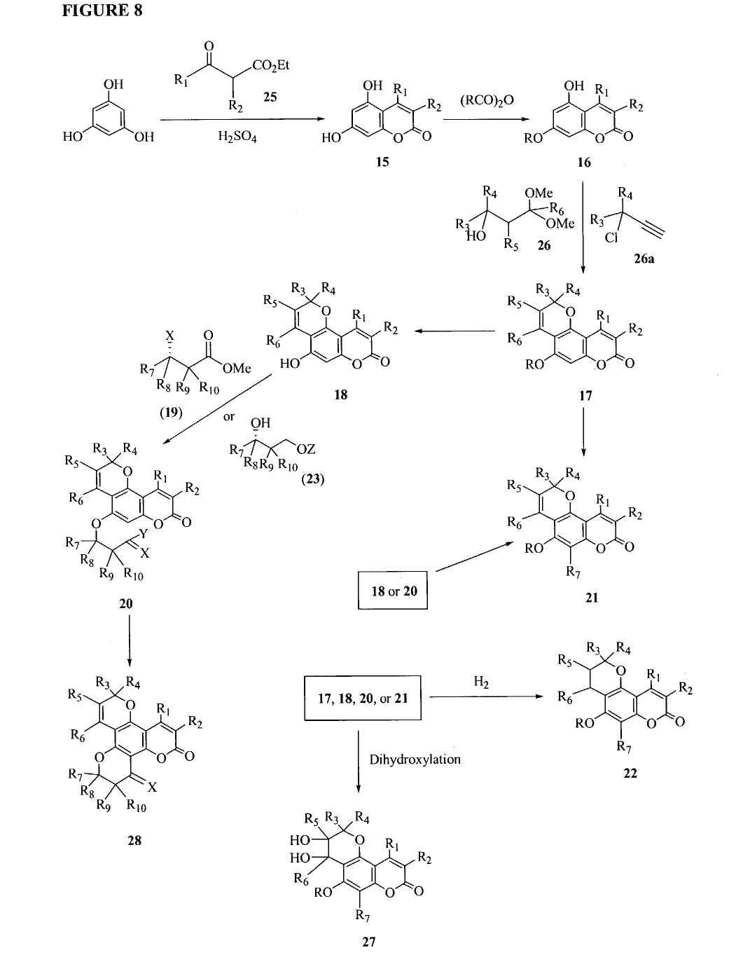

FIG. 8 illustrates the conversion of 1,3,5-trihydroxybenzene to various coumarin and chromene derivatives.

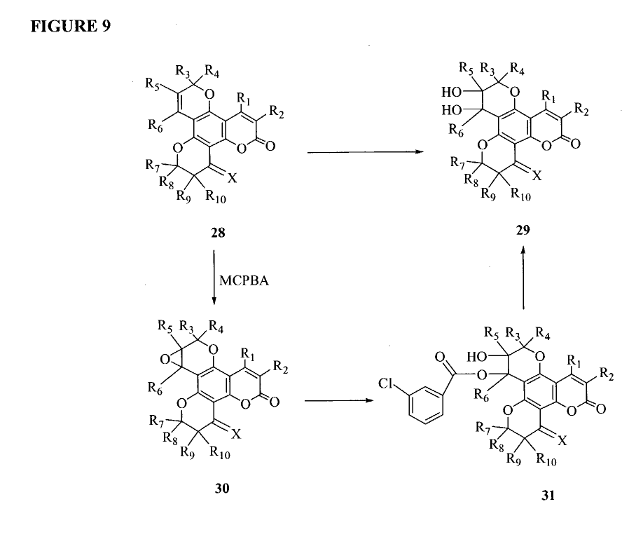

FIG. 9 illustrates the dihydroxylation of chromene compounds.

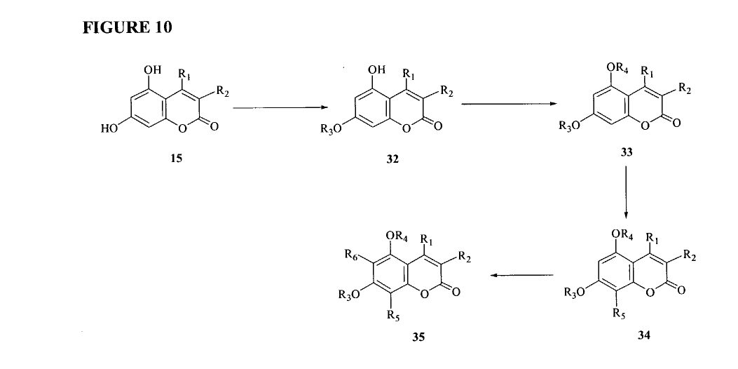

FIG. 10 illustrates the derivatization of coumarins.

### DETAILED DESCRIPTION OF THE INVENTION

The present invention relates to novel anti-viral chromene and coumarin compounds, compositions containing the same, methods of making said compounds and compositions, and their use in treating or preventing viral infections. The chromene and coumarin compounds of the instant invention encompass compounds comprising formulas I, II, and III. Chromene compounds comprise formula I:

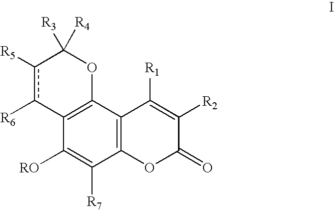

wherein R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl) amino-C₁₋₈ alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen;

R₂ is H, halogen, hydroxyl, amino, thio, cyano, C₁₋₆ alkyl, aryl-C₁₋₆ alkyl, mono- or poly-fluorinated C alkyl, aryl or heterocycle; or

R₁ and R₂ together can form a 5-7 membered saturated or unsaturated cyclic ring or heterocyclic ring;

R₃ and R₄ are independently selected from the group consisting of H, C₁₋₆ alkyl, aryl-C₁₋₆ alkyl, mono- or poly-fluorinated C₁₋₆ alkyl, aryl or heterocycle; or R₃ and R₄ together form a 5-7 membered saturated or unsaturated cyclic ring or heterocyclic ring;

R₅ and R₆ are independently selected from the groups consisting of H, halogen, hydroxyl, amino, nitro, thio, cyano, azido, C₁₋₆ alkyl, aryl-C₁₋₆ alkyl, mono- or poly-fluorinated C₁₋₆ alkyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen;

R₇ is H, halogen, hydroxyl, amino, nitro, thio, cyano, azido, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, —C(O)R₈, —SO₂R₈, —P(O)(OR₈)₂, —P(O)(OR₈)(OR₉), —R₈C(O)R₉, —R₈SO₂R₉, or R₈P(O)(OR₉)₂.

R is H, C₁₋₆ alkyl, aryl-C₁₋₆ alkyl, mono- or poly-fluorinated C₁₋₆ alkyl, aryl, heterocycle, —C(O)R₈, —SO₂R₈, —P(O)(OR₈)₂, —P(O)(OR₈)(OR₉), —R₈C(O)R₉ or —R₈SO₂R₉, —R₈P(O)(OR₉)₂, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen; and

R₈ and R₉ are independently selected from the groups consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen.

The coumarin compounds of formula II comprise:

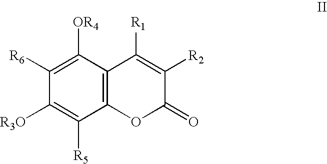

wherein

R₁ is H, halogen, hydroxyl, amino, thio, cyano, C₁-alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl) amino-C₁₋₈ alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen;

R₂ is H, halogen, hydroxyl, amino, thio, cyano, C₁₋₆ alkyl, aryl-C₁₋₆ alkyl, mono- or poly-fluorinated C₁₋₆ alkyl, aryl or heterocycle; or

R₁ and R₂ together can form a 5-7 membered saturated or unsaturated cyclic ring or heterocyclic ring;

R₃ and R₄ are independently selected from the group consisting of H, C₁₋₆ alkyl, aryl-C₁₋₆ alkyl, mono- or poly-fluorinated C₁₋₆ alkyl, aryl, heterocycle, —C(O)R₇, —SO₂R₇, —R₇C(O)R₉, P(O)(OR₇)₂, —P(O)(OR₇)(OR₈), —R₇C(O)R₈, —R₇SO₂R₈, —R₇P(O)(OR)₂, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen;

R₅ and R₆ are independently selected from the group consisting of H, halogen, hydroxyl, amino, nitro, thio, cyano, azido, C₁₋₆ alkyl, aryl-C₁₋₆ alkyl, mono- or poly-fluorinated C₁₋₆ alkyl, aryl, heterocycle, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, —C(O)R₇, —SO₂R₇, —P(O)(OR₇)₂, —P(O)(OR₇)(OR₈), —R₇C(O)R₈, —R₇SO₂R₈, or —R₇P(O)(OR₈)₂.

R₇ and R₈ are independently selected from the group consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen.

The coumarin and chromene analogues of formula III further comprise:

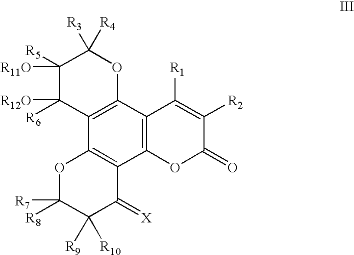

wherein

R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen;

R₂ is H, halogen, hydroxyl, amino, thio, cyano, C₁₋₆ alkyl, aryl-C₁₋₆ alkyl, mono- or poly-fluorinated C₁₋₆ alkyl, aryl or heterocycle; or

R₁ and R₂ together can form a 5-7 membered saturated or unsaturated cyclic ring or heterocyclic ring;

R₅ and R₆ are independently selected from the group consisting of H, C₁₋₆ alkyl, aryl-C₁₋₆ alkyl, mono- or poly-fluorinated C₁₋₆ alkyl, aryl or heterocycle; or R₅ and R₆ together form a 5-7 membered saturated or unsaturated cyclic ring or heterocyclic ring;

R₃, R₄, R₇, R₈, R₉, and R₁₀ are independently selected from the groups consisting of H, halogen, hydroxyl, amino, nitro, thio, cyano, azido, C₁₋₆ alkyl, aryl-C₁₋₆ alkyl, mono- or poly-fluorinated C₁₋₆ alkyl, —C(O)R₁₃, —SO₂R₁₃, —R₁₃C(O)R₁₄, —R₁₃SO₂R₁₄, aryl, or heterocycle, wherein aryl or heterocycle may each be unsubstituted or substituted with one or more of the following: C₁₋₆ alkyl, C₁₋₆ alkoxy, hydroxy-C₁₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen; or any of R₃ and R₄ together, R₇ and R₈ together, or R₉ and R₁₀ together can form a 5-7 membered saturated or unsaturated cyclic ring or heterocyclic ring;

R₁₁ and R₁₂ is H, C₁₋₆ alkyl, aryl-C₁₋₆ alkyl, mono- or poly-fluorinated C₁₋₆ alkyl, —C(O)R₁₃, —SO₂R₁₃, —P(O)(OR₁₃)₂, —R₁₃C(O)R₁₄, —R₃SO₂R₄, —R₁₃P(O)(OR₁₄)₂, amino acid, aryl, or heterocycle; wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen; and

R₁₃ and R₁₄ are independently selected from the groups consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen; and

X is H, halogen, OH, O, SH, NH₂, NHOH, ═NOH, or NR₁₁R₁₂ wherein R₁₁ and R₁₂ are defined as above, or R₁₁ and R₁₂ together form a 5-7 membered saturated or unsaturated cyclic ring or heterocyclic ring.

The present invention relates to methods for preparing novel coumain and chromene compounds and their use for treating or preventing viral infections. In one embodiment of the invention, novel coumain and chromene compounds are prepared. Some representative methods of preparation are provided herein which should not be regarded as limiting the scope or spirit of the invention. Those of skilled in the art, upon reading the instant specification, may be able to envision alternative synthetic methods.

FIG. 1 describes 7,8-dihydroxylation of (±)-calanolide A. The preparation of cis-isomers of 7,8-dihydroxy calanolide A (3a) from (±)-canalolide A (1) is straightforward using OsO₄/tBuOOH. However, treatment of 1 with MCPBA afforded the trans-hydroxyl benzoate 4. Conceivably, 4 is formed via the epoxide intermediate, followed by exclusive opening of the epoxide at the benzylic position by the benzoate. Benzoate 4 is converted to the corresponding trans-diol 3b in low yield after the treatment of NaOMe in MeOH. The lactone-opened 3c is the major product.

Similarly, 7,8-dihydroxylation of (±)-calanolide A ketone (5) is achieved (FIG. 2). Instead of OsO₄/tBuOOH, RuO₂/NaIO₄ is employed to prepare the cis-dihydroxyl compound 6a. Treatment of 5 with MCPBA afforded the trans-hydroxyl benzoate 6b, which is hydrolyzed to the corresponding trans-dihydroxyl 6c. Treatment of 5 with hydrogen peroxide and sodium hydroxide in methanol and methylene chloride yields 6d in low yield with 65% recovery of the starting material 5.

FIG. 3 shows the synthesis of mono- and bis-O-substituted coumarins as described in U.S. Pat. No. 6,369,241, which is incorporated by reference in its entirety.

In conducting this reaction, a solution of suitable acylating agent, e.g., acyl chloride or anhydride, in a suitable solvent, e.g., THF, was added in a dropwise manner to a vigorously stirred solution of 5,7-dihydroxy-4-propylcoumarin 2, a Lewis acid catalyst or a catalytic amount of a base, and an organic solvent cooled in an ice bath. Dropwise addition of the acylating agent is conducted such that the temperature of the reaction mixture is maintained at a temperature ranging between 0° C. and about 30° C.

In making compounds of the invention, the amount of acylating agent used generally ranges between about 0.5 and about 6 moles, preferably ranging between about 1 and about 2 moles, per mole of 2.

Non-limiting examples of Lewis acid catalysts useful in the acylation reaction include AlCl₃, BF₃, SnCl₄, ZnCl₂, POCl₃ and TiCl₄. A preferred Lewis acid catalyst is AlCl₃. The amount of Lewis acid catalyst relative to 5,7-dihydroxy-4-propylcoumarin, 2, ranges between about 0.5 and about 12 moles, preferably ranging between about 2 and about 5 moles, per mole of 5,7-dihydroxy-4-propylcoumarin, 2.

Non-limiting examples of a base useful in the acylation reaction include pyridine and 4-dimethylaminopyridine(DMAP). Catalytic amounts (0.1 eq) of the base may be used in combination with a suitable reaction solvent. Alternatively, the base may be used as the reaction solvent, however, complex product mixtures may results.

Non-limiting examples of organic solvent for use in the acylation reaction include THF, dichloroethane, pyridine, and mixtures thereof.

Upon completion of the addition of acylating agent, the vigorously stirred reaction mixture is maintained at a temperature ranging between about 0° C. and about 30° C., preferably about room temperature (25° C.) until the reaction reaches completion as monitored by conventional means such as TLC analysis. The reaction mixture is then poured onto ice and extracted several times with a suitable solvent such as ethyl acetate, chloroform, methylene chloride, tetrahydrofuran, or a mixture of chloroform/methanol. A preferred solvent for this extraction is ethyl acetate. The extracts are then dried over a suitable drying agent, e.g., sodium sulfate, and the product may be purified by conventional means such as silica gel column chromatography.

Compounds 7d and 8d were prepared according to the literature method with some modifications.^{45,46} Thus, tosylation of 2 with tosyl chloride and potassium carbonate led to bistosylate 8d in 90% yield. Treatment of 8d with 1.0 equiv of TBAF under mild conditions afforded 7d in 43% yield (FIG. 4).

Chromenylation of 7a was initially attempted employing 4,4-dimethyoxy-2-methylbutan-2-ol according to the literature method,^{17,48} and only ca. 5% of 5a was detected by ¹H NMR. However, when 3-chloro-3-methyl-1-butyne was used,^{49,50} 5a was obtained in 27% isolated yield (FIG. 5). The same procedure on 7b afforded 5b in 73% yield. In contrast, no 5c could be detected when 7c was reacted with 3-chloro-3-methyl-1-butyne under the same conditions. Instead, a tripyranone derivative 9⁵¹ was formed. The structure assignment of 9 was based on ¹H NMR and MS (FIG. 5). This indicated that the TBMDS-protecting group was lost during the course of chromenylation.

Hydrolysis of 5a to produce 6 under basic conditions proceeded smoothly. For example, conversion of 5a to 6 was uneventful with sodium bicarbonate in aq. MeOH in 44% yield (FIG. 5). This represents a substantial yield improvement over previous methods for preparing 6. For instance, prior reported direct chromenylation of 2 with 4,4-dimethyoxy-2-methylbutan-2-ol furnished a mixture of product, with 6 being isolated in less than 10% yield.

Alkylation of the hydroxyl group in 6 furnishes analogues with a substituent at the 7-position (FIG. 6 and FIG. 7). Various alkylating agents can be employed. For example, the introduction of the chiral side chains at the 7-position of 6 can be achieved using a variety of readily available chiral compounds 11⁵²⁻⁵⁵ and 12. The latter compound, 12 (Z=H), is resulted from reduction of 11 (X=OH, Y=OMe) with LiAlH₄. The primary OH group in 12 (Z=H) is then selectively protected such that Z is, for example, t-butyldimethylsilyl (TBDMS), tetrahydropyran (THP), p-toluenesulfonyl (Ts) or COR₁₀ wherein R₁₀ represents C₁₋₆ alkyl, aryl-C₁₋₆ alkyl, mono- or poly-fluorinated C₁₋₆ alkyl, aryl or heterocycle. According to the method, 6 is coupled to compound 11 (X=OH) under Mitsunobu conditions (PPh₃, diethyl azodicarboxylate) to provide compound 13 (FIG. 6). Compound 13 (Y≠OH) is subsequently hydrolyzed to produce 13 (Y=OH).

The reaction of 6 with 11 (X=OTs) under nucleophilic substitution conditions also generates compound 13 (FIG. 6). Hydrolysis (NaOH, LiCl) of 13 (Y=OMe), or removal of the chiral auxiliary (LiOH or LiOOH) from 13 (Y=oxazolidinone), affords the corresponding 13 (Y=OH). It should be noting that a substantial elimination from 11 (X=OH or TsO) occcured under both Mitsunobu and the nucleophilic substitution conditions, resulting in a requirment for excessive amount of the chiral moiety and a reduction in yield of 13 (FIG. 6). Alkylation of 6 with commercially available methyl 2,2-dimethyl-3-hydroxy propionate under Mitsunobu conditions furnished 10 (R=Me) in 69% yield, which was hydrolized to acid 10 (R=H) under basic conditions in 78% yield (FIG. 6).

In order to avoid the β-elimination from 11, a selectively protected chiral diol compound 12 is devised (FIG. 7). Thus, Mitsunobu reaction (PPh₃, diethyl azodicarboxylate) of 6 with 12 (Z=TBDMS) leads to the formation of 14 (Z=H), followed by removal of TBDMS protecting group (FIG. 7). No β-elimination from 12 was observed in this process. Swern oxidation of 14 (Z=H) furnishes aldehyde derivative 13 (Y=H), which is further oxidized using NaClO₂ to form the carboxylic acid, 13 (Y=OH).

The synthetic sequences described above can be extended to the synthesis of coumarin and chromene analogues (FIGS. 8, 9, and 10). Thus, Pechmann reaction of phloroglucinol with various β-ketoesters yields substituted 5,7-dihydroxycoumarin 15 (FIG. 8). Selectively protecting the 7-hydroxy group leads to the formation of 16. Chromenylation of 16 can be achieved by reacting with β-hydroxyaldehyde dimethylacetal or substituted propargyl chloride, providing chromenocoumarin 17, which is deprotected to furnish the free hydroxy group in 18. Mitsunobu reaction of 18 with 19 (X=OH), or nucleophilic substitution with 19 (X=OTs), followed by the hydrolysis, results in 20 (Y=OH). Alkylation and/or Friedel-Crafts acylation of 17, 18 or 20 provides compound 21. Hydrogenation of compound 17, 18, 20 or 21 catalyzed by any suitable catalyst, e.g., Pd/C, PtO₂, results in analogue 22. Dihydroxylation of 17, 18, 20 or 21 furnishes analogue 27 (FIG. 8). Intramolecular Friedel-Crafts cyclization or Mitsunobu reaction on 20 (Y=OH) gives chromane compounds 28. Reduction of 28 (X=O) by NaBH₄ forms the alcoholic analogues 28 (X=OH).

According to FIG. 8, 1,3,5-trihydroxybenzene was reacted with β-keto ester 25 under Pechmann conditions (See U.S. Pat. Nos. 5,489,697; 5,869,324; 5,874,591; 5,840921; 5,847,164; 5,892,060; 5,872,264; 5,981,770; 5,977,385; 6,043,271; and 6,277,879, incorporated by reference in its entirety) to produce compound 15. The amount of β-keto ester 25 to 1,3,5-trihydroxybenzene generally ranges between about 1 to about 3, preferably about 1 per mole of 1,3,5-trihydroxybenzene. β-ketoester 25 is represented by the structure:

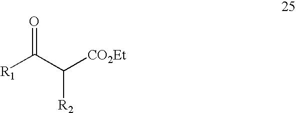

wherein R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen; R₂ is H, halogen, hydroxyl, C₁₋₆ alkyl, aryl-C₁₋₆ alkyl, mono- or poly-fluorinated C₁₋₆ alkyl, aryl or heterocycle; R₁ and R₂ together form a 5-7 membered saturated or unsaturated cyclic ring or heterocyclic ring. Compound 15 is represented by the structure:

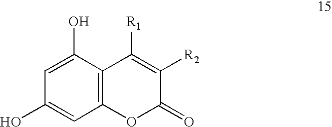

wherein R₁ and R₂ are as described above.

Thereafter, compound 15 is reacted with an acylating agent, alkylating agent, sulfonylating agent, or phosphorylating agent under conventional reation conditions to produce 16 wherein R represents C₁₋₆ alkyl, aryl-C₁₋₆ alkyl, mono- or poly-fluorinated C₁₋₆ alkyl, aryl, heterocycle, —C(O)R₇, —SO₂R₇, —R₇C(O)R₈, P(O)(OR₇)₂, —P(O)(OR₇)(OR₈), —R₇C(O)R₈, —R₇SO₂R₈, —R₇P(O)(OR₈)₂, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen. The amount of acylating agent to compound 15 generally ranges between about 0.5 to about 6, preferably about 1 per mole of 15. Compound 16 is represented by the structure:

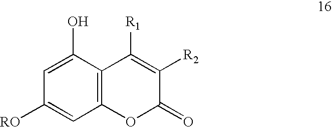

wherein R, R₁ and R₂ are as described above; and

Compound 17 is produced by chromenylation of 16 with substituted β-hydroxyaldehyde dimethylacetal 26, or substituted propargyl chloride 26a, under the reaction conditions described in U.S. Pat. Nos. 5,489,697; 5,869,324; 5,874,591; 5,840921; 5,847,164; 5,892,060; 5,872,264; 5,981,770; 5,977,385; 6,043,271; and 6,277,879, incorporated by reference in their entirety. Representative examples of substituted β-hydroxyaldehyde dimethylacetal 26 and substituted propargyl chloride 26a comprise:

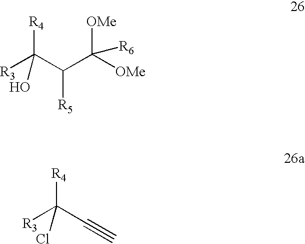

wherein R₃ and R₄ are independently selected from the group consisting of H, C₁₋₆ alkyl, aryl-C₁₋₆ alkyl, mono- or poly-fluorinated C₁₋₆ alkyl, aryl or heterocycle; or R₃ and R₄ together form a 5-7 membered saturated or unsaturated cyclic ring or heterocyclic ring; R₅ and R₆ are independently selected from the groups consisting of H, halogen, hydroxyl, amino, nitro, thio, cyano, azido, C₁₋₆ alkyl, aryl-C₁₋₆ alkyl, mono- or poly-fluorinated C₁₋₆ alkyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl-amino)C₁₋₈ alkyl, nitro, thio, cyano, azido or halogen. Compound 17 is represented by the structure:

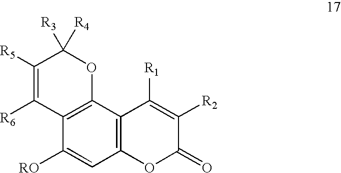

wherein R, R₁, R₂, R₃, R₄, and R₅ are as described above.

Thereafter, compound 17 (R=acyl, sulfonyl, or phosphoryl group) is hydrolyzed to produce compound 18 under the basic hydrolysis conditions described above. Compound 18 is then coupled to 19 or 23 under various conditions, e.g. Mitsunobu conditions, to produce compound 20, a representative class of 17. Compound 19 is represented by the structure:

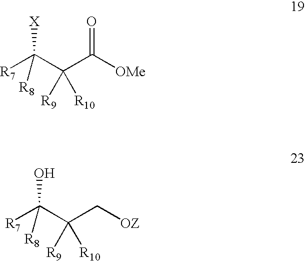

wherein R₁₄ are as described above; and X is OH, or TsO; and Z is a suitable protecting group such as TBDMS, THP, acyl, Cbz, or Boc.

Compound 20 is represented by the structure:

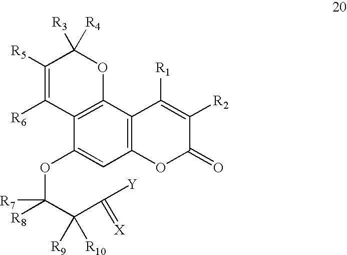

wherein R₇, R₈, R₉, and R₁₀ are independently selected from the groups consisting of H, halogen, hydroxyl, amino, nitro, thio, cyano, azido, C₁₋₆ alkyl, aryl-C₁₋₆ alkyl, mono- or poly-fluorinated C₁₋₆ alkyl, —C(O)R₁₁, —SO₂R₁₁, —R₁₁C(O)R₁₂, —R₁₁SO₂R₁₂, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)-amino-C₁₋₈ alkyl, nitro, thio, cyano, azido or halogen; and R₇ and R₈ together, or R₉ and R₁₀ together form a 5-7 membered saturated or unsaturated cyclic ring or heterocyclic ring; and R₁₁ and R₁₂ are independently selected from the groups consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each be unsubstituted or substituted with one or more of the following: C₁₋₆ alkyl, C-6 alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen; and Y represents hydrogen, OH, or OMe and X is O or H.

Intramolecular Friedel-Crafts cyclization or Mitsunobu reaction on 20 (Y=OH) gives chromane compounds 28. Reduction of 28 (X=O) by NaBH₄ forms the alcoholic analogues 28 (X=OH). The structure of 28 is represented below.

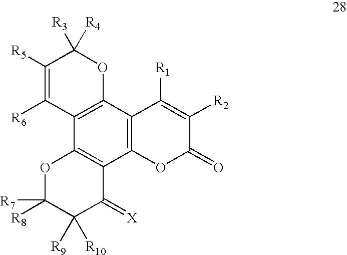

wherein R₁₋₁₀ are as described above; and X is O or OH

Alkylation or Friedel-Crafts acylation of 17, 18 or 20 under conditions described above provides compound 21 which structure is represented below.

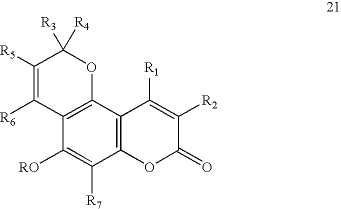

wherein R₁₋₆ are as described above; and

R₇ is H, halogen, hydroxyl, amino, nitro, thio, cyano, azido, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, —C(O)R₈, —SO₂R₈, —P(O)(OR₈)₂, —P(O)(OR₈)(OR₉), —R₈C(O)R₉, —R₈SO₂R₉, or —R₈P(O)(OR₉)₂.

R is H, C₁₋₆ alkyl, aryl-C₁₋₆ alkyl, mono- or poly-fluorinated C₁₋₆ alkyl, aryl, heterocycle, —C(O)R₈, —SO₂R₈, —P(O)(OR₈)₂, —P(O)(OR₈)(OR₉), —R₈C(O)R₉ or —R₈SO₂R₉, —R₈P(O)(OR₉)₂, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen; and

R₈ and R₉ are independently selected from the groups consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen.

Hydrogenation of compound 17, 18, 20 or 21 catalyzed by any suitable catalyst, e.g., Pd/C, PtO₂, results in calanolide analogue 22 whose structure is represented below.

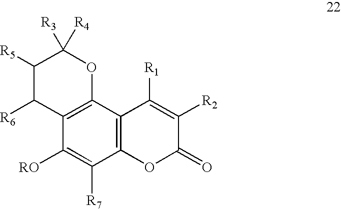

wherein R and R₁₋₇ are as described above.

Dihydroxylation of 17, 18, 20 or 21 furnishes analogue 27 whose structure is represented below.

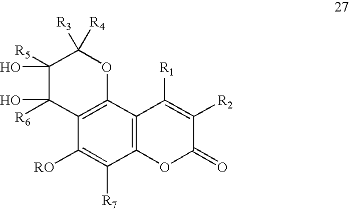

wherein R and R₁₋₇ are as described above.

According to FIG. 9, dihydroxylation of calanolide analogues 28 (See U.S. Pat. Nos. 5,489,697; 5,840,921; 5,847,164; 5,859,050; 5,869,324; 5,872,264; 5,874,591; 5,892,060; 5,977,385; 5,981,770; 6,043,271; and 6,277,879, incorporated by reference in their entirety) furnishes analogue 29 with or without formation of the intermediates 30 and 31. The structure of 29 is represented below.

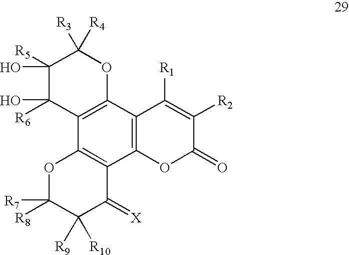

wherein R₁₋₁₀ are as described above or as defined in the references cited above and X is H, halogen, OH, O, SH, NH₂, NHOH, ═NOH, or NR₁₁R₁₂ wherein R₁₁ and R₁₂ are defined as above, or R₁₁ and R₁₂ together form a 5-7 membered saturated or unsaturated cyclic ring or heterocyclic ring.

According to FIG. 10, sequential and selective alkylation or acylation of 5,7-dihydroxycoumarin 15 (See U.S. Pat. Nos. 5,489,697; 5,840,921; 5,847,164; 5,859,050; 5,869,324; 5,872,264; 5,874,591; 5,892,060; 5,977,385; 5,981,770; 6,043,271; 6,277,879; 6,369,241, as well as WO 00/64902, and WO 00/64903, incorporated by reference in their entirety) affords coumarins with a variety of substituents (35), represented by the structure below.

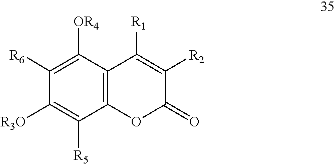

wherein

R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen;

R₂ is H, halogen, hydroxyl, amino, thio, cyano, C₁₋₆ alkyl, aryl-C₁₋₆ alkyl, mono- or poly-fluorinated C₁₋₆ alkyl, aryl or heterocycle; or

R₁ and R₂ together can form a 5-7 membered saturated or unsaturated cyclic ring or heterocyclic ring;

R₃ and R₄ are independently selected from the group consisting of H, C₁₋₆ alkyl, aryl-C₁₋₆ alkyl, mono- or poly-fluorinated C₁₋₆ alkyl, aryl, heterocycle, —C(O)R₇, —SO₂R₇, —P(O)(OR₇)₂, —P(O)(OR₇)(OR₈), —R₇C(O)R₈ or —R₇SO₂R₈, —R₇P(O)(OR₈)₂, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen;

R₅ and R₆ are independently selected from the group consisting of H, halogen, hydroxyl, amino, nitro, thio, cyano, azido, C₁₋₆ alkyl, aryl-C₁₋₆ alkyl, mono- or poly-fluorinated C₁₋₆ alkyl, aryl, heterocycle, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, —C(O)R₇, —SO₂R₇, —P(O)(OR₇)₂, —P(O)(OR₇)(OR₈), —R₇C(O)R₈, —R₇SO₂R₈, or —R₇P(O)(OR₈)₂;

R₇ and R₈ are independently selected from the group consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each be unsubstituted or substituted with one or more of the following: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido or halogen.

Definitions

Except as expressly defined otherwise, the following definition of terms is employed throughout this specification.

The terms “alkyl”, “lower alkyl” or “C₁₋ₙ alkyl” mean a straight or branched hydrocarbon having from 1 to n carbon atoms and includes, for example, methyl, ethyl, n-propyl, isopropyl, n-butyl, sec-butyl, isobutyl, tert-butyl, n-pentyl, n-hexyl, and the like. The alkyl group can also be substituted with one or more of the substituents listed below for aryl.

By “alkoxy”, “lower alkoxy” or “C₁₋ₙ alkoxy” in the present invention is meant straight or branched chain alkoxy groups having 1-n carbon atoms, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

The term “halogen” includes chlorine, fluorine, bromine, and iodine, and their monovalent radicals.

The term “aryl” means an aromatic carbocyclic group having a single ring (e.g., phenyl), multiple rings (e.g., biphenyl), or multiple condensed rings in which at least one is aromatic (e.g., 1,2,3,4-tetrahydronaphthyl, naphthyl, anthracyl, or phenanthryl), unsubstituted or substituted by 1 to 3 substituents selected from alkyl, O-alkyl and S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH alkyl, —N(alkyl)₂, —CH₂)ₘPO₃H₂, —(CH₂)ₘPO₃(alkyl)₂, —(CH₂),SO₂NH₂, and —(CH₂)ₘSO₂NH-alkyl wherein alkyl is defined as above and m is 0, 1, 2, or 3.

The term “cyclic ring” as referred to herein means a monocyclic or polycyclic moiety. By “polycyclic” is meant two or more rings that share two or more carbon atoms. A “carbocyclic group” which contains hetero atoms as one or more of its members can be referred to as a “heterocycle” or a “heterocyclic ring”. Such a “heterocycle” can likewise be “monocyclic” or “polycyclic”. A cyclic ring and a heterocyclic ring can be saturated, can contain one or more double bonds or can be aromatic. Each ring can be unsubstituted or substituted by 1 to 3 substituents selected from the group as described above for aryl.

In another embodiment, the invention provides methods for treating or preventing viral infections in a subject comprising the use of compounds of formula I, II or III. Examples of subjects include mammals, such as, for example, humans, primates, bovines, ovines, porcines, felines, canines, etc. Examples of viruses can include, but are not limited to, HIV-1, HIV-2, herpes simplex virus (type 1 and 2) (HSV-1 and 2), varicella zoster virus (VZV), cytomegalovirus (CMV), papilloma virus, HTLV-1, HTLV-2, feline leukemia virus (FLV), Epstein Barr virus, avian sarcoma viruses such as rous sarcoma virus (RSV), hepatitis types A-E, equine infections, influenza A and B virus, parainfluenza, adenovirus, arboviruses, respiratory syncytial virus, measles, mumps and rubella viruses. More preferably the methods of the present invention are used to treat a human infected with HIV, Hepatitis B, cytomegalovirus, Epstein Barr virus, or measles.

In another embodiment, the invention provides use of the compounds of formula I, II, or III for the manufacture of a medicament for treating or preventing viral infections, such as those viral infections related to the non-limiting examples of the viruses described above.

Hence the compounds of the present invention are particularly useful in the prevention or treatment of infection by the human immunodeficiency virus and also in the treatment of consequent pathological conditions associated with AIDS. Treating AIDS is defined as including, but not limited to, treating a wide range of states of HIV infection: AIDS, ARC, both symptomatic and asymptomatic, and actual or potential exposure to HIV. For example, the compounds of this invention are useful in treating infection of HIV after suspected exposure to HIV by e.g., blood transfusion, exposure to patient blood during surgery or an accidental needle stick.

Antiviral compounds of the invention may be formulated as a solution of lyophilized powders for parenteral administration. Powders may be reconstituted by addition of a suitable diluent or other pharmaceutically acceptable carrier prior to use. The liquid formulation is generally a buffered, isotonic, aqueous solution. Examples of suitable diluents are normal isotonic saline solution, standard 5% dextrose in water or in buffered sodium or ammonium acetate solution. Such formulation is especially suitable for parenteral administration, but may also be used for oral administration. It may be desirable to add excipients such as polyvinylpyrrolidone, gelatin, hydroxy cellulose, acacia, polyethylene glycol, mannitol, sodium chloride or sodium citrate.

Alternatively, the compounds of the present invention may be encapsulated, tableted or prepared in an emulsion (oil-in-water or water-in-oil) or syrup for oral administration. Pharmaceutically acceptable solids or liquid carriers, which are generally known in the pharmaceutical formulary arts, may be added to enhance or stabilize the composition, or to facilitate preparation of the composition. Solid carriers include starch (corn or potato), lactose, calcium sulfate dihydrate, terra alba, croscarmellose sodium, magnesium stearate or stearic acid, talc, pectin, acacia, agar, gelatin, maltodextrins and microcrystalline cellulose, or colloidal silicon dioxide. Liquid carriers include syrup, peanut oil, olive oil, corn oil, sesame oil, saline and water. The carrier may also include a sustained release material such as glyceryl monostearate or glyceryl distearate, alone or with a wax. The amount of solid carrier varies but, preferably, will be between about 10 mg to about 1 g per dosage unit.

The dosage ranges for administration of antiviral compounds of the invention are those to produce the desired affect whereby symptoms of infection are ameliorated, slowed, or prevented from further progression. For example, as used herein, a pharmaceutically effective amount for an HIV or other viral infection refers to the amount administered so as to maintain an amount which suppresses or inhibits secondary infection by syncytia formation or by circulating virus throughout the period during which the HIV or other viral infection is evidenced, such as by presence of antiviral antibodies, presence of culturable virus and presence of antigen in patient sera. For example, the presence of anti-HIV antibodies can be determined through use of standard ELISA or Western blot assays, e.g., anti-gp120, anti-gp41, anti-tat, anti-p55, anti-p17, antibodies, etc. The dosage will generally vary with age, extent of the infection, the body weight and counterindications, if any, for example, immune tolerance. The dosage will also be determined by the existence of any adverse side effects that may accompany the compounds. It is always desirable, whenever possible, to keep adverse side effects to a minimum.

One skilled in the art can easily determine the appropriate dosage, schedule, and method of administration for the exact formulation of the composition being used in order to achieve the desired effective concentration in the individual patient. However, the dosage can vary from between about 0.001 mg/kg/day to about 50 mg/kg/day, but preferably between about 0.01 to about 20 mg/kg/day.

For viral infections other than HIV, antiviral activity can be shown via other standard assays. For example, antiviral efficacy against HSV, CMV, and VZV can be determined by cytopathic effect (CPE) inhibition assay. Similarly, efficacy against HSV-1, HSV-2, VZV, CMV can be determined by plaque reduction assay. In this method, the reduction of plaque on a treated agar plate is compared to an untreated control. Efficacy against EBV can be determined by immunofluoresence assay, where monoclonal antibodies and fluorescin conjugated anti-mouse antibody are sequentially added to incubated cell cultures infected with EBV, with the number of fluoresence positive cells in smears ultimately counted.

The pharmaceutical composition may contain other pharmaceuticals in conjunction with the antiviral compounds of the invention. For example, other pharmaceuticals may include, but are not limited to, other antiviral compounds (e.g., AZT, ddC, ddI, D4T, 3TC, acyclovir, gancyclovir, fluorinated nucleosides and nonnucleoside analog compounds such as TIBO derivatives and nevirapine, α-interfon and recombinant CD4), protease inhibitors (e.g., indinavir, saquinavir, ritonavir, and nelfinavir), immunostimulants (e.g., various interleukins and cytokines), immunomodulators, antibiotics (e.g., antibacterial, antifungal, anti-pneumocysitis agents), and chemokine inhibitors. Administration of the inhibitory compounds with other anti-retroviral agents that act against other HIV proteins such as protease, intergrase and TAT will generally inhibit most or all replicative stages of the viral life cycle. The other pharmaceuticals may be formulated together with the antiviral compounds of the invention into the same pharmaceutical products.

The antiviral compounds described herein can be used either alone or in conjunction with other pharmaceutical compounds to effectively combat a single infection. For example, the compounds of the invention can be used either alone or combined with acyclovir in a combination therapy to treat HSV-1. The compounds can also be used either alone or in conjunction with other pharmaceutical compounds to combat multiple infections. For example, the antiviral compounds can be used in combination with Intron A and/or a biflavanoid for treating Hepatitis B; with gancyclovir, progancyclovir, famcyclovir, foscarnet, vidarabine, cidovir, and/or acyclovir for treating herpes viruses; and with ribavarin, amantidine, and/or rimantidine for treating respiratory viruses.

In addition, the compounds of the present invention are useful as tools and/or reagents to study inhibition of retroviral reverse transcriptases. For example, the instant compounds selectively inhibit HIV reverse transcriptase. Hence, the instant compounds are useful as a structure/activity relationship (SAR) tool to study, select and/or design other molecules to inhibit HIV.

The following examples are illustrative and do not serve to limit the scope or sprit of the invention, as claimed. The inhibitory activities against HIV and other viruses including hepatitis B, herpes viruses (HSV-1, HSV-2, HCMV, VZV, and Epstein Barr virus), and respiratory viruses (influenza A, influenza B, parainfluenza, adenovirus, measles, and respiratory syncytial virus) were investigated.

Experimental Section

General: Melting points were uncorrected. All commercial reagents and solvents were used without further purification. The ¹H NMR (300 MHz) and ¹³C NMR (75 MHz) were run in indicated deuterated solvent and chemical shifts are reported in ppm with tetramethylsilane as the internal standard.

### EXAMPLE 1

Reaction of (±)-calanolide A (1) with t-BuOOH catalyzed by OsO₄. The formation of cis-7,8-Dihydroxy calanolide A (3a):

To a round-bottom flask, equipped with magnetic stirrer, were added acetone (10 mL), calanolide A (1, 0.5 g, 1.35 mmol), t-butylammnium acetate (88.4 mg, 0.34 mmol), and t-butylhydroperoxide (70%, 0.3 mL, 2.19 mmol) as one portion. The solution was stirred at room temperature until a homogeneous solution was obtained. The temperature of the solution was lowered to 0° C. A solution of OsO₄ (1 mg, 0.004 mmol) in t-butyl alcohol was added. The reaction mixture turned purple. After 1 hour, the cold bath was removed. The reaction mixture was warmed up to room temperature and stirred for 30 hours. Ether (10 mL) was added, and the resulting mixture was cooled to 0° C., followed by addition of freshly prepared 10% aqueous NaHSO₃ (15 mL). The cold bath was removed and stirring was continued for another hour to afford a two-layer solution. Solid NaCl was added to the aqueous layer until it was saturated. After the two-phase solution was stirred for 10 minutes, the organic layer was separated and the aqueous phase was extracted with ether (3×10 mL). The combined organic phase was dried with brine and concentrated to give a brown residue as the crude product. The crude product was separated via 2 mm-Chromatotron with ethyl acetate/hexane (25%) as the eluent to afford compund cis-3a (130.1 mg, 23.8%). ¹H NMR (400 MHz, CDCl₃) δ 5.98 (s, 1H), 5.03 (m), 4.76 (m), 4.75 (m), 4.13 (m), 3.91 (d, J=2.0 Hz, 1H), 3.81 (m, 1H), 3.59 (m, 1H), 3.2 (m), 2.86 (m), 1.62 (m), 1.50 (m), 1.40 (d, J=9.2 Hz), 1.26 (t, J=7.2 Hz, 3H), 1.16 (m), 1.02 (t, J=7.2 Hz, 3H); ¹³C NMR (100.5 MHz, CDCl₃) δ 177.16, 160.32, 159.31, 155.97, 155.00, 151.06, 110.89, 110.77, 110.71, 109.77, 106.34, 106.10, 104.39, 79.07, 78.93, 78.05, 69.95, 66.56, 62.44, 39.98, 39.08, 38.96, 31.91, 30.92, 29.64, 29.35, 24.77, 24.49, 24.00, 23.23, 22.63, 22.07, 21.92, 21.69, 21.04, 19.26, 19.21, 19.08, 15.21, 15.03, 14.18, 13.92, 12.86, 12.57; MS 404.5 (405.2 found); elemental analysis: C 65.33% (65.54% found), H 6.98% (7.20% found).

### EXAMPLE 2

Reaction of (±)-calanolide A (1) with m-chloroperbenzoic Acid (MCPBA). The Formation of Benzoate 4:

To a solution of calanolide A (1, 0.4 g, 1.1 mmol) in CH₂Cl₂ (2 mL) at 0° C. was added a solution of MCPBA (0.5 g, 2.9 mmol). The reaction mixture was stirred at 0° C. for 3 hours. The reaction was quenched with 10% aqueous sodium sulfite solution (15 mL), followed by saturated NaHCO₃ solution (5 mL). The resulting two layers were separated and the aqueous layer was extracted with CH₂Cl₂ (3×5 mL). The combined organic phase was washed with brine and dried over Na₂SO₄. Concentration under reduced pressure yielded a yellow residue as the crude product. The residue was purified by 2-mm chromatotron with ethyl acetate/hexane (1:3) as the eluent to afford the benzoate 4 (178.6 mg, 46.1%): ¹H NMR (300 MHz, CDCl₃) δ 7.97 (m, 1H), 7.88 (m, 1H), 7.56 (m, 1H), 7.38 (m, 1H), 6.15 (d, J=4.5 Hz, 1H), 6.00 (s, 1H), 4.71 (d, J=7.8 Hz, 1H), 4.12 (m, 1H), 3.96 (t, J=4.8 Hz, 1H), 3.84 (m, 1H), 3.59 (s, 1H), 2.98 (m, 2H), 1.67 (m, 1H), 1.57 (m, 6H), 1.49 (m, 3H), 1.10 (d, J=3.0 Hz, 3H), 1.05 (t, J=7.2 Hz, 3H); ¹³C NMR (75.5 MHz, CDCl₃) δ 165.77, 160.32, 159.28, 157.07, 155.93, 152.01, 134.75, 133.39, 131.60, 129.88, 129.85, 127.91, 110.56, 07.00, 104.04, 103.27, 79.19, 72.58, 71.64,69.19, 66.98, 66.80, 65.15, 53.36, 40.37, 40.25, 38.91, 31.82, 29.60, 29.55, 29.26, 26.10, 24.15, 23.18, 22.57, 21.82, 19.84, 18.54, 18.50, 14.79, 14.70, 13.98, 13.81, 12.19; MS 543.0 (543.2 found); Elemental analysis C 64.14% (63.82% found), H 5.75% (5.95% found).

### EXAMPLE 3

Hydrolysis of Benzoate Derivative 4:

To a solution of benzoate 4 (1.2 g, 2.2 mmol) was added a solution of sodium methoxide in MeOH (15 mL) prepared by addition of a small piece of sodium to MeOH. The solution was stirred at room temperature for three days. The solvent was removed under reduced pressure to give a yellow solid. The solid was separated by chromatography with ethyl acetate (25% to 40% gradient) as the eluent. Compound 3c (275.7 mg, 28.9%) was collected first followed by trans-3b (69.8 mg, 7.8%).

3c: ¹H NMR (400 MHz, CDCl₃) δ 5.96(s, 1H), 4.77(m), 4.51(d, J=4.8 Hz), 4.46 (d, J=4.8 Hz), 4.04(m), 3.89(m), 366(s), 3.63(s), 3.55(m), 1.98(m), 1.63(m), 1.49(m), 1.17(d, J=1.2 Hz), 1.16 (d, J=1.2 Hz), 1.01 (t, J=7.6 Hz); ¹³C NMR (100.5 MHz, CDCl₃) δ 160.52, 160.50, 159.38, 157.35, 156.87, 155.46, 155.38, 151.64, 151.45, 110.24, 106.45, 106.37, 105.75, 104.00, 103.76, 79.08, 78.95, 77.45, 71.21, 71.18, 71.14, 70.66, 67.25, 67.10, 60.62, 60.01, 40.61, 40.32, 39.08, 39.05, 26.52, 26.06, 23.25, 23.22, 21.48, 20.62, 18.92, 15.09, 14.94, 13.92; MS 436.4 (434.3 found).

trans-3b: ¹H NMR (400 MHz, CDCl₃) δ 5.98 (s, 1H), 5.03 (m), 4.75 (m), 4.76 (m), 4.12 (m), 3.92 (m, 1H), 3.80 (m, 1H), 3.61 (m), 3.22 (d, J=4.4 Hz), 2.93 (m), 2.83 (m), 1.98 (m), 1.63 (m), 1.50 (m), 1.15 (d, J=6.8 Hz, 3H), 1.02 (t, J=6.8 Hz, 3H); ¹³C NMR (75.5 MHz, CDCl₃) δ 160.43, 19.37, 156.00, 154.99, 151.04, 110.71, 106.36, 106.16, 104.36, 78.94, 70.00, 66.50, 62.40, 40.00, 39.07, 24.09, 23.23, 22.56, 19.24, 15.21, 13.91; MS 404.5 (405.2 found).

### EXAMPLE 4

Reaction of trans-ketone 5 with NaIO₄ Catalyzed by RuO₂. The Formation of 6a:

To a vigorously stirred solution of trans-ketone 5 (1.0 g, 2.7 mmol) in ethyl acetate/acetonitrile (1:1, 16 mL) at 0° C. was added a solution of RuO₂ (42.7 mg, 0.2 mmol) and NaIO₄ (1.8667 g, 14.0 mmol) in distilled water as one portion. The resulting solution was stirred vigorously at 0° C. for 3.5 hours and turned brown. After saturated Na₂SO₃ solution (25 mL) was added, the reaction mixture was separated and the aqueous phase was extracted with ethyl acetate (5×15 mL). The combined organic phase was dried over Na₂SO₄ and concentrated to give a black residue. The residue was dissolved in ethyl acetate (10 mL) and passed through a short silica gel column (5 cm) with ethyl acetate as the eluent. The collected solution was concentrated to afford a brown solid which was subsequently purified via 2-mm Chromatotron with ethyl acetate/hexane (1:2) as the eluent to give compound cis-6a (91.3 mg, 8.4%): ¹H NMR (400 MHz, CDCl₃) δ 6.05 (s, 1H), 5.04 (s, 1H), 4.63 (m), 4.47 (m), 3.85 (m), 3.66 (d, J=2.8 Hz, 1H), 3.22 (m, 1H), 2.86 (m), 2.76 (m), 1.61 (m), 1.54 (m), 1.45 (d, J=7.6 Hz), 1.24 (t, J=7.2 Hz, 3H), 1.01 (t, J=7.2 Hz, 3H); ¹³C NMR (75.5 MHz, CDCl₃) δ 189.52, 163.16, 162.91, 159.92, 59.89, 157.78, 157.76, 156.78, 157.76, 156.09, 155.98, 155.80, 112.41, 112.37, 106.65, 106.28, 104.68, 103.57, 80.36, 80.29, 80.23, 70.20, 70.02, 61.65, 53.36, 47.15, 47.03, 39.01, 31.45, 24.87, 24.64, 23.02, 22.50, 21.97, 21.62, 19.73, 19.64, 13.95, 13.71, 10.55, 10.08; MS 402.4 (403.3 found); elemental analysis: C 65.66% (65.50% found), H 6.51% (6.56% found).

### EXAMPLE 5

Reaction of Trans-Ketone 5 with m-chloroperbenzoic Acid (MCPBA). The Formation of the Benzoate 6b:

To a solution of trans-ketone 5 (0.40 g, 1.1 mmol) in CH₂Cl₂ (2 mL) at 0° C. was added a solution of MCPBA. The reaction mixture was stirred at 0° C. for 3 hours. The reaction was quenched with 10% aqueous sodium sulfite solution (15 mL), followed by saturated NaHCO₃ solution (5 mL). The resulting two layers were separated and the aqueous layer was extracted with CH₂Cl₂ (3×5 mL). The combined organic phase was washed with brine and dried over Na₂SO₄. Concentration under reduced pressure yielded a yellow residue as the crude product. The residue was purified by 2-mm chromatotron with ethyl acetate/hexane (1:3) as the eluent to give compound benzoate 6b as a white solid (352.5 mg, 59.2%): ¹H NMR (300 MHz, CDCl₃) δ 7.98 (m, 1H), 7.91 (m, 1H), 7.60 (m, 1H), 7.54 (m, 1H), 6.48 (m, 1H), 6.08 (s, 1H), 4.25 (m, 1H), 4.10 (m, 2H), 4.01 (m, 1H), 2.92 (m, 2H), 2.31 (m, 1H), 1.66 (m, 2H), 1.54 (m, 6H), 1.25 (m, 3H), 1.14 (d, J=6.0 Hz, 3H), 1.02 (t, J=7.5 Hz, 3H); %): ¹³C NMR (100.5 MHz, CDCl₃) δ 189.48, 165.11, 164.97, 163.49, 163.20, 159.78, 159.73, 159.66, 157.51, 157.45, 157.42, 156.81, 156.73, 156.67, 156.45, 156.25, 134.79, 134.66, 133.50, 133.46, 133.35, 131.38, 131.33, 129.96, 129.97, 129.87, 129.70, 129.62, 129.54, 128.85, 127.81, 127.74, 127.66, 15.96, 112.28, 112.19, 104.45, 103.84, 103.70, 102.60, 80.46, 80.32, 80.20, 80.17, 80.01, 71.96, 71.58, 68.33, 68.05, 47.03, 47.00, 39.05, 30.90, 26.17, 24.42, 24.06, 23.19, 23.17, 22.64, 22.12, 20.61, 19.32, 19.30, 13.87, 10.29, 10.26, 9.97; MS 541.0 (541.6 found).

### EXAMPLE 6

Hydrolysis of Meta-chlorobenzoate 6b. The Formation of 6c:

To a solution of benzoate 6b (0.3 g, 0.55 mmol) in MeOH (12 mL) was added a solution of KOH (2.1 mg, 2.30 mmol) in water (3 mL). After stirring at room temperature for 10 minutes, the solution was concentrated. Water (15 mL) was added to form a yellow cloudy solution. The pH value of the solution was adjusted to 2 with 1N HCl solution to give a white precipitate. Ethyl acetate (15 mL) was added to dissolve the precipitate, and a two-layer solution was formed. The solution was separated and the aqueous phase was extracted with ethyl acetate (3×10 mL). The organic extracts were combined and washed with brine and dried over Na₂SO₄. Concentration of the solution afforded a yellow solid. The solid was purified via 2-mm Chromatotron to afford compound 6c as a white solid (14.2 mg, 5.9%). ¹H NMR (400 MHz, CDCl₃) δ 6.04 (s, 1H), 4.55 (d, J=5.2 Hz, 11H), 4.50 (d, J=5.2 Hz, 11H), 4.40 (m), 4.28 (m), 3.84 (m), 3.70 (s), 3.66 (s), 2.88 (m), 2.63 (m), 2.55 (m), 1.61 (m), 1.50 (m), 1.25 (m), 1.01 (t, J=6.8 Hz, 3H); ¹³C NMR (75.5 MHz, CDCl₃) δ 189.82, 163.65, 159.92, 157.70, 156.40, 112.52, 105.36, 80.60, 80.47, 80.12, 71.37, 71.09, 70.67, 70.32, 60.82, 60.18, 47.37, 47.34, 39.36, 29.91, 26.23, 26.09, 23.38, 22.91, 22.02, 21.47, 19.94, 19.87, 14.33, 14.10, 10.78, 10.41; MS 434.5 (433.9 found).

### EXAMPLE 7

Reaction of Trans-Ketone 5 with H₂O₂. The Formation of 6d:

To a solution of trans-ketone 5 (0.50 g, 1.3 mmol) in CH₂Cl₂ (5 mL) and MeOH (15 mL) was added H₂O₂ (0.25 mL, 50% aqueous solution, 0.30 g, 4.1 mmol) at 15° C. After the temperature of the reaction mixture was stablized at 15° C., NaOH solution (2.5 mL, 0.275 M aqueous solution, 0.68 mmol) was added dropwise. After the yellow solution was stirred at 15° C. for 24 hours, water (20 mL) and ether (15 mL) were added subsequently. The two layers were separated and the aqueous layer was further extracted with ether (3×15 mL). The combined organic phase was dried over Na₂SO₄ and concentrated to give a yellow residue. The residue was purified by column chromatography with ethyl acetate/hexane (1:9) as the mobile phase to yield 320 mg starting material and 52 mg of an unidentified compound.

The aqueous phase from the above extraction was acidified with concentrate HCl solution until the pH value was around 3. After ether (3×10 mL) extractions, the combined organic solution was concentrated to afford compound 6d (66.9 mg, 13% yield) as an orange solid: ¹H NMR (300 MHz, CDCl₃) δ 6.62 (d, J=10.2 Hz, 1H), 6.04 (s, 1H), 5.61 (d, J=9.9 Hz, 1H), 4.89 (m, 1H), 2.92 (m, 2H), 1.66 (m, 2H), 1.46 (s, 3H), 1.44 (m, 3H), 1.32 (d, J=7.2 Hz, 3H), 1.23 (d, J=6.0 Hz, 3H), 1.03 (t, J=7.2 Hz, 3H); ¹³C NMR (75.5 MHz, CDCl₃) δ 179.32, 161.68, 159.57, 143.45, 143.21, 142.29, 131.68, 129.06, 117.64, 112.57, 111.55, 106.59, 78.75, 45.45, 38.42, 27.28, 27.22, 23.00, 16.17, 13.84, 13.13; MS 402.4 (403.2 found).

### EXAMPLE 8

Determination of Acylation Conditions for the Conversion of 2 to 7a

Due to the low yields from the Friedel-Crafts acylation reaction, a more practical procedure needed to be developed for the synthesis of 7a from 2. A variety of reaction conditions were investigated, which are summarized in Table I. Without Lewis acid such as AlCl₃ or base such as pyridine and 4-dimethylaminopyridine (DMAP), no reaction took place between coumarin 2 and propionyl anhydride (entries 1 and 2 in Table I). The reported conditions using AlCl₃⁴⁵ were repeated and led to 20% conversion of 2 to 7a, as indicated by HPLC analysis (entry 3). The best results were obtained when a catalytic amount of pyridine was used (entry 4), with 47% conversion to 7a along with a small amount of undesired diester 8a and some unreacted starting material 2. If pyridine was used as the solvent, a complex mixture of products was formed. The formation of the undesired diester 8a could be minimized by shortening the reaction time or lowering the reaction temperature, which, however, could also decrease the yield of the desired product 7a with increasing of the unreacted starting 2 (entry 5). On the other hand, prolonged reaction time or increasing the reaction temperature would increase the conversion of the starting material 2 to 7a, however, this was accompanied by an increase in the formation of undesired 8a and led to a more difficulty in purification of 7a. It appeared that DMAP might be too strong a base (entries 6 and 7) and proprionyl chloride too reactive an acylating agent (entry 8) for the selective acylation. Therefore, the pyridine promoted acylation was scaled up in a 50-gram scale reaction, affording 36% isolated yield of 7a. For introduction of a more bulky group at the 7-position of 2, a more reactive acylating agent such as acyl chloride can be used. For example, reaction between 2 and pivaloyl chloride at r.t. in the presence of pyridine yielded 7-monosubstituted 7b and 5,7-disubstituted 8b in isolated yields of 36% and 18%, respectively. It is worthwhile noting that reaction of 2 with 1.0 equivalent TBDMS-Cl in the presence of imidazole in DMF afforded 31% of 7-TBDMS substituted 7c, along with 24% of 5,7-bis(TBDMS) substituted 8c (FIG. 3). Furthermore, a mixture of approximately 1:1 of mono- and bis-tosylated compounds were obtained (7 and 8, R=Ts) when 2 was reacted with tosyl chloride in the presence of a catalytic amount of pyridine.

TABLE I
Acylation of Coumarin 2 with Propionyl
Anhydride to Form 7-monoester 7a

| | Scale | | HPLC Yield |
|---|---|---|---|
| Entry | of 2 | Reaction Conditions | of 7a |
| 1 | 2.5 g | in THF at 0° C. for 2 hr | no reaction |
| 2 | 2.5 g | in THF at 30° C. for 2 hr | no reaction |
| 3 | 5 g | AlCl₃ (2 eq.) in 1,2-dichloroethane at r.t | 20% |
| | | for 24 hr | |
| 4 | 2.5 g | pyridine (3 drops) in THF at r.t for 1.5 hr | 47% |
| 5 | 2.5 g | pyridine (3 drops) in THF at r.t for 1 hr | 26% |
| 6 | 5 g | 4-dimethylaminopyridine (0.1 eq.) in 1,2- | complicated |
| | | dichloroethane at r.t for 4 hr | results |
| 7 | 5 g | 4-dimethylaminopyridine (0.1 eq.) in 1,2- | 47% isolated |
| | | dichloroethane at 0° C. for 45 min | yield of 8a |
| 8 | 5 g | propionyl chloride in pyridine at 0° C. for | complicated |
| | | 4 hrs | results |

### EXAMPLE 9

5-Hydroxy-7-propionyloxy-4-propylcoumarin (7a):

To a 2 L three-neck round-bottom flask equipped with a stir bar, additional funnel, N₂ inlet and outlet were added 50 g (2.27 mol) of 5,7-dihydroxy-4-propylcoumarin (2) and 500 mL of anhydrous THF. To this reaction mixture was added dropwise 33 g (2.27 mol) of propionic anhydride at r.t with stirring. After 90 min, the reaction was stopped and reaction mixture washed with 5% aq. NaHCO₃ solution. The organic layer was separated and washed with 1 N HCl and brine. The aqueous layers were extracted with dichloromethane. The organic layers were combined and washed with brine. After being dried over Na₂SO₄, the crude product was obtained from rotary evaporation and dried in vacuo to give 62 g crude product. TLC (1:1 Hexane/EtOAc) analysis indicated that the crude material contained the desired product (7a), starting compound 2 and a small amount of 5,7-diester 8a. The obtained crude product was then purified by silica gel column chromatography on a Biotage column eluting with 2:1 Hexane/EtOAc to give 12 g of 7a (36% yield) as white solid. mp: 166-168° C.; ¹H NMR (DMSO-d₆), δ 0.97 (3H, t, J=7.2 Hz), 1.14 (3H, t, J=7.4 Hz), 1.62 (2H, sextet, J=7.5 Hz), 2.62 (2H, q, J=7.5 Hz), 2.92 (2H, t, J=7.5 Hz), 6.09 (1H, s), 6.57 (1H, d, J=2.4 Hz), 6.67 (1H, d, J=2.4 Hz), 11.10 (1H, s); ¹³C NMR (CDCl₃), δ 8.6, 13.7, 22.4, 26.9, 37.2, 101.4, 105.3, 106.2, 111.9, 153.0, 155.9, 157.2, 158.0, 159.7, 172.2; IR (film): 3300-3075, 2968, 1758, 1676, 1610, 1433, 1126 cm⁻¹; MS m/e 277 (M+1); Anal. Calcd. for C₁₅H₁₆O₅: C, 65.21; H, 5.84. Found: C, 64.61; H, 5.86.

### EXAMPLE 10

5-Hydroxy-7-pivaloyloxy-4-propylcoumarin (7b):

To a solution of coumarin 2 (1.10 g, 5 mmol) in THF (10 mL) was added pyridine (2.02 mL, 25 mmol), followed by pivaloyl chloride (0.612 mL, 5 mmol), and the reaction mixture was allowed to stir at room temperature for 6 days. The pyridinium hydrochloride was removed by filtration and washed a few times with ethyl acetate. The organic solutions were combined and washed, successively, with 1M HCl (2×25 mL), water (25 mL), aqueous saturated sodium bicarbonate (25 mL). After being dried over sodium sulfate and concentrated under vacuum, the crude product was purified by silica gel chromatography (8:1 hexane/ethyl acetate to 2:1 hexane/ethyl acetate) to obtain 8b (350 mg, 18% yield) and 7-pivaloylated coumarin 7b (550 mg, 36% yield) as a white solid. For compound 7b, mp: 158-160° C.; R_{f}=0.32 (4:1 hexane/ethyl acetate); ¹H NMR (CDCl₃), δ 0.97 (3H, t, J=7.8 Hz), 1.37 (9H, s), 1.56 (2H, sextet, J=7.4 Hz), 2.84 (2H, t, J=7.8 Hz), 6.07 (1H, s), 6.43 (1H, d, J=2.4 Hz), 6.61 (1H, d, J=2.1 Hz), 8.09 (1H, s); ¹³C NMR (CDCl₃), δ 13.8, 22.4, 26.9, 37.8, 39.3, 102.4, 105.8, 107.2, 112.1, 153.3, 156.2, 156.3, 159.3, 161.9, 178.0; IR (film): 3358, 2971, 2365, 1730, 1615, 1431, 1275, 1146 cm⁻¹; MS m/e 305 (M+1); Anal. Calcd. for C₁₇H₂₀O₅: C, 67.09; H, 6.62. Found: C, 66.80; H, 6.70.

### EXAMPLE 11

5,7-bis(pivaloyloxy)-4-propylcoumarin (8b):

To a solution of 5,7-dihydroxy-4-propylcoumarin (2) (1.10 g, 5 mmol) in pyridine (12 mL) and THF (6 mL) was added pivaloyl chloride (0.673 mL, 5.5 mmol) and the reaction mixture was allowed to stir at room temperature for 24 h. TLC revealed formation of a new less polar spot and unreacted starting material. In an effort to drive the reaction to completion, pivaloyl chloride (0.50 mL) was added and the reaction continued to stir at room temperature for another 72 h. The pyridinium hydrochloride was removed by filtration and washed a few times with ethyl acetate. The organic solutions were combined and washed, successively, with 1 M HCl (2×25 mL), water (25 mL), aqueous saturated sodium bicarbonate (25 mL). After being dried over sodium sulfate and concentrated under vacuum, the crude product was purified by silica gel chromatography (2:1 hexane/ethyl acetate) to obtain 8b as a white solid (1.90 g, 98% yield). mp 110-112° C.; R_{f}=0.49 (4:1 hexane/ethyl acetate); ¹H-NMR (CDCl₃), δ: 1.03 (3H, t, J=7.8 Hz), 1.36 (9H, s), 1.41 (9H, s), 1.69 (2H, sextet, J=7.4 Hz), 2.81 (2H, t, J=7.8 Hz), 6.22 (1H, s), 6.60 (1H, d, J=2.4 Hz), 7.03 (1H, d, J=2.1 Hz); ¹³C-NMR (CDCl₃) δ: 13.5, 20.7, 26.4, 26.8, 26.9, 36.4, 39.2, 39.4, 108.6, 111.5, 113.4, 114.2, 149.4, 152.7, 154.9, 155.4, 159.9, 177.1; IR (film): 3090, 2971, 2941, 2876, 1757, 1615, 1481, 1422, 1273 cm⁻¹; MS m/e 389 (M+1); Anal. Calcd. for C₂₂H₂₈O₆: C, 68.02; H, 7.26. Found: C, 67.77; H, 7.18.

### EXAMPLE 12

7-TBDMS and 5,7-bis(TBDMS) Substituted Coumarin (7c and 8c):

A mixture of coumarin 2 (5.0 g, 23 mmol), TBDMS-Cl (5.8 g, 27 mmol), and imidazole (4.7 g, 69 mmol) in 50 ml of dry DMF was stirred at room temperature under nitrogen for 20 h, whereupon EtOAc (300 ml) was added to the reaction mixture. The precipitates formed were removed by filtration. The filtrate was washed, successively, with 1N HCl (100 mL×2), water (100 mL×3), and brine (200 mL). The organic layer was then dried with Na₂SO₄. After removal of the drying agent by filtration, the organic solution was kept at room temperature and crystals were formed. The solid was collected. The mother liquor was concentrated and the residue was recrystallized in EtOAc. This process was repeated two more times to give overall 2.2 g (28% yield) of 7c as white crystals. The residue from the mother liquor was further purified by column chromatography to give 2.5 g (24% yield) of solid that was assigned the structure of bis-TBDMS ether 8c, additional 0.2 g of 7c (combined yield of 31%), and 0.2 g of unreacted starting material 2. The analytical data of 7c were: mp 220-223° C.; ¹H NMR (acetone-d₆) δ 0.28 (6H, s), 1.00 (12H, m), 1.69 (2H, m), 2.95 (2H, t, J=7.5 Hz), 5.91 (1H, s), 6.33 (1H, d, J=2.7 Hz), 6.42 (1H, d, J=2.4 Hz), 9.55 (1H, s); ¹³C NMR (DMSO-d₆) 6-4.7, −3.9, 13.7, 22.4, 25.4, 37.1, 99.2, 103.3, 103.5, 109.8, 156.7, 157.5, 158.4, 158.5, 160.1; IR 3497-3021 (s, broad), 1684 (s, sharp), 1616 (s, sharp) cm⁻¹; LRMS m/e: 335 (M+1); Anal. Calcd. for C₁₈H₂₆O₄Si: C, 64.64; H, 7.83. Found: C, 64.31; H, 7.78. For 8c, mp 78-79° C.; ¹H NMR δ 0.24 (6H, s), 0.36 (6H, s), 0.95-9.97 (21H, m), 1.59 (2H, q, J=7.5 Hz), 2.91 (2H, t, J=7.5 Hz), 6.02 (1H, s), 6.21 (1H, d, J=2.7 Hz), 6.48 (1H, d, J=2.4 Hz).

### EXAMPLE 13

5,7-Bis(tosyloxy)-4-propylcoumarin (8d):

A mixture of coumarin 2 (30 g, 0.14 mol), potassium carbonate (76 g, 0.55 mol), p-toluenesulfonyl chloride (57 g, 0.3 mol) and acetone (450 ml) was refluxed for 4.5 h. After cooling, the mixture was filtered and the filtrate evaporated to give a light-yellow solid. The solid residue was dissolved in 1.2 L of EtOAc and 1 L of water. The aqueous layer was removed and back-extracted with EtOAc (2×200 ml). The combined organic layers was washed with brine, dried over Na₂SO₄, filtered, and concentrated under vacuum to afford 75 g of crude product as a light-yellow solid. The crude material was triturated with EtOAc (to remove the bottom spot on TLC which was more polar than the desired product), then filtered to give a white solid and an orange filtrate. The white solid was triturated with hexane (to remove the top spot on TLC which was less polar than the desired product), then filtered to give 51.8 g of product as a white powder. The orange filtrate was concentrated to afford 22 g of residue as a dark-orange oil which was solidify by adding hexane. The solid was collected by filtration, triturated with EtOAc, filtered, and washed with hexane to give an additional 11.7 g of product as a white powder. The overall yield was 63.5 g (88%): mp 110-112° C.; ¹H NMR (d₆-DMSO) δ 0.82 (1H, t, J=7.2 Hz), 1.44 (2H, m), 2.44 (3H, s), 2.46 (3H, s), 2.72 (1H, t, J=7.6 Hz), 6.36 (1H, s), 6.80 (1H, d, J=2.4 Hz), 7.20 (1H, d, J=2.4 Hz), 7.50-7.55 (4H, m), 7.76-7.82 (4H, m); ¹³C NMR (d₆-DMSO) δ 13.3, 21.2, 21.5, 36.1, 110.1, 111.7, 112.1, 116.3, 128.4, 128.5, 130.7, 130.8, 130.9, 146.3, 146.8, 147.2, 149.8, 154.0, 155.1, 158.2; IR 1740, 1615 cm⁻¹; LRMS calcd for C₂₆H₂₀O₈S₂ 528.6, found 529.1. Anal. Calcd for C₂₆H₂₀O₈S₂: C, 59.08; H, 4.58. Found: C, 58.97; H, 4.58.

### EXAMPLE 14

5-Hydroxy-4-propyl-7-tosyloxy-coumarin (7d):

To a 1L three-necked round-bottomed flask equipped with a mechanical stirrer, an additional funnel, a thermometer, and N₂ inlet/outlet were added 60 g (0.113 mol) of 8d and 300 ml of THF. The solution was cooled to 0° C., and 125 ml (0.125 mol) of a 1.0 M solution of tetrabutylammonium fluoride in THF was added. The resulting mixture was stirred at 0° C. for 5 hours. The solvent was removed to give a green-brown oil which was diluted with 1 L of EtOAc, washed with water (500 ml). The aqueous layer was extracted with EtOAc (2×250 ml). The organic layers were combined, washed with brine (300 ml), dried over Na₂SO₄, and filtered. The solvent was removed under vacuum to provide 100 g of crude product as a thick green-brown oil. The crude was purified by filtering through a column of silica gel with EtOAc first and the solid obtained was recrystallized from EtOAc afford 24 g (57%) of the desired product as a white solid: mp 214-215° C.; ¹H NMR (d₆-DMSO) δ 0.94 (3H, t, J=7.2 Hz), 1.57 (2H, m), 2.44 (3H, s), 2.88 (2H, t, J=7.5 Hz), 6.11 (1H, s), 6.49 (1H, d, J=2.4 Hz), 6.55 (1H, d, J=2.4 Hz), 7.51 (2H, d, J=8.1 Hz), 7.82 (2H, d, J=8.1 Hz), 11.29 (1H, s); ¹³C NMR (d₆-DMSO) δ 13.7, 21.2, 22.3, 37.0, 101.3, 105.0, 107.4, 112.6, 128.4, 130.5, 131.4, 146.3, 150.9, 155.7, 157.5, 157.6, 159.3. Anal. Calcd for C₁₉H₁₈O₆S: C, 60.95; H, 4.85. Found: C, 60.85; H, 4.83.

### EXAMPLE 15

2,2-Dimethyl-5-propionyloxy-10-propyl-2H,8H-benzo[1,2-b:3,4-b′]dipyran-8-one (5a):

To a solution of 7-propionate 7a (0.83 g, 3.0 mmol) in 2-butanone (40 mL) and DMF (4 mL) were added tetrabutylammonium iodide (1.11 g, 3 mmol), K₂CO₃ (1.04 g, 7.5 mmol), and 3-chloro-3-methyl-1-butyne (1.11 g, 3 mmol). The reaction mixture was heated at 60° C. for 1 h before ZnCl₂ (3.9 mL of 1.0 M solution in ether, 3.9 mmol) was added. The temperature was then raised to 70° C. and maintained at that temperature for 21 h. The reaction mixture was cooled to room temperature and quenched with saturated aqueous NH₄Cl (100 mL). The mixture was extracted with EtOAc (100 mL×2) and the combined organic layers were washed with brine (100 mL) and dried over Na₂SO₄. Evaporation of the solvent gave the crude product (1.9 g). After column chromtographic purification, 280 mg (27.1% yield) of the desired product 5a was obtained as a waxy solid. ¹H NMR (DMSO-d₆) δ 1.00 (3H, t, J=7.2 Hz), 1.16 (3H, t, J=7.5 Hz); 1.47 (6H, s), 1.61 (2H, m), 2.71 (2H, q, J=7.5 Hz), 2.89 (2H, t, J=7.8), 5.84 (11H, d, J=9.9 Hz), 6.17 (11H, s), 6.41 (1H, d, J=10.2 Hz); ¹³C NMR (DMSO-d₆) δ 8.6, 13.7, 22.8, 26.6, 27.2, 37.4, 78.3, 103.6, 107.2, 110.8, 113.4, 115.5, 130.1, 148.5, 151.4, 154.4, 156.9, 159.3, 172.2; IR 1767 (s and sharp), 1723 (s and sharp), 1616 (s and sharp) cm⁻¹; MS m/e 343 (M+1); Anal. Calcd. for C₂₀H₂₂O₅: C, 70.16; H, 6.47. Found: C, 70.37; H, 6.51.

### EXAMPLE 16

2,2-Dimethyl-5-pivaloyloxy-10-propyl-2H,8H-benzo[1,2-b:3,4-b′]dipyran-8-one (5b):

To a suspension of compound 7b (304 mg, 1 mmol) in 2-butanone (13 mL) and DMF (1.3 mL) was added potassium carbonate (346 mg, 2.5 mmol), 3-chloro-3-methyl-1-butyne (0.56 mL, 5 mmol) and tetrabutylammonium iodide (360 mg, 1 mmol). The reaction mixture was heated to 60° C. for 1 h, then anhydrous ZnCl₂ (1.0 M solution in ether, 1.3 mL) was added. The reaction mixture was heated to 70° C. for 26 h, then cooled to r.t., quenched with saturated aqueous NH₄Cl (25 mL), and extracted with ethyl acetate (2×75 mL). The organic solutions were combined, washed with brine, dried (Na₂SO₄), and concentrated. The crude product obtained was purified by silica gel chromatography (8:1 hexane/ethyl acetate) to obtain 5b as a yellow solid (270 mg, 73%). mp: 65-68° C.; R_{f}: 0.54 (4:1 hexane/ethyl acetate); ¹H NMR (CDCl₃) δ 1.04 (3H, d, J=7.2 Hz), 1.39 (9H, s), 1.52 (6H, s), 1.66-1.71 (2H, m), 2.90 (2H, t, J=7.8 Hz), 5.62 (1H, d, J=10.2 Hz), 6.07 (1H, s), 6.30 (1H, d, J=10.2 Hz), 6.62 (1H, s); ¹³C NMR (CDCl₃) δ 13.8, 22.9, 26.9, 27.8, 38.3, 39.3, 78.1, 103.5, 107.9, 110.9, 113.4, 115.9, 129.1, 148.9, 152.0, 155.1, 157.4, 160.6, 176.2; IR (film): 2967, 1750, 1616, 1364, 1142 cm⁻¹; MS m/c 371 (M+1); Anal. Calcd. for C₂₂H₂₆O₅: C, 71.33; H, 7.07. Found: C, 71.08; H, 7.35.

### EXAMPLE 17

2,2-Dimethyl-5-tosyloxy-10-propyl-2H,8H-benzo[1,2-b:3,4-b′]dipyran-8-one (5d):

To a 100 mL three-necked round-bottomed flask equipped with a mechanical stirrer, a condenser, a thermometer, and N₂ inlet/outlet were added 0.5 g (1.34 mmol) of 7d, 20 ml of 2-butanone, and 2 ml of DMF. This was followed by the addition of 0.46 g (3.34 mmol) of K₂CO₃, 0.49 g (1.33 mmol) of Bu₄NI. To this reaction mixture, 0.44 ml (4.0 mmol) of 3-chloro-3-methyl-1-butyne was added by syringe. The solution was heated to 60° C. for 1 h, and 1.74 ml of 1 M solution of ZnCl₂ in ether was added. The reaction mixture was then heated to 70° C. and stirred for 40 h at that temperature. After cooled to room temperature, the mixture was diluted with EtOAc (100 ml), and quenched with saturated aqueous NH₄Cl. The aqueous layer was extracted with EtOAc (2×50 ml). The combined EtOAc solution was washed with brine, dried over Na₂SO₄, and filtered. The solvent was removed under vacuum to provide 0.8 g of crude product as a yellow-white solid. The product was purified by column chromatography and the solid obtained were recrystallized from EtOAc to afford 0.3 g (50% yield) of the desired product 5d as a white solid: mp 150-151° C. ¹H NMR (d₆-DMSO) δ 0.97 (3H, t, J=7.1 Hz), 1.34 (6H, s), 1.57 (2H, m), 2.42 (3H, s), 2.84 (2H, t, J=7.6 Hz), 5.67 (1H, d, J=10.2 Hz), 6.20 (2H, m), 6.68 (1H, s), 7.47 (2H, d, J=7.5 Hz), 7.79 (2H, d, J=7.5 Hz); ¹³C NMR (d₆-DMSO) δ 13.6, 21.1, 22.7, 27.0, 37.3, 78.5, 103.4, 108.4, 111.5, 114.1, 114.8, 128.6, 130.3, 130.6, 103.9, 146.1, 146.6, 151.6, 154.2, 156.6, 159.0. Anal. Calcd for C₂₄H₂₄O₆S: C, 65.44; H, 5.49. Found: C, 65.32; H, 5.50.

### EXAMPLE 18

2,2-Dimethyl-5-hydroxy-10-propyl-2H,8H-benzo[1,2-b:3,4-b′] dipyran-8-one (6):

To a solution of ester 5a (223 mg, 0.65 mmol) in 15 mL of MeOH were added saturated aqueous solution of NaHCO₃ (7 mL) and water (7 mL). The reaction mixture was stirred at room temperature under nitrogen for 7 h until TLC indicated complete consumption of the starting material. The reaction mixture was then acidified with 10% aqueous HCl (100 mL), and extracted with EtOAc (50 mL). The organic solution was washed with brine (100 mL) and dried with Na₂SO₄. Evaporation of the solvent yielded the crude product that was purified by preparative TLC to afford 97 mg (52% yield) of 6 as a solid. mp 190-192° C.; ¹H NMR (DMSO-d₆) δ 0.99 (3H, t, J=7.3 Hz), 1.45 (6H, s), 1.59 (2H, m), 2.83 (2H, t, J=7.6 Hz), 5.66 (1H, d, J=9.9 Hz), 5.92 (1H, s), 6.34 (1H, s), 6.57 (1H, d, J=9.9 Hz), 10.77 (1H, s); ¹³C NMR (DMSO-d₆) δ 13.7, 22.9, 27.2, 37.5, 77.5, 95.7, 102.2, 106.1, 109.9, 116.3, 127.1, 151.7, 155.7, 156.4, 157.8, 160.0, 188.9; IR (film): 3185, 1686, 1582, 1381, 1157 cm⁻¹; MS m/e: 287 (M+1); Anal. Calcd. for C₁₇H₁₈O₄: C, 71.31; H. 6.34. Found: C, 71.39; H, 6.40.

### EXAMPLE 19

Synthesis of Methyl 3-Hydroxy-2-methylbutyrate 11 (X=OH, Y=OMe) from D-Threonine According to the Literature Method:⁵²

An aqueous solution of D-threonine (5.95 g in 50 ml water) was treated with 48% aqueous HBr (10 ml) and KBr (21.0 g). The mixture was cooled to −15° C. and NaNO₂ (3.8 g) was slowly added to in small portions over 2.5 h. After overnight stirring while the temperature was warmed up to room temperature (3×150 mL), the mixture was extracted with ether. The ether solution was dried (Na₂SO₄) and concentrated in vacuo to give the crude bromoacid as an oil. The bomoacid was dissolved in absolute EtOH (75 mL) and cooled to −30° C. A solution of KOH (5.05 g) in ethanol (40 mL) was added slowly, and the reaction mixture was stirred overnight while the temperature was allowed to warm up to room temperature. The solid KBr was removed by filtration and the filtrate was concentrated under reduced pressure. The residue was transferred to a solution of 18-crown-6 (10.55 g) in methylene chloride (100 mL). Dimethyl sulfate (3.56 g) was added to the reaction mixture and stirring was continued for 2 h. Ether was added and the precipitate formed was filtered off. The volatile solvent was removed by simple distillation to give crude epoxide that still contained the crown ether (1.3 g): 1H NMR (CDCl₃) δ 1.39 (3H, d, J=5.1 Hz), 3.30 (1H, m), 3.52 (1H, d, J=4.5 Hz), 3.81 (3H, s).

A solution of MeLi in ether (21.7 mL of 1.4 M solution) was added to a stirred suspension of CuI (3.07 g) in ether (30 mL) at −30° C. After 15 min of stirring, a solution of the crude epoxide (1.3 g) in ether (24 mL) was added to the reaction mixture. The mixture was stirred for 2 h before a solution of concentrated ammonium hydroxide in saturated ammonium chloride was added. The resulting biphasic solution was extracted with EtOAc and the combined organic solution was dried and the solvent was removed by simple distillation to give a complex mixture (0.26 g) that might contain 11 (X=OH, Y=OMe) as a yellow oil.

### EXAMPLE 20

Synthesis of 3-Hydroxy-2-methylbutyric Acid 11 (X=OH, Y=OH) from Ethyl 2-methylacetoacetate According to the Literature Method:^{53,54}

To a 6 L Erlenmeyer flask containing Baker's yeast (10 g) and sucrose (150 g) was added tap water (1000 mL) and the reaction mixture was mechanically stirred at room temperature for 0.5 h. Ethyl 2-methylacetoacetate (10 g, 0.0694 mol) was added to the reaction mixture and stirring continued for 24 h. Sucrose (50 g) was added and stirring continued for another 24 h. Hyflo super cel (50 g) was added to the reaction mixture and filtered through a sintered glass funnel. The aqueous solution was then extracted with ether (1.5 L), dried over sodium sulfate, and filtered. The solvent ether was removed via simple distillation to obtain ethyl 3-hydroxy-2-methylbutyrate (10 g, 100%) as a mixture of two isomers: ¹H NMR (CDCl₃) δ 1.18-1.30 (18H, m), 2.47-2.52 (2H, m), 3.85-3.91 (1H, m), 4.05-4.10 (1H, m), 4.13-4.21 (4H, m).

To ethyl 2-methyl-3-hydroxybutyrate obtained above (10 g, 0.0694 mol) was added 30% aqueous sodium hydroxide (40 mL), and the mixture was stirred at room temperature for 4 h. The reaction mixture was concentrated under vacuum to obtain a yellow solid. The solid was dissolved in 1M HCl (100 mL) and extracted with ether (3×100 mL), dried over sodium sulfate and concentrated under vacuum to obtain 3-hydroxy-2-methyl butyric acid (2.0 g, 24%) as a mixture of diastereomers. For the major isomer: ¹H NMR (CDCl₃) δ 1.21-1.24 (6H, m), 2.57-2.62 (1H, m), 4.11-4.15 (1H, m).

### EXAMPLE 21

Synthesis of Oxazolidinone 11 (X=OH, Y=Oxazolidinone) According to the Literature Method.⁵⁵

A 7 ml aliquot of freshly prepared LDA (0.5 M in hexane-ether) was cooled at −78° C., and to this solution was added dropwise Evan's oxazolidinone in 20 ml ether. The reaction was stirred for 30 min, followed by the dropwise addition of chlorotitanium triisopropoxide (9 ml 1.0M in hexane, 3 mmol) at −78° C. The solution was allowed to warm up to −40° C. over 1 h then cooled down to −78° C. Acetaldehyde was added in one portion via a cold syringe. The temperature was maintained between −78° C.˜−40° C. under nitrogen for 3 h. Saturated aqueous solution of NH₄Cl (5 mL) was added. After filtration and extraction, the crude product (690 mg) was purified by column chromatography to afford 429 mg (62.4%) product 11 (X=OH, Y=Oxazolidinone) as an oil: [α]_{D}=+154.0° (c 0.5, MeOH); ¹H NMR (CDCl₃) δ 0.91 (3H, d, J=6.6 Hz), 0.93 (3H, d, J=7.2 Hz), 1.17 (3H, d, J=6.9 Hz), 1.22 (3H, d, J=6.6 Hz), 2.38 (1H, m), 3.90 (1H, dq J=3,6, 6.5 Hz), 4.16 (1H, ddd, J=3.3, 6.4, 12.9 Hz), 4.23 (1H, dd, J=3.2, 9.0 Hz), 4.29 (1H, apparent t, J=8.6 Hz), 4.47 (1H, m); ¹³C NMR (CDCl₃) δ 10.5, 14.6, 17.8, 19.2, 28.5, 42.8, 58.6, 63.3, 68.5, 154.3, 176.6; IR 3302-3650 (m, broad), 1780 (s, sharp), 1699(s, sharp) cm⁻¹; LRMS cacld for C₁₁H₁₉NO₄ 229.3, found 229.9. Anal. Calcd for C₁₁H₁₉NO₄: C, 57.6; H, 8.4; N, 6.1. Found: C, 57.78; H 8.38; N, 6.07.

### EXAMPLE 22

Synthesis of Oxazolidinone 11 (X=OTs, Y=Oxazolidinone):

Alcohol 11 (X=OH, Y=Oxazolidinone) (242 mg, 1.06 mmol) was dissolved in 2 mL pyridine, and the solution was stirred at −20° C. while TsCl (262 mg, 1.38 mmol) was added quickly under nitrogen. The temperature was allowed to rise to room temperature. Stirring was continued for 42 h. Water (5 ml) was added slowly at −20° C. then the reaction was stirred for 40 min and diluted with 10 ml EtOAc. The two layers were separated and the organic layer was washed with 1N HCl (10 mL×3), brine (10 mL), and dried with sodium sulfate. Evaporation of the solvent in vacuo afforded crude tosylate 11 (X=OTs, Y=Oxazolidinone) (192 mg). After preparative TLC purification, 92 mg (23%) of product was obtained as an oil: ¹H NMR (CDCl₃) δ ppm, 0.90 (6H, t, J=6.6 Hz), 1.13 (3H, d, J=7.2 Hz), 1.30 (3H, d, J=6.6 Hz), 2.33 (1H, m), 2.44 (1H, m), 3.98 (1H, m), 4.21-4.29 (2H, m), 4.44 (1H, m), 5.00 (1H, m); ¹³C NMR δ 12.9, 14.4, 17.9, 19.1, 21.5, 28.2, 42.9, 58.6, 63.2, 79.6, 127.9, 129.8, 134.4, 144.7, 153.9, 173.2. Anal. Calcd. for C₁₈H₂₅O₆SN: C, 56.38; H, 6.57; N, 3.65; Found: C, 56.12; H, 6.64; N, 3.51.

### EXAMPLE 23

Synthesis of 1,3-Dihydroxy-2-methylbutane 12 (Z=H):

To ethyl 3-hydroxy-2-methylbutyrate (7.3 g, 50 mmol) in ether (250 mL) at 0° C., added LiAlH₄ (5.9 g, 155.5 mmol). The gray solution was stirred at 0° C. for 10 minutes, cooling bath removed and stirring continued at room temperature for 6 h. The reaction mixture was cooled to 0° C. and quenched slowly by dropwise addition of water (6 mL), 1M NaOH (6 mL), water (6 mL). Excess MgSO₄ (100 g) was added to the reaction mixture and allowed to stir at room temperature overnight. Filtered the reaction mixture and washed the solid with ether (400 mL). The ether extracts was concentrated under vacuum to obtain 1,3-dihydroxy-2-methylbutane 12 (Z=H) (2.6 g, 50%) as a yellow oil. ¹H-NMR (CDCl₃) δ 0.89 (3H, d, J=6.9 Hz), 1.19 (3H, d, J=6.6 Hz), 1.81 (1H, m), 3.70 (2H, m), 4.04 (1H, m); MS [M−1]⁺103.5.

### EXAMPLE 24

Synthesis of the Protected 1,3-Dihydroxy-2-methylbutane 12 (Z=TBDMS):

To a suspension of sodium hydride (424 mg, 10.6 mmol) in THF (30 mL) added 12 (Z=H) (1.1 g, 10.6 mmol) and stirred at room temperature for 45 min, at which time a large amount of an opaque white precipitate had formed. The tert-butyldimethylsilyl chloride (1.59 g, 10.6 mmol) was added and the reaction mixture was allowed to stir at room temperature for 1.5 h. The reaction mixture was diluted with ether (300 mL) and washed with 10% aqueous potassium carbonate (90 mL), brine (75 mL). The organic extracts were dried over sodium sulfate, concentrated under vacuum and purified by silica gel chromatography (8/1 hexane/ethyl acetate) to obtain 12 (Z=TBDMS) (2.3 g, 99%) as a colorless oil. ¹H NMR (CDCl₃) δ 0.08 (6H, s), 0.90 (12H, s,d), 1.16 (3H, d, J=6.3 Hz), 3.72 (2H, m), 3.99 (1H, m); ¹³C NMR (CDCl₃) 8-5.8 (2C), 10.5, 19.5, 25.7, 25.9, 39.8, 70.7; Anal. Calcd for C₁₁H₂₅O₂Si: C, 60.55; H, 11.93. Found C, 61.01; H, 11.84; IR (film): 3449, 2957, 2859, 1464, 1256 cm⁻¹; MS [M+1]⁺219.1.

### EXAMPLE 25

Compound 14 (Z=TBDMS):

To monophenol 6 (900 mg, 3.14 mmol), triphenylphosphine (1.24 g, 4.73 mmol) and 12 (R=TBDMS) (1.1 g, 5.05 mmol) in THF (60 mL) added DEAD (800 μL, 5.08 mmol) and stirred at room temperature under nitrogen overnight. The reaction mixture was concentrated under vacuum and purified by silica gel chromatography (3/1 hexane/ethyl acetate) to obtain 14 (Z=TBDMS) (1.53 g, 100%) as an oil. ¹H NMR (CDCl₃) δ −0.08 (6H, m), 0.88 (9H, s), 0.96 (3H, d, J=7.2 Hz), 1.03 (3H, t, J=7.2 Hz), 1.27 93H, d, J=6.3 Hz), 1.49 (6H, s), 1.66 (2H, m), 2.08 (1H, m), 2.88 (2H, t, J=7.6 Hz), 3.56 (2H, m), 4.51 (1H, m), 5.52 (1H, d, J=9.9 Hz), 5.94 ((1H, s), 6.42 (1H, s), 6.64 (1H, d, J=9.9 Hz); ¹³C-NMR (CDCl₃) δ −5.7 (2C), 11.9, 14.0, 15.4, 18.1, 23.1, 25.8 (3C), 27.7 (2C), 38.3, 40.3, 64.6, 65.4, 75.2, 94.2, 103.7, 107.8, 110.8, 116.9, 126.7, 151.9, 156.3, 158.2, 161.4; Anal. Calcd for C₂₈H₄₂O₅Si: C, 69.03; H, 8.63. Found C, 69.33; H, 8.84; IR (film): 2928, 2857, 1738, 1605 cm⁻¹; MS [M+1]⁺487.2.

### EXAMPLE 26

Compound 14 (Z=H):

To 14 (Z=TBDMS) (1.5 g, 3.08 mmol) in THF (45 mL) added TBAF (1.0 M soln in THF, 5 mL, 5 mmol) and stirred at room temperature overnight. The reaction mixture was acidified with 1M HCl and extracted with ethyl acetate. The organic extracts were then washed with water, brine, dried (sodium sulfate) and concentrated under vacuum. The crude product was purified by silica gel chromatography (1/1 hexane/ethyl acetate) to obtain 14 (Z=H) (800 mg, 70%) as an oil. ¹H-NMR (CDCl₃) δ 0.87 (3H, d, J=7.8 Hz), 1.06 (3H, t, J=6.9 Hz), 1.32 (3H, d, J=6.3 Hz), 1.49 (6H, s), 1.65 (2H, m), 2.09 (1H, m), 2.88 (2H, t, J=7.6 Hz), 3.68 (2H, d, J=5.7 Hz), 4.50 (1H, m), 5.53 (1H, d, J=9.9 Hz), 5.95 (1H, s), 6.4 (1H, s), 6.62 (1H, d, J=10.2 Hz); ¹³C-NMR (CDCl₃) δ 12.4, 13.9, 16.2, 23.1, 27.7 (2C), 38.3, 40.4, 65.0, 75.0, 94.2, 104.0, 107.8, 110.9, 116.6, 126.9, 151.9, 156.0, 156.5, 158.2, 161.4; Anal. Calcd for C₂₂H₂₈O₅+0.7 eq H₂O: C, 68.62; H, 7.70. Found C, 68.71; H, 7.85; IR (film): 3464, 2969, 2874, 1738, 1593 cm⁻¹; MS [M+1]⁺373.1.

### EXAMPLE 27

Compound 13 (Y=H):

To CH₂Cl₂ (1 mL) at −78° C. added oxalyl chloride (20 μL, 0.223 mmol), followed by DMSO (33 μL, 0.459 mmol). After 5 minutes added alcohol 14 (R=H) (57 mg, 0.153 mmol) in CH₂Cl₂ (2 mL). The reaction mixture was stirred at −78° C. for 30 minutes. Triethylamine (107 μL, 0.765 mmol) was added to the reaction mixture and allowed to warm to room temperature over 2 h. The reaction mixture was concentrated under vacuum and purified by silica gel chromatography to obtain 13 (Y=H) (46 mg, 82%) as an oil. ¹H-NMR (CDCl₃) δ 1.03 (3H, t, J=7.2 Hz), 1.21.(3H, d, J=6.9 Hz), 1.28 (3H, d, J=6.9 Hz), 1.49 (6H, s), 1.55-1.70 (2H, m), 2.72-2.77 (1H, m), 2.89 (2H, t, J=7.2 Hz), 4.72 (1H, m), 5.52 (1H, d, J=10.2 Hz), 5.97 (1H, s), 6.42 (1H, s), 6.55 (1H, d, J=10.2 Hz), 9.79 (1H, d, J=2.4 Hz); MS [M+1]⁺371.2.

### EXAMPLE 28

Compound 13 (Y=OH):

To aldehyde 13 (Y=H) (43 mg, 0.116 mmol) in acetone (4 mL) added 2-methyl-2-butene (1 mL, 2.0 M solution in THF). To the above reaction mixture added sodium chlorite (100 mg, 1.10 mmol) and sodium dihydrogenphosphate (96 mg, 0.800 mmol) in water (2 mL) and allowed to stir at room temperature overnight. The reaction mixture was diluted with water and extracted with ethyl acetate, dried (Na₂SO₄) and concentrated under vacuum to obtain 13 (Y=OH) (45 mg, 100%) as an oil. ¹H-NMR (CDCl₃) δ 1.04 (3H, t, J=7.2 Hz), 1.22 (3H, d, J=6.9 Hz), 1.27 (3H, d, J=6.9 Hz), 1.49 (6H, s), 1.52-1.67 (2H, m), 2.86-2.93 (3H, m), 4.73 (1H, m), 5.52 (1H, d, J=9.9 Hz), 5.97 (1H, s), 6.45 (1H, s), 6.61 (1H, d, J=9.9 Hz); MS [M+1]⁺387.2.

### EXAMPLE 29

Compound 19 (R₇=R₉=R₁₀=H, R₈=Me, X=OTs):

A solution of methyl acetoacetate (20 g, 0.17 mol) in MeOH (100 ml) was added dropwise to a stirred solution of NaBH₄ (2 g, 0.05 mmol) in MeOH (200 ml) at room temperature. The reaction was monitored by TLC. After 1 hour of stirring, no reaction occurred. An additional 1 g (0.03 mol) of NaBH₄ was added into the reaction mixture, and another portion of NaBH₄ (1 g, 0.03 mol) after 0.5 h of stirring. Stirring was continued for 0.5 hour, and no starting compound was detected by TLC. The MeOH was removed to afford a residue as a clear oil. The residue was washed with 1N HCl (150 ml), then extracted into EtOAc (3×200 ml). The combined organic layers were washed with brine, dried over Na₂SO₄, and concentrated to afford 13 g of crude product methyl 3-hydroxybutyrate as a light-yellow oil which was used for the following tosylation without further purification.

To a solution of methyl 3-hydroxybutyrate (13 g, 110 mmol) in anhydrous pyridine (100 ml) was added p-toluenesulfonyl chloride (31.5 g, 165 mmol) at 0° C. under N₂. After two days of stirring at 0° C., the reaction mixture was cooled to −5° C. followed by the dropwise addition of water (100 ml) while maintaining the temperature <0° C. After an additional 10 min of stirring, more water (200 ml) was added slowly. Crystallization occurred immediately. The temperature was maintained at 0° C. for 1 h, and the crystalline product was filtered, washed with water (5×100 ml), and dried to give 20.6 g (70%) of 19 (R₇=R₉=R₁₀=H, R₈=Me, X=OTs) as a white solid: mp. 45-47° C.; ¹H NMR (d₆-DMSO) δ 1.27 (3H, d, J=6.6 Hz), 2.43 (3H, s), 2.61 (1H, dd, J=15.0, 7.5 Hz), 2.69 (1H, dd, J=15.0, 5.4 Hz), 3.48 (3H, s), 4.86 (1H, m), 7.48 (2H, d, J=8.1 Hz), 7.78 (2H, d, J=8.1 Hz); ¹³C NMR (CDCl₃) δ 20.4, 21.1, 40.5, 51.6, 76.8, 127.6, 130.3, 133.6, 145.0, 169.7; IR 1750 cm⁻¹. Anal. Calcd for C₁₂H₁₆O₅S: C, 52.94; H, 5.88. Found: C 53.10; H 5.95.

### EXAMPLE 30

Compound 10 (R=Me) or 20 (R₁=n-propyl, R₂=R₅=R₆=R₇=R₈=H, R₃=R₄=R₉=R₁₀=Me, X=O, Y=OMe):

To monophenol 6 (430 mg, 1.50 mmol), triphenylphosphine (590 mg, 2.25 mmol) and 19 (R₇=R₈=H, R₉=R₁₀=Me, X=OH) (396 mg, 3 mmol) in dioxane (34 mL) added DEAD (360 μL, 2.25 mmol) and stirred at reflux under nitrogen for 2 h. The reaction mixture was cooled and concentrated under vacuum. The residue was dissolved in ethyl acetate (100 mL) and washed with water (80 mL), dried (sodium sulfate), concentrated under vacuum and purified by silica gel chromatography (3/1 hexane/ethyl acetate) to obtain the corresponding methyl ester of 10 (R=Me) or 20 (R₁=n-propyl, R₂=R₅=R₆=R₇=R₈=H, R₃=R₄=R₉=R₁₀=Me, X=O, Y=OMe) (445 mg, 74%) as a white solid. Mp: 100-101° C.; ¹H NMR (DMSO-d₆) δ 0.99 (3H, t, J=7.5 Hz), 1.26 (6H, s), 1.45 (6H, s), 2.39 (2H, m), 2.86 (2H, t, J=7.5 Hz), 3.63 (3H, s), 4.09 (2H, s), 5.73 (1H, d, J=10.2 Hz), 6.02 (1H, s), 6.45 (1H, d, J=9.6 Hz), 6.63 (1H, s); ¹³C-NMR (CDCl₃) δ 13.8, 22.3, 23.0, 27.7, 38.3, 43.2, 52.1, 74.8, 77.7, 93.3, 104.3, 107.2, 111.2, 116.3, 127.1, 151.7, 156.4, 156.7, 158.1, 161.3, 176.2; MS [M+1]⁺401.1; Anal. Calcd for C₂₃H₂₈O₆: C, 69.0; H, 7.0. Found C, 69.16; H, 7.12; IR (film): 1726, 1605 cm⁻¹.

### EXAMPLE 31

Compound 10 (R=H) or 20 (R₁=n-propyl, R₂=R₅=R₆=R₇=R₈=H, R₃=R₄=R₉=R₁₀=Me, X=O, Y=OH):

To the methyl ester of 10 (R=Me) obtained above (40 mg, 0.1 mmol) in methanol (2 mL) added KOH (25 mg, 0.45 mmol) in water (1 mL) and stirred at room temperature for 3 h. The reaction mixture was concentrated under vacuum. Water was added to the residue, acidified with 1M HCl and extracted with ethyl acetate. The organic extracts were washed with brine, dried (sodium sulfate) and concentrated under vacuum to obtain 10 (R=H) or 20 (R₁=n-propyl, R₂=R₅=R₆=R₇=R₈=H, R₃=R₄=R₉=R₀=Me, X=O, Y=OH) (30 mg, 78%) as a white solid. Mp: 140-142° C. ¹H NMR (DMSO-d₆) δ 0.99 (3H, t, J=7.6 Hz), 1.23 (6H, s), 1.45 (6H, s), 1.59 (2H, m), 2.86 (2H, t, J=8.0 Hz), 4.06 (2H, s), 5.72 (1H, d, J=9.9 Hz), 6.02 (1H, s), 6.50 (1H, d, J=10.2 Hz), 6.63 (1H, s); ¹³C-NMR (CDCl₃) δ 13.8, 22.1, 23.0, 27.7, 38.3, 43.1, 74.4, 77.7, 93.3, 104.3, 107.3, 111.2, 116.4, 127.1, 151.8, 156.4, 156.6, 158.2, 161.4, 181.4; MS [M+1]⁺387.2; Anal. Calcd for C₂₂H₂₆O₆+0.2 eq H₂O: C, 67.75; H, 6.92. Found C, 67.7; H, 6.8; IR (film): 3381, 2750, 1740, 1605 cm⁻¹.

### EXAMPLE 32

Compound 23 (R₇=R₉=R₁₀=H, R₈=Me, Z=TBDMS):

To a suspension of sodium hydride (4 g, 0.1 mol) in THF (200 mL) added 1,3-dihydroxybutane (9 mL, 0.1 mol) and stirred at room temperature for 45 min, at which time a large amount of an opaque white precipitate had formed. The tert-butyldimethylsilyl chloride (15.1 g, 0.1 mol) was added and the reaction mixture was allowed to stir at room temperature overnight. The mixture was diluted with ether (500 mL) and washed with 10% aqueous potassium carbonate (150 mL), brine (100 mL). The organic extracts were dried over sodium sulfate and concentrated under vacuum to obtain a colorless oil (20 g). A portion of the crude product (1 g) was purified by silica gel chromatography (8/1 hexane/ethyl acetate) to obtain 23 (R₇=R₉=R₁₀=H, R₈=Me, Z=TBDMS) (700 mg) as a colorless oil. ¹H NMR (CDCl₃) δ 0.08 (6H, s), 0.90 (9H, s), 1.19 (3H, d, J=6.3 Hz), 1.62-1.70 (2H, m), 3.23 (1H, br s), 3.77-3.99 (2H, m), 4.02-4.07 (1H, m); ¹³C NMR (CDCl₃) 6-5.7 (2C), 23.2, 25.6, 25.7 (3C), 39.8, 62.7, 68.2; MS [M+1]⁺204.9.

### EXAMPLE 33

Compound 14 (R=Me, Z=TBDMS) or 20 (R₁=n-propyl, R₂=R₅=R₆=R₇=R₉=R₁₀=H, R₃=R₄=R₈=Me, X=H₂, Y=OTBDMS):

To monophenol 6 (72 mg, 0.25 mmol), triphenylphosphine (98.4 mg, 0.375 mmol) and 23 (R₇=R₉=R₁₀=H, R₈=Me, Z=TBDMS) (77 mg, 0.375 mmol) in THF (5 mL) added DEAD (60 μL, 0.375 mmol) and stirred at room temperature under nitrogen for 1.5 h. The reaction mixture was concentrated under vacuum and purified by silica gel chromatography (3/1 hexane/ethyl acetate) to obtain 14 (R=Me, Z=TBDMS) or 20 (R₁=n-propyl, R₂=R₅=R₆=R₇=R₉=R₁₀=H, R₃=R₄=R₈=Me, X=H₂, Y=OTBDMS) (111 mg, 94%) as an oil. ¹H NMR (CDCl₃) δ −0.02 (6H, s), 0.89 (9H, s), 1.03 (3H, t, J=7.2 Hz), 1.19 (3H, d, J=6.3 Hz), 1.49 (6H, s), 1.63-1.73 (2H, m), 1.79-2.02 (2H, m), 2.88 (2H, t, J=6.0 Hz), 3.71-3.76 (2H, m), 4.59-4.65 (1H, m), 5.52 (1H, d, J=9.9 Hz), 5.95 (1H, s), 6.44 (1H, s), 6.64 (1H, d, J=9.9 Hz); ¹³C-NMR (CDCl₃) δ −5.6(2C), 13.8, 18.4, 19.6, 23.1, 25.7 (3C), 27.7, 38.3, 39.4, 59.1, 71.7, 94.2, 103.8, 106.3, 107.8, 110.8, 116.9, 126.7, 151.8, 156.3, 156.5, 158.2,161.5; Anal. Calcd for C₂₇H₄₀O₅Si: C, 68.6; H, 8.53. Found C, 68.9; H, 8.6; IR (film): 2959, 2872, 1736, 1605 cm⁻¹; MS [M+1]⁺473.2.

### EXAMPLE 34

Compound 14 (R=Me, Z=H) or 20 (R₁=n-propyl, R₂=R₅=R₆=R₈=R₉=H, R₃=R₄=R₇=Me, X=H₂, Y=OH):

To 20 (R₁=n-propyl, R₂=R₅=R₆=R₇=R₉=R₁₀=H, R₃=R₄=R₈=Me, X=H₂, Y=OTBDMS) (176 mg, 0.372 mmol) in THF (8 mL) added TBAF (1.0 M soln in THF, 560 μL) and stirred at room temperature overnight. The reaction mixture was acidified with 1M HCl and extracted with ethyl acetate. The organic extracts were then washed with water, brine, dried (sodium sulfate) and concentrated under vacuum. The crude product was purified by silica gel chromatography (1/1 hexane/ethyl acetate) to obtain 14 (R=Me, Z=H) or 20 (R₁=n-propyl, R₂=R₅=R₆=R₇=R₉=R₁₀=H, R₃=R₄=R₈=Me, X=H₂, Y=OH) (133 mg, 100%) as an oil. ¹H NMR (CDCl₃) δ 1.03 (3H, t, J=7.5 Hz), 1.38 (3H, d, J=6.0 Hz), 1.49 (6H, s), 1.65-1.79 (2H, m), 1.94-2.05 (2H, m), 2.88 (2H, t, J=6.0 Hz), 3.81 (2H, t, J=5.7 Hz), 4.65-4.67 (1H, m), 5.52 (1H, d, J=9.9 Hz), 5.95 (1H, s), 6.46 (1H, s), 6.62 (1H, J=10.2 Hz); ¹³C-NM(CDCl₃) δ 13.9, 15.1, 19.7, 23.1, 27.7, 38.3, 38.9, 59.3, 72.4, 77.6, 94.2, 104.1, 107.8, 110.9, 116.7, 126.9, 151.9, 156.0, 156.4, 158.2, 161.4; C₂₁H₂₆O₅+0.3 eq H₂O: C, 69.32; H, 7.37. Found C, 69.25; H, 7.39; IR (film): 3474, 2971, 1738, 1593 cm⁻¹; MS [M+1]⁺359.1.

### EXAMPLE 35

In vitro Evaluation of Anti-Viral Agents (Anti-HIV)

This example illustrates the anti-HIV activity of various coumarin and chromene compounds which were evaluated using the published MTT-tetrazolium methods⁸. Retroviral agents AZT and DDC were used as controls for comparison purposes.

The cells used for screening were the MT-2 and the human T4-lymphoblastoid cell line, CEM-SS, and were grown in RPMI 1640 medium supplemented with 10% fetal (v/v) heat-inactivated fetal calf serum and also containing 100 units/mL penicillin, 100 μg/mL streptomycin, 25 mM HEPES and 20 μg/mL gentamicin. The medium used for dilution of drugs and maintenance of cultures during the assay was the same as above. The HTLV-IIIB and HTLV-RF were propagated in CEM-SS. The appropriate amounts of the pure compounds for anti-HIV evaluations were dissolved in DMSO, then diluted in medium to the desired initial concentration. The concentrations (M medium) employed were 0.0032 μM; 0.001 μM; 0.0032 μM; 0.01 μM; 0.032 μM; 0.1 μM; 0.32 μM; 1 μM; 3.2 μM; 10 μM; 32 μM; and 100 μM. Each dilution was added to plates in the amount of 100 μL/well. Drugs were tested in triplicate wells per dilution with infected cells while in duplicate wells per dilution with uninfected cells for evaluation of cytotoxicity. On day 6 (CEM-SS cells) and day 7 (MT-2 cells) post-infection, the viable cells were measured with a tetrazolium salt, MTT (5 mg/mL), added to the test plates. A solution of 20% SDS in 0.001 N HCl is used to dissolve the MTT formazan produced. The optical density value was a function of the amount of formazan produced which was proportional to the number of viable cells. The percent inhibition of CPE per drug concentration was measured as a test over control and expressed in percent (T/C %). The data is summarized in the table below.

Table II below, lists efficacy data for compounds of the present invention against HIV infection.

### EXAMPLE 36

Antiviral Activities of Compounds Against Viruses Other Than HIV

Selected coumarin and chromene compounds, prepared as described above, were evaluated against hepatitis B virus, herpes viruses (HSV-1, HSV-2, HCMV, VZV, and EBV), and respiratory viruses (influenza A, influenza B, parainfluenza, adenovirus, measles, and respiratory syncytial virus). Laboratory procedures for determining antiviral efficacy and toxicity, as well as test design, are described more fully below. Several compounds were found to be active against various viruses and the results are summarized in Table II below.

I. Testing Designed for Determining in vitro Activity and Toxicity of Potential Antiviral Drugs for Herpes Virus Infection

I. Testing Designed for Determining In Vitro Activity And Toxicity
Of Potenial Antiviral Drugs For Herpes virus infection
A. Primary Screening System-Human Foreskin Fibroblast Cells

| | | |
|---|---|---|
| 1. | Antiviral | |
| | HSV-1 or 2 | Semi-automated CPE-inhibition assay |
| | | (HSV-1 E-377 strain; HSV-2 MS strain) |
| | CMV | Semi-automated CPE-inhibition assay |
| | | (AD169 strain) |
| | VZV | Plaque reduction assay |
| | | (Ellen strain) |
| | EBV | Superinfection of Raji or Daudi cells with P3HR-1; |
| | | assay for early antigen (EA) and viral capsid antigen |
| | | (VCA) production |
| 2. | Toxicity | Neutral red uptake-stationary cells |
| | | Cell proliferation assay-rapidly growing cells |

B. Confirmatory Assay Systems-Human Foreskin Fibroblast Cells

| | | |
|---|---|---|
| 1. | Antiviral | |
| | HSV-1 or 2 | Plaque reduction assay-liquid overlay |
| | CMV | Plaque reduction assay-liquid overlay |
| | VZV | Plaque reduction assay or yield reduction |
| | | assay |
| | EBV | P3HR-1 infection of other B-lymphocyte cell |
| | | lines. Inhibition of EBV DNA synthesis |
| | | Hybridization assay |
| 2. | Toxicity | MTT assay for cytotoxicity-stationary cells. |

C. Additional Follow-up Studies

| | | |
|---|---|---|
| 1. | Antiviral | Determine activity in cell lines from other species, i.e. |
| | | mice, rabbits, guinea pigs |
| | | Test sensitivity of other virus strains and |
| | | clinical isolates |
| | | Determine activity against ACV and GCV |
| | | resistant mutants |
| | | Determine mechanism of action |
| 2. | Toxicity | Bone marrow assays-Human CFU-GM and |
| | | BFU-E clonogenic assays |

D. Description of Virus Isolates Used for Antiviral Evaluation

a. Herpes simplex virus type 1 (HSV-I)

1. E-377—laboratory passaged standard strain

2. E-115—laboratory passaged standard strain

3. HL-3—low passaged clinical isolate from herpes labialis

4. HL-34—low passaged clinical isolate from herpes labialis

5. 4E —clinical isolate from herpes encephalitis

6. SC16—ACV sensitive, TK positive

7. SC 16-SI—ACV resistant, TK altered

8. DM 2.1—ACV resistant, TK deficient

9. PAAr—PAA and PFA resistant, polymerase mutant

10. 11893—ACV resistant, TK altered

11. 11359—ACV resistant, TK deficient

12. 11360—ACV resistant, TK deficient

13. B-2006—ACV resistant, TK deficient

b. Herpes simplex virus type 2 (HSV-2)

1. MS—laboratory passaged standard strain

2. X-79—laboratory passaged standard strain

3. Jensen—low passaged clinical isolate from herpes genitalis

4. Heeter—low passaged clinical isolates from herpes genitalis

5. SR—recent clinical isolate from neonatal herpes

6. 8705—ACV sensitive, TK positive

7. 8707—ACV resistant, TK altered

8. 11680—ACV resistant, TK altered

9. 12247—ACV resistant, TK altered

10. 11575—ACV-resistant, TK partial (low producers)

11. 11572—ACV resistant, TK partial (low producers)

12. 11785—ACV resistant, TK partial (low producers)

13. 8711—ACV resistant, TK deficient

14. 11361—ACV resistant, TK deficient

15. AG-3—ACV resistant, TK deficient

c. Human cytomegalovirus (HCMV)

1. AD109—standard laboratory strain

2. Davis—standard laboratory strain

3. Towne—standard laboratory strain

4. EC—recent low passaged clinical isolate

5. LA—recent low passaged clinical isolate

6. CH—recent low passaged clinical isolate

7. Mann—recent low passaged clinical isolate

8. Coffman—recent low passaged clinical isolate

9. C8708/17-1-1—clinical isolate

10. C9207 3-3-1—ganciclovir sensitive

11. C8704 9-4-1—ganciclovir resistant

12. C9208 5-4-2—ganciclovir sensitive

13. C9209 1-4-4—ganciclovir resistant

14. C8912-3—ganciclovir sensitive

15. C8914-6—ganciclovir resistant

16. C8805 37-1-1—ganciclovir resistant

17. C8706 13-1-1—ganciclovir resistant

18. AD169 177^{R}—ganciclovir resistant and HPMPC resistant

d. Murine Cytomegalovirus (MCMV)

1. Smith strain—standard laboratory strain

2. JS strain

e. Varicella Zoster Virus (VZV)

1. Ellen—standard laboratory strain

2. Oka—varicella vaccine strain

3. GLM—recent clinical isolate

4. DKG—recent clinical isolate

5. KS 1027—recent clinical isolate

6. V8907—clinical isolate

7. V8908—Acyclovir resistant mutant of V8907

8. V8602 5-1-1—clinical isolate

9. V8602 7-1-3—ACV resistant, TK deficient

10. V8602 24-3-1—ACV resistant, polymerase mutant

11. 40 A2—ACV resistant

f. Epstein-Barr Virus (EBV)

1. P3HR-1—standard laboratory strain

E. Laboratory Procedures for Determining Antiviral Efficacy and Toxicity

a. Preparation of Human Foreskin Fibroblast Cells

Newborn human foreskins were obtained as soon as possible after circumcisions were performed and placed in minimal essential medium containing vancomycin, fungizone, penicillin, and gentamycin, at the usual concentrations, for four hours. The medium was then removed, the foreskin minced into small pieces and washed repeatedly until red cells were no longer present. The tissue was then trypsinized using trypsin at 0.25% with continuous stirring for 15 minutes at 37° C. in a CO₂ incubator. At the end of each 15 minute period the tissue was allowed to settle to the bottom of the flask. The supernatant containing cells was poured through sterile cheesecloth into a flask containing MEM and 10% fetal bovine serum. The flask containing the medium was kept on ice throughout the trypsinizing procedure. After each addition of cells, the cheesecloth was washed with a small amount of MEM containing serum. Fresh trypsin was added each time to the foreskin pieces and the procedure repeated until no more cells became available. The cell-containing medium was then centrifuged at 1000 RPM at 4° C. for ten minutes. The supernatant liquid was discarded and the cells resuspended in a small amount of MEM with 10% FBS. The cells were then placed in an appropriate number of 25 cm² tissue culture flasks. As cells became confluent and needed trypsinization, they were gradually expanded into larger flasks. The cells were kept on vancomycin and fungizone to passage four.

b. Cytopathic Effect Inhibition Assay HSV, HCMV, VZV

Low passage human foreskin fibroblast cells were seeded into 96-well tissue culture plates 24 h prior to use at a cell concentration of 2.5×10⁴ cells per mL in 0.1 mL of minimal essential medium (MEM) supplemented with 10% fetal bovine serum (FBS). The cells were then incubated for 24 h at 37° C. in a CO₂ incubator. After incubation, the medium was removed and 100 μl of MEM containing 2% FBS was added to all but the first row. In the first row, 125 μL of experimental drug was added in triplicate wells. Medium alone was added to both cell and virus control wells. The drug in the first row of wells was then diluted serially 1:5 throughout the remaining wells by transferring 25 mL using the Cetus Liquid Handling Machine. After dilution of drug, 100 μL of the appropriate virus concentration was added to each well, excluding cell control wells, which received 100 μL of MEM. For HSV-1 and HSV-2 assays, the virus concentration utilized was 1000 PFU's per well. For CMV and VZV assays, the virus concentration added was 2500 PFU per well. The plates were then incubated at 37° C. in a CO₂ incubator for three days for HSV-1 and HSV-2, 10 days for VZV, or 14 days for CMV. After the incubation period, media was aspirated and the cells stained with a 0.1% crystal violet solution for 30 minutes. The stain was then removed and the plates rinsed using tap water until all excess stain was removed. The plates were allowed to dry for 24 h and then read on a Skatron Plate Reader at 620 nm.

c. Plaque Reduction Assay for HSV-1 and HSV-2 Using Semi-Solid Overlay

Two days prior to use, HFF cells are plated into six-well plates and incubated at 37° C. with 5% CO₂ and 90% humidity. On the date of assay, the drug is made up at twice the desired concentration in 2×MEM and then serially diluted 1:5 in 2×MEM using six concentrations of drug. The initial starting concentration is usually 200 μg/mL down to 0.06 μg/mL. The virus to be used is diluted in MEM containing 10% FBS to a desired concentration which will give 20-30 plaques per well. The media is then aspirated from the wells and 0.2 mL of virus is added to each well in duplicate with 0.2 mL of media being added to drug toxicity wells. The plates are then incubated for one hour with shaking every fifteen minutes. After the incubation period, an equal amount of 1% agarose was added to an equal volume of each drug dilution. This will give final drug concentrations beginning with 100 μg/mL and ending with 0.03 μg/mL and a final agarose overlay concentration of 0.5%. The drug agarose mixture is applied to each well in 2 mL volume and the plates then incubated for three days, after which the cells were stained with a 1.5% solution of neutral red. At the end of 4-6 hr incubation period, the stain is aspirated, and plaques counted using a stereomicroscope at 10× magnification.

EC₅₀ (50% effective concentration) is the concentration required to inhibit viral cytopathogenicity by 50%.

IC₅₀ (50% inhibitory concentration) is the concentration required to inhibit cell proliferation by 50%.

Selective Index (S.I.) IC₅₀//EC₅₀

d. VZV Plaque Reduction Assay—Semi-Solid overlay.

The procedure is essentially the same as for the HSV plaque assay described above with two exceptions:

1. After addition of the drug, the plates are incubated for ten days.

2. On days three and six, an additional 1 mL overlay with equal amounts of 2×MEM and 1% agarose are added.

e. CMV Plaque Assay—Semi-Solid Overlay

The procedure again is nearly the same as for HSV with a few minor changes. The agarose used for both the initial overlay and the two subsequent overlays is 0.8% rather than 1%. The assay is incubated for 14 days with the additional 1 mL overlays being applied on days four and eight.

f. Plaque Reduction Assays Using Liquid Medium Overlay

The procedure for the liquid overlay plaque assay is similar to that using the agarose overlay. The procedure for adding the virus is the same as for the regular plaque assay. The drugs are made up in a concentration to be used in MEM with 2% FBS. The drugs are not made up at 2×concentration as in the previous assays but are made up at the desired concentration. For HSV-1 and HSV-2 assays, an antibody preparation obtained from Baxter Health Care Corporation is diluted 1:500 and added to the media that the drug is diluted in. For CMV and VZV, no antibody in the overlay is utilized. For the CMV assay, additional medium without new drug is added on day five and allowed to incubate for a total of 10 days. For VZV, additional medium is added on day five and incubated for a total of 10 days. At the end of the incubation period for all of the assays, 2 mL of 1:10 dilution of stock neutral is added to each well incubated for six hours. The liquid is then aspirated off and plaques enumerated using a stereomicroscope.

g. Screening and Confirmation Assays for EBV

1. Virus

There are two prototypes of infectious EBV. One is exemplified by the virus derived from supernatant fluids of the P3HR-1cell line. This cell line produces nontransforming virus that causes the production of early antigen (EA) after primary infection or superinfection of B cell lines. The other prototype is exemplified by the B-95-8 virus. This virus immortalized cord blood lymphocytes and induced tumors in marmosets. It does not, however, induce an abortive productive infection even in cell lines harboring EBV genome copies. The virus used in our assays is P3HR-1.

2. Cell Lines

Ramos is an exceptional B cell line derived from Burkitt's lymphoma tumor but containing no detectable EBV genome copies and is EBNA negative. Ramos/AW was obtained by in vitro infection of Ramos with the P3HR-1 virus and contains one resident EBV genome copy/cell. Raji is a Burkitt's lymphoma cell line containing 60 EBV genomes/cell, and will be the primary cell used for screening antiviral activity against EBV EA expression. Daudi is a low level producer that contains 152 EBV genome copies/cell. It spontaneously expresses EBV EA in 0.25%-0.5% of the cells. It will be used in follow-up studies to confirm activity. These cell lines respond to superinfection by EBV by expressing EA(D), EA(R), and VCA. All cell lines are maintained in RPMI-1640 medium supplemented by 10% FCS, L-glutamine and 100 μg/mL gentamicin. The cultures are fed twice weekly and the cell concentration adjusted to 3×10⁵/mL. The cells are kept at 37° C. in a humidified atmosphere with 5% CO₂.

3. Immunofluorescence Assays with Monoclonal Antibodies

Cells are infected with the P3HR-1 strain of EBV and the drugs to be tested are added after adsorption (45 minutes at 37° C.) and washing of the cell cultures. The cultures are incubated for two days in complete medium to allow viral gene expression. Following the 48 hr incubation period, the number of cells of each sample are counted and smears made. Monoclonal antibodies to the different EA components and VCA are then added to the cells incubated and washed. This is followed by a fluorescein conjugated rabbit anti-mouse Ig antibody; and the number of fluorescence positive cells in the smears are counted. The total number of cells in the cultures positive for EA or VCA are then calculated and compared.

h. Cell Proliferation Assay—Toxicity

Twenty-four hours prior to assay, HFF cells are seeded in 6-well plates at a concentration of 2.5×10⁴ cells per well in MEM containing 10% FBS. On the day of the assay, drugs are diluted serially in MEM containing 10% FBS at increments of 1:5 covering a range from 100 μg/mL to 0.03 μg/mL. For drugs that have to be solubilized in DMSO, control wells receive MEM containing 10% DMSO. The media from the wells is then aspirated and 2 mL of each drug concentration is then added to each well. The cells are then incubated in a CO₂ incubator at 37° C. for 72 h. At the end of this time, the media-drug solution is removed and the cells washed. One mL of 0.25% trypsin is added to each well and incubated until the cells start to come off of the plate. The cell media mixture is then pipetted up and down vigorously to break up the cell suspension, and 0.2 mL of the mixture is added to 9.8 mL of Isoton III and counted using a Coulter Counter. Each sample is counted three times with three replicate wells per sample.

i. MTT Assay for Cell Cytotoxicity

Twenty-four hours prior to assay, HFF cells are plated into 96-well plates at a concentration of 2.5×10⁴ cells per well. After 24 h, the media is aspirated and 125 mL of drug is added to the first row of wells and then diluted serially 1:5 using the automated Cetus Liquid Handling System in a manner similar to that used in the CPE assay. The plates are then incubated in a CO₂ incubator at 37° C. for seven days. At this time, each well receives 50 mL of 1 μg/mL solution of MTT in Dulbecco's Phosphate Buffered Saline. The plates are then incubated for an additional four hours. At this time, the media is removed and replaced with 100 μL of 0.04N hydrochloric acid in isopropanol. After shaking briefly, the plates are then read on a plate reader at 550 nm.

j. Neutral Red Uptake Assay—Toxicity

The procedure for plating cells and adding drug is the same as for the MTT Assay. After drug addition, the plates are incubated for seven days in a CO₂ incubator at 37° C. At this time the media/drug is aspirated and 200 μL/well of 0.01% neutral red in DPBS is added. This is incubated in the CO₂ incubator for one hour. The dye is aspirated and the cells are washed using a Nunc Plate Washer. After removing the DPBS wash, 200 μg/well of 50% EtOH/1% glacial acetic acid (in H₂O) is added. The plates are rotated for 15 minutes and the optical densities are read at 550 nm on a plate reader.

II. Assay Methods Of HBV & Influenza Virus: Analysis Of Potential Antiviral Agents Against HBV Replication In Cultures Of 2.2.15 Cells

A. Antiviral Assays

The protocol for assaying anti-HBV compounds in cultures of 2.2.15 cells can be briefly summarized as follows (Korba and Milman, 1991, Antiviral Res. 217:217). Chronically HBV-producing human liver cells (Acs, et al., 1987, PNAS 84:4641) are seeded into 24-well tissue culture plates and grown to confluence. Test compounds are then added daily for a continuous 9 day period. Culture medium (changed daily during the treatment period) is collected and stored for analysis of extracellular (virion) HBV DNA after 0, 3, 6, and 9 days of treatment. Treated cells are lysed 24 hours following day 9 of treatment for the analysis of intracellular HBV genomic forms. HBV DVA is then analyzed in a quantitative and qualitative manner for overall levels of HBV DNA (both extracellular and intracellular DNA) and the relative rate of HBV replication (intracellular DNA).

B. Toxicity Assays

The protocol for determining toxicity of compounds in cultures of 2.2.15 cells can be briefly summarized as follows. Cells of 2.2.15 were grown to confluence in 96-well flat-bottomed tissue culture plates and treated with compounds (in 0.2 mL culture medium/well) as described above. Four concentrations of each compound were assayed, each in triplicate cultures, in 3- to 10-fold steps. Untreated control cultures were maintained on each 96-well plate. On each 96-well plate, wells containing no cells were used to correct for light scattering. Toxicity was determined by the inhibition of the uptake of neutral red dye, determined by absorbance at 510 nm relative to untreated cells (Finter et al., 1969, J. Med. Chem 5:419), 24 hours following day 9 of treatment.

C. Assay Parameters

Both intracellular and extracellular HBV DNA are analyzed in order to (i) allow for verification of compound efficacy and (ii) provide possible data on the target site in the HBV replication pathway for the compound from examination of the pattern of viral replicative forms. The culture medium is changed daily during the treatment period to (i) prevent the buildup of potentially toxic metabolites derived from test compounds and (ii) provide an analysis of HBV virion production during discrete 24-hour intervals which enables a quantitative comparison of any effect on virion production.

The analysis of HBV DNA is performed using blot hybridization techniques (Southern and slot blot) and [³²P]-labeled HBV-specific probes. HBV DNA levels are measured by comparison to known amounts of HBV DNA standards applied to every nitrocellulose membrane (gel or slot blot). An AMBIS beta scanner, which measures the radioactive decay of the hybridized probes directly from the nitrocellulose membranes, is used for the quantitative analysis. Standard curves, generated by multiple analyses, are used to correlate CPM measurements made by the beta scanner with relative levels of target HBV DNA. The levels of HBV virion DNA released into the culture medium are analyzed by a slot blot hybridization procedure. HBV DNA levels are then compared to those at Day 0 to determine the effect of drug treatment.

A typical pattern of intracellular HBV DNA is displayed in the figure below (panel A, lanes 1 and 2). The levels of HBV DNA in each of three classes of viral genomic forms are individually quantitated in order to evaluate the replication status of the virus: episomal monomers, DNA replication intermediates [RI], and integrated HBV DNA.

The levels of RI and episomal monomers are used as an indicator of the relative level of HBV replication. Integrated HBV DNA is used to normalize the relative amounts of DNA in each lane because the levels of this class of HBV DNA would be expected to remain constant on a per cell basis. The type of changes in the intracellular HBV DNA patterns which are indicative of a decline in HBV replication are shown in lanes 3 and 4 of the figure. Inhibition of HBV DNA replication is indicated by the loss of RI without changes in the level of integrated HBV DNA.

III. Assays For Antiviral Activity Against Respiratory Viruses

A. Viruses Used in Primary Screen

a. Influenza A and B

Virus strains: A/Texas/36/91 (H1N1) (Source: Center for Disease Control and Prevention [CDC]), A/Beijing/2/92 (H₃N₂) (Source: CDC), B/Panama/45/90 (Source: CDC), A/NWS/33 (H1N1) (Source: American Type Culture Collection [ATCC]). (All but A/NWS/33 are tested in the presence of trypsin).

Cell lines: Madin Darby canine kidney (MDCK) cells.

b. Respiratory Syncytial Virus

Virus strain: Utah 89 (source: Utah State Diagnostic Laboratory)

Cell line: African green monkey kidney (MA-104) cells.

c. Parainfluenza Type 3 Virus

Virus strain: C243 (Source ATCC)

Cell line: African green monkey kidney (MA-104) cells.

d. Measles Virus

Virus strain: CC (Source: Pennsylvania State University)

Cell line: African green monkey kidney (BSC-1) cells.

e. Adenovirus Type 5

Virus strain: Adenoid 75 (Source ATCC)

Cell line: Human lung carcinoma (A549) cells.

B. Methods for Assay of Antiviral Activity

a. Inhibition of Viral Cytopathic Effect (CPE)

This test, run in 96-well flat-bottomed microplates, is used for the initial antiviral evaluation of all new test compounds. In this CPE inhibition test, seven one-half log₁₀ dilutions of each test compound will be added to 4 cups containing the cell monolayer; within 5 minutes, the virus is then added and the plate sealed, incubated at 37° C. and CPE read microscopically when untreated infected controls develop a 3 to 4+CPE (approximately 72 hr). A known positive control drug is evaluated in parallel with test drugs in each test. This drug is ribavirin for influenza, measles, respiratory syncytial and parainfluenza viruses, and HPMPA for adenovirus. The data are expressed as 50% effective (virus-inhibitory) concentrations (EC₅₀).

b. Increase in Neutral Red (NR) Dye Uptake

This test is run to validate the CPE inhibition seen in the initial test, and utilizes the same 96-well microplates after the CPE has been read. Neutral red is added to the medium; cells not damaged by virus take up a greater amount of dye, which is read on a computerized microplate autoreader. An EC₅₀ is determined from this dye uptake.

c. Decrease in Virus Yield

Compounds considered active by CPE inhibition and by NR dye uptake will be retested using both CPE inhibition, and, using the same plate, effect on reduction of virus yield by assaying frozen and thawed eluates from each cup for virus titer by serial dilution onto monolayers of susceptible cells. Development of CPE in these cells is the indication of presence of infectious virus. As in the initial tests, a known active drug is run in parallel as a positive control. The 90% effective concentration (EC₉₀), which is that test drug concentration that inhibits virus yield by 1 log₁₀, is determined from these data.

C. Methods For Assay Of Cytotoxicity

a. Visual Observation

In the CPE inhibition tests, two wells of uninfected cells treated with each concentration of test compound are run in parallel with the infected, treated wells. At the time CPE is determined microscopically, the toxicity control cells are also examined microscopically for any changes in cell appearance compared to normal control cells run in the same plate. These changes may be enlargement, granularity, cells with ragged edges, a filmy appearance, rounding, detachment from the surface of the well, or other changes. These changes are given a designation of T (100% toxic), P_{VH} (partially toxic-very heavy-80%), P_{H} (partially toxic-heavy-60%), P (partially toxic 40%), P_{S1} (partially toxic-slight-20%), or 0 (no toxicity-0%), conforming to the degree of cytotoxicity seen. A 50% cell inhibitory (cytotoxic) concentration (IC₅₀) is determined by regression analysis of these data.

b. Neutral Red Uptake

In the neutral red dye uptake phase of the antiviral test described above, the two toxicity control wells also receive neutral red and the degree of color intensity is determined spectrophotometrically. A neutral red IC50 (NR IC₅₀) is subsequently determined.

c. Viable Cell Count

Compounds considered to have significant antiviral activity in the initial CPE and NR tests are retested for their effects on cell growth. In this test, 12-well tissue culture plates are seeded with cells (sufficient to be approximately 20% confluent in the well) and exposed to varying concentrations of the test drug while the cells are dividing rapidly. The plates are then incubated in a CO₂ incubator at 37° C. for 72 hr, at which time the media-drug solution is removed and the cells washed. Trypsin is added to remove the cells, which are then counted using a Coulter cell counter. An IC₅₀ is then determined using the average of three separate counts at each drug dilution.

D. Data Analysis

Each test compound's antiviral activity is expressed as a selectivity index (SI), which is the IC₅₀ or IC₉₀ divided by the EC₅₀. Generally, an SI of 10 or greater is indicative of positive antiviral activity, although other factors, such as a low SI for the positive control, are also taken into consideration.

TABLE II
Antiviral Activities (EC₅₀, μg/mL) of Coumarin and Chromene Compounds

| | | | | | Respiratory | | |
|---|---|---|---|---|---|---|---|
| Compound | HIV | EBV | HBV | Measles | Syncitial | Rhinovirus | VZV |
| 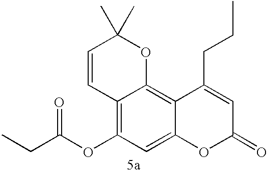 | >2 | ND | >10 | 0.4 (CPE) 0.3 (NR) 0.32 (virus yield) | 1 | ND | >4 |
| 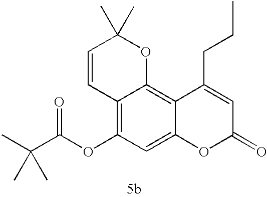 | >7 | 0.39 | >10 | 0.2 (CPE) 0.6 (NR) | ND | ND | >100 |
| 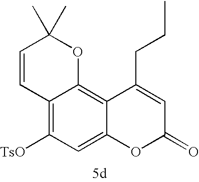 | 1.6 | 0.2 | >10 | >100 | >100 | >100 | >100 |
| 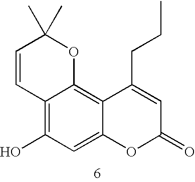 | ND | >50 | >10 | 0.5 (CPE) 0.6 (NR) | 1 | ND | 89 |
| 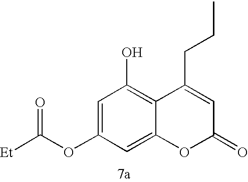 | >5 | >50 | >3 | 2 (CPE) 1 (NR) | 3 | >100 | >20 |
| 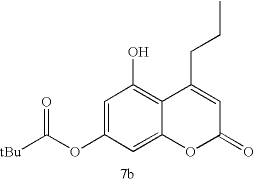 | ND | 1.1 | >3 | ND | ND | ND | 83 |
| 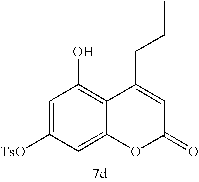 | >2 | 2.1 | 2.1 | 1 (CPE) 1 (NR) | 3 (CPE) 2 (NR) | 1 (CPE) 2 (NR) | >100 |
| 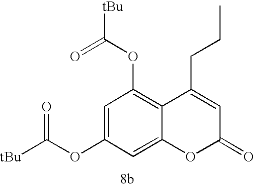 | 2.2 | 3.7 | >4 | 2 (CPE) 3 (NR) | >100 | 20 | 5.6 |
| 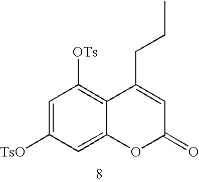 | >3 | >50 | >5 | 3 (CPE) 1 (NR) 3 (virus yield) | 2 (CPD) 1 (NR) | 2 (CPE) 1 (NR) | >20 |
| 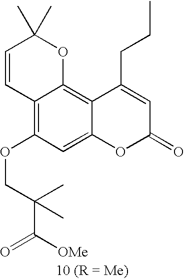 | 2 | 0.08 | >4 | 40 (CPE) 30 (NR) | >100 | 25 | >20 |
| 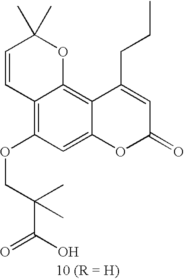 | >8 | ND | 2.6 | >100 (CPE) >100 (NR) | >100 | 10 (CPE) 1 (NR) | ND |
| 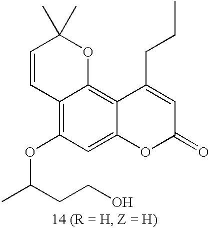 | 1.5 | ND | ND | ND | ND | ND | ND |
| 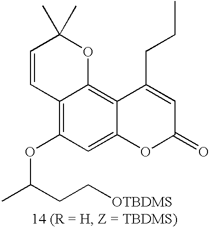 | >18 | ND | ND | ND | ND | ND | ND |
| 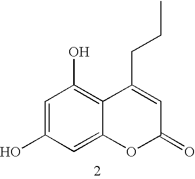 | ND | >50 | >2.2 | >100 (CPE) >100 (NR) | 6 | 27 | 5.9 |
| 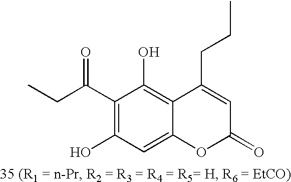 | >5 | 8.5 | 1 | 3 (CPE) 0.4 (NR) | | 2 (CPE) 5 (NR) | >4 |
| 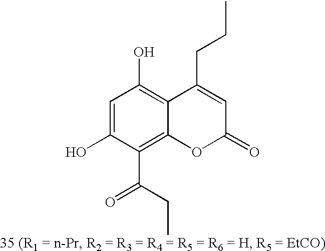 | ND | 0.33 | >3 | 10 (CPE) >100 (NR) | 2 (CPE) 4 (NR) | 30 (CPE) 28 (NR) | >0.8 |
| 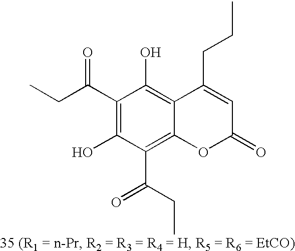 | >2 | 1.2 | >3 | >100 (CPE) >100 (NR) | 20 (CPE) 10 (NR) | 0.39 | >20 |
| 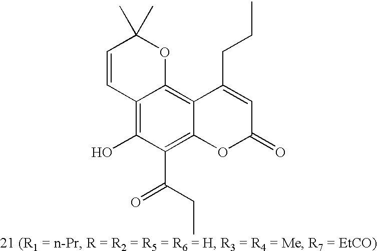 | >68 | 27 | >3 | 20 (CPE) >100 (NR) | 20 (CPE) 40 (NR) | ND | 16.1 |
| 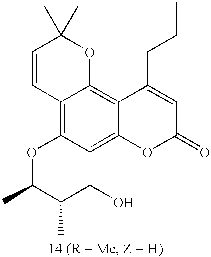 | >2 | ND | ND | ND | ND | ND | ND |
| 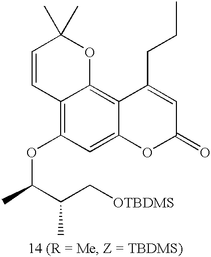 | 7.2 | ND | ND | ND | ND | ND | ND |
| 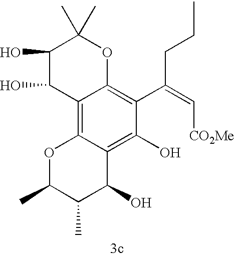 | >2 | >50 | >4 | 10 (CPE) 20 (NR) | 100 (CPE) >100 (NR) | 20 (CPE) 5 (NR) | >20 |
| 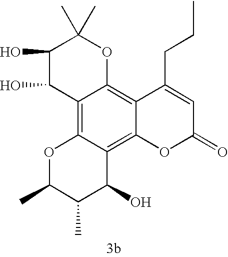 | >2.5 | >50 | >4 | 10 (CPE) 100 (NR) | >100 (CPE) 100 (NR) | 20 (CPE) 50 (NR) | >100 |
| 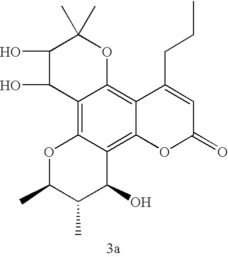 | >8 | 9.5 | >4 | 10 (CPE) 10 (NR) | >100 (CPE) >100 (NR) | 10 (CPE) 10 (NR) | >20 |
| 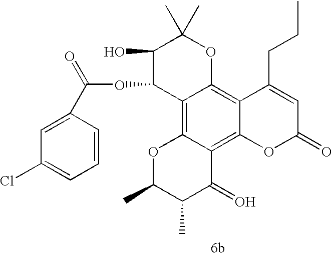 | >3 | 29.2 | 2.6 | 3 (CPE) 0.4 (NR) | 3 (CPE) 4 (NR) | 0.1 (CPE) 0.1 (NR) 4 (virus yield) | 3.5 |
| 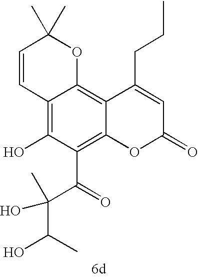 | >24 | ND | >4 | 20 (CPE) 40 (NR) | 50 (CPE) 40 (NR) | 0.5 (CPE) 1 (NR) | 19.9 |
| 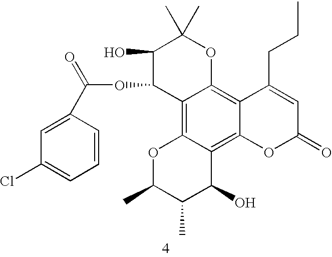 | 8.4 | ND | ND | ND | ND | ND | ND |

### REFERENCES

1a. Brookmeyer, R., Reconstruction and Future Trends of the AIDS Epidemic in the United States, Science, 1991, 253, 37-42.

b. Brain, M. M.; Heyward, W. L.; Curran, J. W., The Global Epidemiology of HIV Infection and AIDS, Annu. Rev. Microbiol., 1990, 44, 555-577.

2a. Weislow, O. S.; Kiser, R.; Fine, D. L.: Bader, J. Shoemaker, R. H.; Boyd, M. R., New Soluble-formazan Assay for HIV-1 Cytopathic Effects: Application to High-Flux Screening of Synthetic and Natural Products of AIDS-Antiviral Activity. J. Natl. Cancer Inst., 1989, 81, 577-586.

b. Mitsuya, H.; Yarchoan, R.; Broder, S., Molecular Targets for AIDS Therapy. Science, 1990, 249, 1533-1544.

c. Petteway, S. R., Jr.; Lambert, D. M.; Metcalf, B. W., The Chronically Infected Cells: A Target for the Treatment of HIV Infection and AIDS. Trends Pharmacol. Sci., 1991, 12, 28-34.

d. Richman, D. D., Antiviral Therapy of HIV Infection, Annu. Rev. Med., 1991, 42, 69-90.

e. Haden, J. W., Immunotherapy of Human Immunodeficiency Virus Infection. Trends Pharmacol Sci., 1991, 12, 107-111.

f. Huff, J. R., HIV Protease: A Novel Chemotherapeutic Target for AIDS. J. Med. Chem., 1991, 34, 2305-2314.

g. De Clercq, E., HIV Inhibitors Targeted at the Reverse Transcriptase. AIDS Research and Human Retroviruses, 1992, 8, 119-134.

3. Kashman, Y.; Gustafson, K. R.; Fuller, R. W.; Cardellina, J. H., II; McMahon; J. B.; Currens, M. J.; Buckheit, R. W., Jr.; Hughes, S. H.; Cragg, G. M.; Boyd, M. R., The Calanolides, a Novel HIV-Inhibitory Class of Coumarin Derivatives from the Tropical Rainforest Tree, Calophyllum lanigerum. J. Med. Chem. 1992, 35, 2735-2743.

4. Hoofnagle, J. H. Chronic hepatitis B, N. Engl. J. Med. 1990, 323, 337-339.

5. Martin, P. and Friedman, L. S. In Innovations in Antiviral Development and the Detection of Virus Infections; T. M. Block; D. Junkind; R. L. Crowell; M. Dension; L. R. Walsh, Ed.; Plenum Press: New York, 1992, 111-120.

6. Aach, R. D. The treatment of chronic type B viral hepatitis. Ann. Intern. Med. 1988, 109, 88-91.

7. Alexander, G. J.; Brahm, J.; Fagan, E. A.; Smith, H. M.; Daniels, H. M.; Eddleston, A. L.; Williams, R., Loss of HBSAg with interferon therapy in chronic hepatitis B virus infection. Lancet 1987, ii, 66-69.

8. Hoofnagle, J. H.; Di Bisceglie, A. M. Antiviral therapy of viral hepatitis. In Antiviral Agents and Viral Diseases of Man; G. J. Galasso; R. J. Whiteley; T. C. Merigan, Ed; Raven Press: New York, 1972, 415-457.

9. Yokosuka, O.; Omata, O. M.; Imazeki, F.; Okauda, K.; Summers, J. Changes of hepatitis B virus DNA in liver and serum caused by recombinant leukocyte interferon treatment: analysis of intrahepatic replicative hepatitis B virus DNA. Hepatology 1985, 5, 728-734.

10. Doong, S. L.; Tsai, C. H.; Schinazi, R. F.; Liota, D. C.; Cheng, Y. C. Inhibition of the replication of hepatitis B virus in vitro by 2′,3′-dideoxy-3′-thiacytidine and related analogues. Pro. Natl. Acad. Sci. USA 1991, 88, 8495-99.

11. Schlam, S. W.; de Man, R. A.; Heijtink, R. A.; Niesters, G. M. New nucleoside analogues for chronic hepatitis B. J. Hepatalogy 1995, 22, 52-56.

12. van Leeuwen R.; Katlama, C.; Kitchen, V.; Boucher, C. A. B.; Tubiana, R.; McBride, M.; Ingrand, D.; Weber, J.; Hill, A.; McDade, H.; Danner, S. A. Evaluation of safety and efficacy of 3TC (Lamivudine) in patients with asymptomatic or mildly symptomatic human immunodeficiency virus infection: A phase I/II study. J. Inf. Dis. 1995,171, 1166-71.

13. Kaplan, M. M.; Webster, R. G. The epidemiology of influenza, Sci. Am., 1977, 236 (6), 88-105.

14. Hoffman, C. E. Amantadine HCl and related compounds. In Selective Inhibitors of Viral Functions; Carter, W. A., Ed.; CRC Press: Cleveland, 1973, 199.

15. Dolin, R.; Reichman, R. C.; Madore, H. P.; Maynard, R.; Lindon, P. M.; Webber-Jones, J. A controlled trial of amantadine and rimandatine in the prophylaxis of influenza A infections. N. Engl. J. Med. 1982, 307, 580-584.

16. Oxford, J. S.; Galbraith, A. Anti-influenza virus activity of amantadine: A selective review of laboratory and clinical data: In Viral Chemotherapy; Shugar D. Ed.; Pergamon Press, 1985, 169-254.

17. Couch, R. B.; Jackson, G. G. Antiviral agents in influenza-Summary of influenza workshop VIII. J. Infect. Dis. 1976, 134, 516-527.

18. Bryson, Y. J.; Monahan, C.; Pollack, M.; Shields, W. D. A prospective double-blind study of side effects associated with the administration of amantadine for influenza A virus prophylaxis. J. Infect. Dis. 1980, 141, 543-547.

19. Tsunoda, A.; Maasab, H. H.; Cochran, K. W.; Eveland, W. C. Antiviral activity of α-methyl-1-adamantane methylamine hydrochloride. Antimicrob. Agents Chemother. 1966, 553.

20. Tisdale, M.; Bauer, D. J. The relative potencies of anti-influenza compounds. Ann. N.Y. Acad. Sci. 1977, 284, 254-263.

21. Degelau, J; Somani, S. K.; Cooper, S. L.; Guay, D. R. P.; Crossley, K. B. Amantadine-resistant influenza A in a nursing facility. Arch. Intern. Med. 1992, 152, 390-392.

22. Hayden, F. G.; Belshe, R. B.; Clover, R. D.; Hay, A. J.; Oakers, M. G.; Soo, W. Emergence and apparent transmission of rimantadine-resistant influenza virus in families. N. Engl. J. Med. 1989, 321, 1696-1702.

23. Mast, E. E.; Harmon, M. W.; Gravenstein, S.; Wu, S. P.; Arden, H. H.; Circo, R.; Tyszka, G.; Kendal, A. P.; Davis, J. P. Emergence and possible transmission of amantadine-resistant viruses during nursing. home outbreaks of influenza A (H₃N₂). Am J. Epidemiol. 1992, 134, 988-997.

24. Hayden, F. G.; Couch, R. B. Clinical and epidemiological importance of influenza A viruses resistant to amantadine and rimantadine. Rev. Med. Virol., 1992, 2, 89-96.

25. Kimberlin, D. W.; Crampacker, C. S.; Straus, S. E.; Biron, K. K.; Drew, W. L.; Hayden, F. G.; McKinlay, M.; Richman, D. D.; Whitley, R. J. Antiviral resistance in clinical practice. Antiviral Res., 1995, 26, 423-438.

26. Knight, V.; Gilbert, B. E. Ribavirin aerosol treatment of influenza. In Infectious Disease Clinics of North America, Vol 1.; Moellering, Jr. Ed.; 1987, 441-57.

27. Ray, C. G.; Icenogle, T. B.; Minnich, L. L; Copeland, J. G.; Grogan, T. M. The use of intravenous ribavirin to treat influenza virus-associated acute myocarditis. J. Infect Dis., 1989, 159, 829-836.

28. Hoover, D. R.; Saah, A. J.; Bacellar, H.; Phair, J.; Detels, R.; Anderson, R.; Kaslow, R. A. Clinical manifestations of AIDS in the Era of Pneumocysis Prophylaxis. N. Eng. J. Med. 1993, 329, 1922-1926.

29. Studies of the Ocular Complications of AIDS Research Group, AIDS clinical Trials Group. Mortality in Patients with the Acquired Immunodeficiency Syndrome Treated with Either Foscarnet or Ganciclovir for Cytomegaloviruse Retinitis. N. Eng. J. Med. 1992, 326, 213-220.

30. Gelb, C. D. In The Human Herpesviruses, New York, 1993, 288-300.

31. Dellamonica, P. et al. Clin. Pharmacol. 1991, 10, 301

32. Smith, J. Strategies against Herpes Simplex Virus. Intl. Antiviral News 1997, 5, 223-225.

33. Patil, A. D.; Freyer, A. J.; Eggleston, D. S.; Haltiwanger, R. C.; Bean, M. F.; Taylor, P. B.; Caranfa, M. J.; Breen, A. L.; Bartus, H. R.; Johnson, R. K.; Hertzberg, R. P.; Westley, J. W. The Inophyllums, Novel Inhibitors of HIV-1 Reverse Transcriptase Isolated from the Malaysian Tree, Calophyllum inophyllum Linn. J. Med. Chem. 1993, 36, 4131-4138.

34. Fuller, R. W.; Bokesch, H. R.; Gustafson, K. R.; McKee, T. C.; Cardellina, J. H, II; McMahon, J. B.; Cragg, G. M.; Soejarto, D. D.; Boyd, M. R. HIV-Inhibitory Coumarins from Latex of the Tropical Rainforest Tree Calophyllum teysmannii var. inophylloide. Bioorg. & Med. Chem. Lett. 1994, 4, 1961-1964.

35. Gustafson, K. R.; Bokesch, H. R.; Fuller, R. W.; Cardellina, J. H, II; Kadushin, M. R.; Soejarto, D. D.; Boyd, M. R. Calanone, a Novel Coumarin from Calophyllum teysmannii. Tetrahedron Lett. 1994, 35, 5821-5824.

36. Dharmaratne, H. R. W.; Wanigasekera, W. M. A. P.; Mata-Greenwood, E.; Pezzuto, J. M. Ihibition of Human Immunodeficiency Virus Type 1 Reverse Transcriptase Activity by Cordatolides Isolated from Calophyllum cordato-oblongum. Planta Med. 1998, 64, 460-461.

37. McKee, T. C.; Convington, C. D.; Fuller, R. W.; Bokesch, H. R.; Young, S.; Cardellina, J. H., II; Kadushin, M. R.; Soejarto, D. D.; Stevens, P. F.; Cragg, G. M.; Boyd, M. R. Pyranocoumarins from Tropical Species of the Genus Calophyllum: A Chemotaxonomic Study of Extracts in the National Cancer Institute Collection. J. Nat. Prod. 1998, 61, 1252-1256.

38. Spino, C.; Dodier, M.; Sotheeswaran, S. Anti-HIV Coumarins from Calophyllum Seed Oil. Bioorg. Med. Chem. Lett. 1998, 8, 3475-3478.

39. Galinis, D. L.; Fuller, R. W.; McKee, T. C.; Cardellina, J. H, II; Gulakowski, R. J.; McMahon, J. B.; Boyd, M. R. Structure-Activity Modifications of the HIV-1 Inhibitors (+)-Calanolide A and (−)-Calanolide B. J. Med. Chem. 1996, 39, 4507-4510.

40. Zhou, C. M.; Wang, L.; Zhao, Z. Z. Synthesis of 11-Demethyl and 6,6,11-Demehtyl Calanolide A. Chinese Chem. Lett. 1997, 8, 859-860.

41. Zembower, D. E.; Liao, S.; Flavin, M. T.; Xu, Z.-Q.; Stup, T. L.; Buckheit, R. W., Jr.; Khilevich, A.; Mar, A. A.; Sheinkman, A. K. Structural Analogues of the Calanolide Anti-HIV Agents. Modification of the trans-10,11-Dimethyldihydropyran-12-ol Ring (Ring C). J. Med. Chem. 1997, 40, 1005-1017.

42. Xu, Z.-Q.; Kern, E. R.; Westbrook, L.; Allen, L. B.; Buckheit, R. W., Jr.; Tseng, C. K.-H.; Jenta, J.; Flavin, M. T. Plant-Derived and Semisynthetic Calanolide Compounds with In Vitro Activity against Both Human Immunodeficiency Virus Type 1 and Human Cytomegalovirus. Antiviral Chem. Chemotherapy, 2000, 11, 23-29.

43. Frank, P.; Flavin, M. T.; Roca-Acin, J.; Xu, Z.-Q. Safety Assessment of (+)-Calanolide A, A Naturally Occurring Anti-HIV Agent. 4th Conference on Retroviruses ans Opportunistic Infections, Jan. 22-26, 1997, Washington, D. C., Abstract No. 225.

44. Jon Ruckle, Jeremey Giltner, Terri Creagh, Bipul Dutta, Dwain Tolbert and Ze-Qi Xu, Clinical Safety and Pharmacokinetics of (+)-Calanolide A, A Naturally Occuring NNRTI in Normal Healthy and HIV-Infected Volunteers. 5^{th} Conference on Retroviruses and Opportunistic Infections, January 31-Feb. 4, 1999, Chicago, Ill., Abstract No. 606.

45. Desai, R. D.; Parghi, J. V. Heterocyclic coumpounds. Part XXXI. The Synthesis of Partially Methylated Ethers of Polyhydroxy Coumarins. Indian Chem. Soc. 1956, 33, 661-664.

46. Fox, M. E.; Meek, G. A. Process for the Preparation of Calanolide Procedures. WO 0064902, 2000.

47. Kucherenko, A.; Flavin, M. T.; Boulanger, W. A.; Khilevich, A.; Shone, R. L.; Rizzo, J. D.; Sheinkman, A. K.; Xu, Z.-Q. Novel Approach for Synthesis of (±)-Calanolide A and Its Anti-HIV Activity. Tetrahedron Lett. 1995, 36, 5475-5478.

48. Flavin, M. T.; Rizzo, J. D.; Khilevich, A.; Kucherenko, A.; Sheinkman, A. K.; Vilaychack, V.; Lin, L.; Chen, W.; Greenwood, E. M.; Pengsuparp, T.; Pezzuto, J. M.; Hughes, S. H.; Flavin, T. M.; Cibulski, M.; Boulanger, W. A.; Shone, R. L.; Xu, Z.-Q. Synthesis, Chromatographic Resolution and Anti-HIV Activity of (±)-Calanolide A and Its Enantiomers. J. Med. Chem. 1996, 39, 1303-1313.

49. Chenera, B.; West, M. L.; Finkelstein, J. A.; Dreyer, G. B. Total Synthesis of (±)-Calanolide A, a Non-nucleoside Inhibitor of HIV-1 Reverse Transcriptase. i J. Org. Chem. 1993, 58, 5605-5606.

50. Bell, D.; Davies, M. R.; Geen, G. R.; Mann, I. S. Copper(I) Iodide: A Catalyst for the Improved Synthesis of Aryl Propargyl Ethers. Synthesis 1995, 707-712.

51. Games, D. E.; Haskins, N. J. Synthesis of Some Dimethylpyrano- and 3-Methylbut-2-enyl-4-phenyl- and 4-n-propyl-coumarins. J. Chem. Soc., Chem. Commun., 1971, 1005-1006.

52. Petit, Y.; Sanner, C.; Larcheveque, M. Synthesis 1998, 538.

53. Frater, G.; Muller, U.; Gunther, W. Tetrahedron 1984, 40, 1269.

54. Kikukawa, T.; Imaida, M.; Tai, A. Bull. Chem. Soc. Jpn. 1984, 57, 1954.

55. Nerz-Stormes, M.; Thornton, E. R. J. Org. Chem. 1991, 56, 2489.

We claim:

1. A method for treating or preventing viral infections comprising administering to a subject in need of anti-viral treatment or prevention an anti-viral effective amount of a compound having the formula I, or a pharmaceutically acceptable salt thereof: 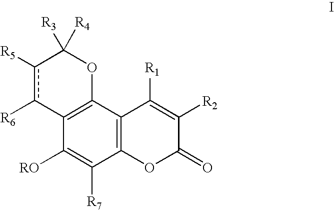
wherein R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen;
R₂ is H, halogen, hydroxyl, amino, thio, cyano, C₁₋₆ alkyl, aryl-C₁₋₆ alkyl, mono- or poly-fluorinated C₁₋₆ alkyl, aryl or heterocycle; or
R₁ and R₂ together can form a 5-7 membered saturated or unsaturated cyclic ring or heterocyclic ring;
R₃ and R₄ are independently selected from the group consisting of H, C₁₋₆ alkyl, aryl-C₁₋₆ alkyl, mono- or poly-fluorinated C₁₋₆ alkyl, aryl or heterocycle; or R₃ and R₄ together can form a 5-7 membered saturated or unsaturated cyclic ring or heterocyclic ring;
R₅ and R₆ are independently selected from the groups consisting of H, halogen, hydroxyl, amino, nitro, thio, cyano, azido, C₁₋₆ alkyl, aryl-C₁₋₆ alkyl, mono- or poly-fluorinated C₁₋₆ alkyl, aryl, or heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen;
R₇ is H, halogen, hydroxyl, amino, nitro, thio, cyano, azido, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, —C(O)R₈, —SO₂R₈, —P(O)(OR₈)₂, —P(O)(OR₈)(OR₉), —R₈C(O)R₉, —R₈SO₂R₉, or —R₈P(O)(OR₉)₂.
R is H, C₁₋₆ alkyl, aryl-C₁₋₆ alkyl, mono- or poly-fluorinated C₁₋₆ alkyl, aryl, heterocycle, —C(O)R₈, —SO₂R₈, —P(O)(OR₈)₂, —P(O)(OR₈)(OR₉), —R₈C(O)R₉ or —R₈SO₂R₉, —R₈P(O)(OR₉)₂, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; and
R₈ and R₉ are independently selected from the groups consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C¹⁻⁸ alkyl, nitro, thio, cyano, azido, and halogen.

2. The method of claim 1 wherein R₁ is propyl; R₂ and R₅ are H; R₃ and R₄ are methyl; and
R is

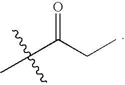

3. The method of claim 1 wherein R₁ is propyl; R₂ and R₅ are H; R₃ and R₄ are methyl; and
R is

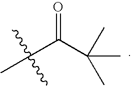

4. The method of claim 1 wherein R₁ is propyl; R₂ and R₅ are H; R₃ and R₄ are methyl; and R is p-toluenesulfonyl.

5. The method of claim 1 wherein R₁ is propyl; R₂ and R₅ are H; R₃ and R₄ are methyl; and R is H.

6. The method of claim 1 wherein R₁ is propyl; R₂ and R₅ are H; R₃ and R₄ are methyl; and
R is

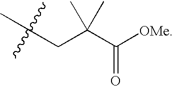

7. The method of claim 1 wherein R₁ is propyl; R₂ and R₅ are H; R₃ and R₄ are methyl; and
R is

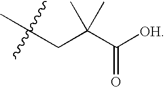

8. The method of claim 1 wherein R₁ is propyl; R₂ and R₅ are H; R₃ and R₄ are methyl; and
R is

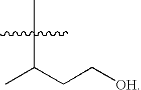

9. The method of claim 1 wherein R₁ is propyl; R₂ and R₅ are H; R₃ and R₄ are methyl; and
R is

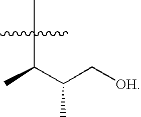

10. The method of claim 1 wherein R₁ is propyl; R and R₂ and R₅ are H; R₃ and R₄ are methyl; and
R₆ is

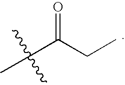

11. A method for treating or preventing viral infections comprising administering to a subject in need of anti-viral treatment or prevention an anti-viral effective amount of a compound having the formula II, or a pharmaceutically acceptable salt thereof: 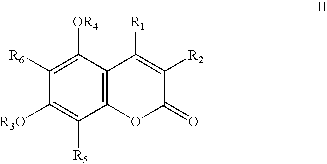
wherein R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen;
R₂ is H, halogen, hydroxyl, amino, thio, cyano, C₁₋₆ alkyl, aryl-C₁₋₆ alkyl, mono- or poly-fluorinated C₁₋₆ alkyl, aryl, or heterocycle; or
R₁ and R₂ together can form a 5-7 membered saturated or unsaturated cyclic ring or heterocyclic ring;
R₃ and R₄ are independently selected from the group consisting of H, C₁₋₆ alkyl, aryl-C₁₋₆ alkyl, mono- or poly-fluorinated C₁₋₆ alkyl, aryl, heterocycle, —C(O)R₇, —SO₂R₇, —P(O)(OR₇)₂, —P(O)(OR₇)(OR₈), —R₇C(O)R₈, —R₇SO₂R₈, or —R₇P(O)(O)₂, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C¹⁻⁸ alkyl, di(C₁₋₆ alkyl)-amino-C₁₋₈ alkyl, nitro, thio, cyano, azido, and halogen;
R₅ and R₆ are independently selected from the group consisting of H, halogen, hydroxyl, amino, nitro, thio, cyano, azido, C₁₋₆ alkyl, aryl-C₁₋₆ alkyl, mono- or poly-fluorinated C₁₋₆ alkyl, aryl, heterocycle, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, —C(O)R₇, —SO₂R₇, —P(O)(OR₇)₂, —P(O)(OR₇)(OR₈), —R₇C(O)R₈, —R₇SO₂R₈, and —R₇P(O)(OR₈)₂—;
R₇ and R₈ are independently selected from the group consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, and heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen.

12. The method of claim 11 wherein R₁ is propyl; R₂, R₃, R₄, and R₅ are H; and
R is

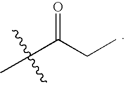

13. The method of claim 11 wherein R₁ is propyl; R₂, R₃, R₄, and R₅ are H; and
R is

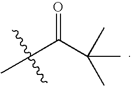

14. The method of claim 11 wherein R₁ is propyl; R₂, R₃, R₄, and R₅ are H; and R is p-toluenesulfonyl.

15. The method of claim 11 wherein R₁ is propyl; R₂, R₄, and R₅ are H; and
R₃ and R are

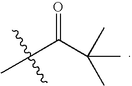

16. The method of claim 11 wherein R₁ is propyl; R₂, R₄, and R₅ are H; and R₃ and R are p-toluene-sulfonyl.

17. The method of claim 11 wherein R₁ is propyl; and R, R₂, R₃, R₄, and R₅ are H.

18. The method of claim 11 wherein R is propyl; R, R₂, R₃, and R₅ are H; and R₄ is

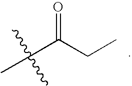

19. The method of claim 11 wherein R₁ is propyl; R, R₂, R₃, and R₄ are H; and R₅ is

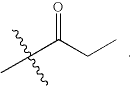

20. The method of claim 11 wherein R₁ is propyl; R, R₂, and R₃ are H; and R₄ and R₅ are

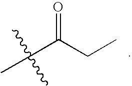

21. A method for treating or preventing viral infections comprising administering to a subject in need of anti-viral treatment or prevention an anti-viral effective amount of compound having the formula III, or a pharmaceutically acceptable salt thereof: 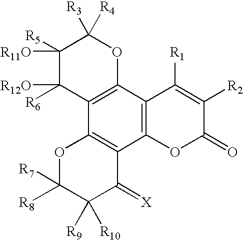
wherein R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen;
R₂ is H, halogen, hydroxyl, amino, thio, cyano, C₁₋₆ alkyl, aryl-C₁₋₆ alkyl, mono- or poly-fluorinated C₁₋₆ alkyl, aryl, or heterocycle; or
R₁ and R₂ together can form a 5-7 membered saturated or unsaturated cyclic ring or heterocyclic ring;
R₅ and R₆ are independently selected from the group consisting of H, C₁₋₆ alkyl, aryl-C₁₋₆ alkyl, mono- or poly-fluorinated C₁₋₆ alkyl, aryl, and heterocycle; or R₅ and R₆ together can form a 5-7 membered saturated or unsaturated cyclic ring or heterocyclic ring;
R₃, R₄, R₇, R₈, R₉, and R₁₀ are independently selected from the group consisting of H, halogen, hydroxyl, amino, nitro, thio, cyano, azido, C₁₋₆ alkyl, aryl-C₁₋₆ alkyl, mono- or poly-fluorinated C₁₋₆ alkyl, —C(O)R₁₃, —SO₂R₁₃, —R₁₃C(O)R₁₄, —R₁₃SO₂R₁₄, aryl, and heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; or
any of R₃ and R₄ together, R₇ and R₈ together, or R₉ and R₁₀ together, can form a 5-7 membered saturated or unsaturated cyclic ring or heterocyclic ring;
R₁₁ and R₁₂ are H, C₁₋₆ alkyl, aryl-C₁₋₆ alkyl, mono- or poly-fluorinated C₁₋₆ alkyl, —C(O)R₁₃, —SO₂R₁₃, —P(O)(OR₁₃)₂, —R₁₃C(O)R₁₄, —R₁₃SO₂R₁₄, —R₁₃P(O)(OR₁₄)₂, amino acid, aryl, or heterocycle; wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; and
R₁₃ and R₁₄ are independently selected from the group consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl) amino-C₁₋₈ alkyl, cyclohexyl, aryl, and heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; and
X is H, halogen, OH, O, SH, NH₂, NHOH, ═NOH, or NR₁₁R₁₂ wherein R₁₁ and R₁₂ are defined as above, or R₁₁ and R₁₂ together form a 5-7 membered saturated or unsaturated cyclic ring or heterocyclic ring.

22. A composition comprising an amount effective to inhibit viral infection of a compound of formula I, II, or III or a pharmaceutically acceptable salt thereof, in combination with a pharmaceutically acceptable carrier: 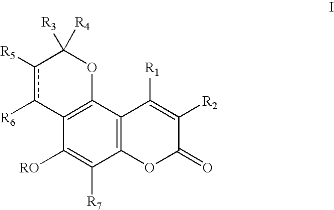
wherein R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen;
R₂ is H, halogen, hydroxyl, amino, thio, cyano, C₁₋₆ alkyl, aryl-C₁₋₆ alkyl, mono- or poly-fluorinated C₁₋₆ alkyl, aryl or heterocycle; or
R₁ and R₂ together can form a 5-7 membered saturated or unsaturated cyclic ring or heterocyclic ring;
R₃ and R₄ are independently selected from the group consisting of H, C₁₋₆ alkyl, aryl-C₁₋₆ alkyl, mono- or poly-fluorinated C₁₋₆ alkyl, aryl or heterocycle; or R₃ and R₄ together can form a 5-7 membered saturated or unsaturated cyclic ring or heterocyclic ring;
R₅ and R₆ are independently selected from the groups consisting of H, halogen, hydroxyl, amino, nitro, thio, cyano, azido, C₁₋₆ alkyl, aryl-C₁₋₆ alkyl, mono- or poly-fluorinated C₁₋₆ alkyl, aryl, or heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen;
R₇ is H, halogen, hydroxyl, amino, nitro, thio, cyano, azido, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, —C(O)R₈, —SO₂R₈, —P(O)(OR₈)₂, —P(O)(OR₈)(OR₉), —R₈C(O)R₉, —R₈SO₂R₉, or —R₈P(O)(OR₉)₂.
R is H, C₁₋₆ alkyl, aryl-C₁₋₆ alkyl, mono- or poly-fluorinated C₁₋₆ alkyl, aryl, heterocycle, —C(O)R₈, —SO₂R₈, —P(O)(OR₈)₂, —P(O)(OR₈)(OR₉), —R₈C(O)R₉ or —R₈SO₂R₉, —R₈P(O)(OR₉)₂, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; and
R₈ and R₉ are independently selected from the groups consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; 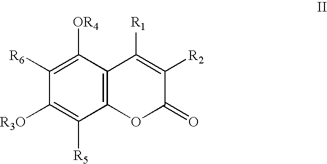
wherein R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen;
R₂ is H, halogen, hydroxyl, amino, thio, cyano, C₁₋₆ alkyl, aryl-C₁₋₆ alkyl, mono- or poly-fluorinated C₁₋₆ alkyl, aryl, or heterocycle; or
R₁ and R₂ together can form a 5-7 membered saturated or unsaturated cyclic ring or heterocyclic ring;
R₃ and R₄ are independently selected from the group consisting of H, C₁₋₆ alkyl, aryl-C₁₋₆ alkyl, mono- or poly-fluorinated C₁₋₆ alkyl, aryl, heterocycle, —C(O)R₇, —SO₂R₇, —P(O)(OR₇)₂, —P(O)(OR₇)(OR₈), —R₇C(O)R₈, —R₇SO₂R₈, or —R₇P(O)(OR₈)₂, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)-amino-C₁₋₈ alkyl, nitro, thio, cyano, azido, and halogen;
R₅ and R₆ are independently selected from the group consisting of H, halogen, hydroxyl, amino, nitro, thio, cyano, azido, C₁₋₆ alkyl, aryl-C₁₋₆ alkyl, mono- or poly-fluorinated C₁₋₆ alkyl, aryl, heterocycle, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, —C(O)R₇, —SO₂R₇, —P(O)(OR₇)₂, —P(O)(OR₇)(OR₈), —R₇C(O)R, —R₇SO₂R₈, and —R₇P(O)(OR₈)₂;
R₇ and R₈ are independently selected from the group consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₆ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, and heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; 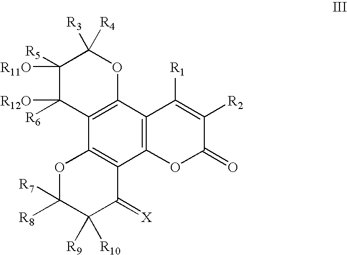
wherein R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen;
R₂ is H, halogen, hydroxyl, amino, thio, cyano, C₁₋₆ alkyl, aryl-C₁₋₆ alkyl, mono- or poly-fluorinated C₁₋₆ alkyl, aryl, or heterocycle; or
R₁ and R₂ together can form a 5-7 membered saturated or unsaturated cyclic ring or heterocyclic ring;
R₅ and R₆ are independently selected from the group consisting of H, C₁₋₆ alkyl, aryl-C₁₋₆ alkyl, mono- or poly-fluorinated C₁₋₆ alkyl, aryl, and heterocycle; or R₅ and R₆ together form a 5-7 membered saturated or unsaturated cyclic ring or heterocyclic ring;
R₃, R₄, R₇, R₈, R₉, and R₁₀ are independently selected from the group consisting of H, halogen, hydroxyl, amino, nitro, thio, cyano, azido, C₁₋₆ alkyl, aryl-C₁₋₆ alkyl, mono- or poly-fluorinated C₁₋₆ alkyl, —C(O)R₁₃, —SO₂R₁₃, —R₁₃C(O)R₁₄, —R₁₃SO₂R₁₄, aryl, and heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; or
any of R₃ and R₄ together, R₇ and R₈ together, or R₉ and R₁₀ together, can form a 5-7 membered saturated or unsaturated cyclic ring or heterocyclic ring;
R₁₁ and R₁₂ are H, C₁₋₆ alkyl, aryl-C₁₋₆ alkyl, mono- or poly-fluorinated C₁₋₆ alkyl, —C(O)R₁₃, —SO₂R₁₃, —P(O)(OR₁₃)₂, —R₁₃C(O)R₁₄, —R₁₃SO₂R₄, —R₁₃P(O)(OR₁₄)₂, amino acid, aryl, or heterocycle; wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; and
R₁₃ and R₁₄ are independently selected from the group consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, and heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; and
X is H, halogen, OH, O, SH, NH₂, NHOH, ═NOH, or NR₁₁R₁₂ wherein R₁₁, and R₁₂ are defined as above, or R₁₁ and R₁₂ together form a 5-7 membered saturated or unsaturated cyclic ring or heterocyclic ring.

23. The composition according to claim 22, further comprising an amount effective to inhibit viral infection with at least one other pharmaceutical agent.

24. A method for designing or selecting a molecule effective to inhibit a viral infection comprising:
a) contacting a virus or a viral enzyme with an amount of a compound of formula I, II, or III;
b) measuring the viability of the virus or activity of the viral enzyme, relative to a control virus or control viral enzyme that is not contacted with an amount of a compound of formula I, II, or III;
c) selecting the compounds of formula I, II, or III that demonstrated the greatest potency in inhibition of the virus replication or the its enzymatic activity; and
d) identifying one or more common structural elements in the compounds selected in (c), that differ from compounds not selected in (c);
wherein the designed or selected molecule effective to inhibit a viral infection comprises the one or more structural elements identified in (d).

25. The method of any of claims 1, 11, or 21, wherein the viral infection is related to infection by a virus selected from the group consisting of: human immunodeficiency virus, Epstein-Barr Virus, Hepatitis B Virus, measles, respiratory syncytial virus, rhinovirus, and varicella zoster virus.

26. A compound having the formula I: 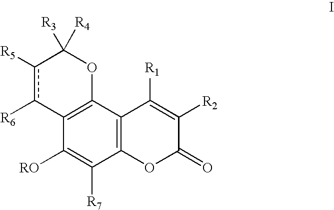
wherein R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen;
R₂ is H, halogen, hydroxyl, amino, thio, cyano, C₁₋₆ alkyl, aryl-C₁₋₆ alkyl, mono- or poly-fluorinated C₁₋₆ alkyl, aryl, or heterocycle; or
R₁ and R₂ together can form a 5-7 membered saturated or unsaturated cyclic ring or heterocyclic ring;
R₃ and R₄ are independently selected from the group consisting of H, C₁₋₆ alkyl, aryl-C₁₋₆ alkyl, mono- or poly-fluorinated C₁₋₆ alkyl, aryl or heterocycle; or R₃ and R₄ together form a 5-7 membered saturated or unsaturated cyclic ring or heterocyclic ring;
R₅ and R₆ are independently selected from the groups consisting of H, halogen, hydroxyl, amino, nitro, thio, cyano, azido, C₁₋₆ alkyl, aryl-C₁₋₆ alkyl, mono- or poly-fluorinated C₁₋₆ alkyl, aryl, and heterocycle, wherein aryl or heterocycle may each independently be unsubstituted or substituted with one or more from the group consisting of: C₁₋₆ alkyl, C-6 alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen;
R₇ is H, halogen, hydroxyl, amino, nitro, thio, cyano, azido, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, —C(O)R₈, —SO₂R₈, —P(O)(OR₈)₂, —P(O)(OR₈)(OR₉), —R₈C(O)R₉, —R₈SO₂R₉, or —R₈P(O)(OR₉)₂.
R is H, C₁₋₆ alkyl, aryl-C₁₋₆ alkyl, mono- or poly-fluorinated C₁₋₆ alkyl, aryl, heterocycle, —C(O)R₈, —SO₂R₉, —P(O)(OR₈)₂, —P(O)(OR₈)(OR₉), —R₈C(O)R₉ or —R₈SO₂R₉, —R₈P(O)(OR₉)₂, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; and
R₈ and R₉ are independently selected from the groups consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, and heterocycle, wherein aryl or heterocycle may each be independently unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; and
with the provisos that R₆ and R₇ are not both H; and
when R is —SO₂R₈, R₇ is not H.

27. A compound having the formula III: 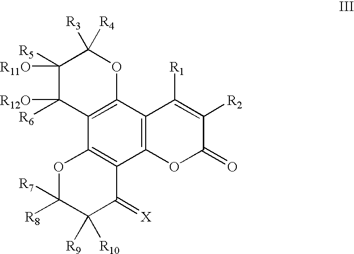
wherein R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen;
R₂ is H, halogen, hydroxyl, amino, thio, cyano, C₁₋₆ alkyl, aryl-C₁₋₆ alkyl, mono- or poly-fluorinated C₁₋₆ alkyl, aryl, or heterocycle; or
R₁ and R₂ together can form a 5-7 membered saturated or unsaturated cyclic ring or heterocyclic ring;
R₅ and R₆ are independently selected from the group consisting of H, C₁₋₆ alkyl, aryl-C₁₋₆ alkyl, mono- or poly-fluorinated C₁₋₆ alkyl, aryl, and heterocycle; or R₅ and R₆ together form a 5-7 membered saturated or unsaturated cyclic ring or heterocyclic ring;
R₃, R₄, R₇, R₈, R₉, and R₁₀ are independently selected from the groups consisting of H, halogen, hydroxyl, amino, nitro, thio, cyano, azido, C₁₋₆ alkyl, aryl-C₁₋₆ alkyl, mono- or poly-fluorinated C₁₋₆ alkyl, —C(O)R₁₃, —SO₂R₁₃, —R₁₃C(O)R₁₄, —R₁₃SO₂R₁₄, aryl, and heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; or
any of R₃ and R₄ together, R₇ and R₈ together, or R₉ and R₁₀ together can form a 5-7 membered saturated or unsaturated cyclic ring or heterocyclic ring;
R₁₁ and R₁₂ are H, C₁₋₆ alkyl, aryl-C₁₋₆ alkyl, mono- or poly-fluorinated C₁₋₆ alkyl, —C(O)R₁₃, —SO₂R₁₃, —P(O)(OR₁₃)₂, —R₁₃C(O)R₄, —R₁₃SO₂R₄, —R₁₃P(O)(OR₁₄)₂, amino acid, aryl, or heterocycle; wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; and
R₁₃ and R₁₄ are independently selected from the group consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, and heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; and
X is H, halogen, OH, O, SH, NH₂, NHOH, ═NOH, or NR₁₁R₁₂ wherein R₁₁ and R₁₂ are defined as above, or R₁₁ and R₁₂ together form a 5-7 membered saturated or unsaturated cyclic ring or heterocyclic ring.

28. A compound having the formula II: 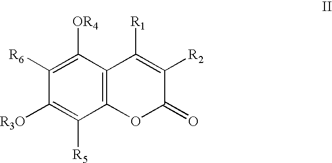
wherein R₁ is H, halogen, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, or heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen;
R₂ is H, halogen, hydroxyl, amino, thio, cyano, C₁₋₆ alkyl, aryl-C₁₋₆ alkyl, mono- or poly-fluorinated C₁₋₆ alkyl, aryl, or heterocycle; or R₁ and R₂ together can form a 5-7 membered saturated or unsaturated cyclic ring or heterocyclic ring;
R₃ and R₄ are independently selected from the group consisting of H, C₁₋₆ alkyl, aryl-C₁₋₆ alkyl, mono- or poly-fluorinated C₁₋₆ alkyl, aryl, heterocycle, —C(O)R₇, —SO₂R₇, —P(O)(OR₇)₂, —P(O)(OR₇)(OR₈), —R₇C(O)R₈, —R₇SO₂R₈, and —R₇P(O)(ORS)₂, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)-amino-C₁₋₈ alkyl, nitro, thio, cyano, azido, and halogen;
R₅ and R₆ are independently selected from the group consisting of H. halogen, hydroxyl, amino, nitro, thio, cyano, azido, C₁₋₆ alkyl, aryl-C₁₋₆ alkyl, mono- or poly-fluorinated C₁₋₆ alkyl, aryl, heterocycle, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl) amino-C₁₋₈ alkyl, —C(O)R₇, —SO₂R₇, —P(O)(OR₇)₂, —P(O)(OR₇)(OR₈), —R₇C(O)R₈, —R₇SO₂R₈, and —R₇P(O)(OR₈)₂—;
R₇ and R₈ are independently selected from the group consisting of H, hydroxyl, amino, thio, cyano, C₁₋₆ alkyl, aryl-C₁₋₆ alkyl, mono- or poly-fluorinated C₁₋₆ alkyl, hydroxy-C₁₋₆ alkyl, C₁₋₆ alkoxy, amino-C₁₋₈ alkyl, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclohexyl, aryl, and heterocycle, wherein aryl or heterocycle may each independently be unsubstituted or substituted with one or more from the group consisting of: C₁₋₆ alkyl, C₁₋₆ alkoxy, hydroxy-C₁₋₄ alkyl, hydroxyl, amino, C₁₋₆ alkylamino, di(C₁₋₆ alkyl)amino, amino-C₁₋₈ alkyl, C₁₋₈ alkylamino-C₁₋₈ alkyl, di(C₁₋₆ alkyl)amino-C₁₋₈ alkyl, nitro, thio, cyano, azido, and halogen; and
with a proviso that R₄, R₅ and R₆ are not each H.
